(19) United States
(12) Patent Application Publication
(10) Pub. No.: US 2020/0369770 A1
(43) Pub. Date: Nov. 26, 2020

(54) MULTISPECIFIC ANTIGEN BINDING PROTEINS AND METHODS OF USE THEREOF

(71) Applicant: Nanjing Legend Biotech Co., Ltd., Nanjing, Jiangsu (CN)

(72) Inventors: Yafeng ZHANG, Nanjing, Jiangsu (CN); Chuan-Chu CHOU, Westfield, NJ (US); Qi PAN, New York, NY (US); Shu WU, Nanjing, Jiangsu (CN); Shuai YANG, Nanjing, Jiangsu (CN)

(21) Appl. No.: 16/960,521

(22) PCT Filed: Jan. 8, 2019
(86) PCT No.: PCT/CN2019/070873

§ 371 Date: Jul. 7, 2020

(30) Foreign Application Priority Data
Jan. 8, 2018 (CN) PCT/CN2018/071729

(51) Int. Cl. C07K 16/28

(52) U.S. Cl. CPC C07K 16/2818; C07K 16/2809; C07K 16/2803; C07K 2317/31; C07K 2317/76; C07K 2317/52; C07K 2317/94; C07K 2317/92; C07K 2317/569

(57) ABSTRACT

Provided are multispecific, such as trispecific, antigen binding proteins comprising a first antigen binding domain comprising a heavy chain variable domain and a light chain variable domain, a second antigen binding domain comprising a first single-domain antibody, and a third antigen binding domain comprising a second single-domain antibody. Pharmaceutical compositions comprising the multispecific antigen binding proteins, kits and methods of use thereof are further provided.

### CROSS REFERENCE TO RELATED APPLICATIONS

This application claims priority benefits of International Patent Applications No. PCT/CN2018/071729 filed on Jan. 8, 2018, the contents of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761422001041SEQLIST.txt, date recorded: Jan. 4, 2019, size: 88 KB).

### FIELD OF THE INVENTION

The present invention relates to multispecific antigen binding proteins that specifically bind to three or more different antigens or epitopes and methods of use thereof.

### BACKGROUND OF THE INVENTION

Monoclonal antibodies (mAbs) have been widely used as therapeutic agents to treat a variety of human diseases, such as cancer and autoimmune diseases. Currently, there are more than 30 monoclonal antibodies including murine, fully humanized, and chimeric antibodies that have been approved by the FDA for therapeutic use. Rituximab and trastuzumab are among the top-selling protein therapeutics against cancer. Recently, monoclonal antibodies targeting immune checkpoint molecules, such as ipilimumab and nivolumab, have shown encouraging clinical results by inducing T cell immunity against tumors. As many patients do not respond well to monotherapy approaches, monoclonal antibodies are often combined with other immunomodulatory approaches, such as monoclonal antibodies against other targets, to enhance their efficacy. For example, clinical studies have demonstrated that combination of nivolumab and ipilimumab results in improved rates of objective response among melanoma patients.

Multispecific antibodies have been designed to simultaneously modulate two or more therapeutic targets in order to provide enhanced therapeutic efficacy and broadened potential utility. It has been reported that bispecific antibodies can be more effective than simple combination of two monoclonal antibodies. A variety of multispecific antibody formats have been developed. For example, bispecific antibodies have been made by fusing antigen binding (Fab) fragments or single chain variable fragments (scFvs) to monoclonal antibodies (see, for example, Weidle et al. Cancer Genomics & Proteomics 2013; 10: 1-18). Multispecific antibodies of different formats differ in size, are frequently produced by different technologies, and have different in vivo distribution, tissue penetration, and pharmacokinetic properties.

Despite their conceptual advantages, current multispecific antibodies are challenging to manufacture and develop as biologic drugs. As artificial constructs, multispecific antibodies cannot be produced by normal B-cells. Initial attempts to produce multispecific antibodies involved chemical conjugation of monospecific antibodies and fusion of mAb-expressing cells, but these approaches suffer from low efficiency and the necessity of purification from abundant side products. Advanced methods in protein engineering and molecular biology have enabled recombinant construction of a variety of new multispecific antibody formats. However, once adopted in these known engineered multispecific antibody formats, the individual components, such as scFvs and mAbs, lose their favorable biochemical and/or biophysical properties, serum half-life, and/or stability, resulting in poor efficacy, instability and high immunogenicity.

Single-domain antibodies (sdAbs) are antibody fragments each having a single monomeric antibody variable domain. Despite their much smaller sizes than common monoclonal antibodies having two heavy chains and two light chains, sdAbs can bind antigens with similar affinity and specificity as mAbs. Used as building blocks, the sdAbs can be fused to IgG Fc domains to create IgG-like antibodies, including bivalent and bispecific antibodies (see, for example, Hmila I. et al. Mol. Immunol. 2008; 45: 3847-3856).

The disclosures of all publications, patents, patent applications and published patent applications referred to herein are hereby incorporated herein by reference in their entirety.

### BRIEF SUMMARY OF THE INVENTION

The present application provides multispecific (such as trispecific) antigen binding proteins comprising two or more different single-domain antibodies (sdAbs) fused to a full-length four-chain antibody or an antigen binding fragment derived therefrom.

Accordingly, one aspect of the present application provides a multispecific (such as trispecific) antigen binding protein (“MABP”) comprising: (a) a first antigen binding portion comprising a heavy chain variable domain (V_{H}) and a light chain variable domain (V_{L}), wherein the V_{H} and V_{L} together form an antigen-binding site that specifically binds a first epitope, (b) a second antigen binding portion comprising a first single-domain antibody (sdAb) that specifically binds a second epitope, and (c) a third antigen binding portion comprising a second single-domain antibody (sdAb) that specifically binds a third epitope, wherein the first antigen binding portion, the second antigen binding portion, and the third antigen binding portion are fused to each other. In some embodiments, the first epitope, the second epitope and the third epitope are from the same antigen. In some embodiments, the first epitope, the second epitope and the third epitope are from different antigens. In some embodiments, the second epitope and the third epitope are from the same antigen, and the first epitope is from a different antigen. In some embodiments, the first epitope and the third epitope are from same antigen, the second epitope is from a different antigen.

In some embodiments according to any one of the MABPs described above, the first antigen binding portion is a full-length antibody consisting of two heavy chains and two light chains. In some embodiments, the first antigen binding portion is an antibody fragment comprising a heavy chain comprising the V_{H} and a light chain comprising the V_{L}. In some embodiments, the second antigen binding portion and/or the third antigen binding portion comprises a single polypeptide chain. In some embodiments, the first sdAb and/or the second sdAb is a V_{H}H.

In some embodiments according to any one of the MABPs described above, the MABP comprises: (1) a first polypeptide comprising from the N-terminus to the C-terminus: the first sdAb, and a heavy chain of the first antigen binding portion; and (2) a second polypeptide comprising from the N-terminus to the C-terminus: the second sdAb, and a light chain of the first antigen binding portion. In some embodiments, the MABP comprises: (1) a first polypeptide comprising from the N-terminus to the C-terminus: a heavy chain of the first antigen binding portion, and the first sdAb; and (2) a second polypeptide comprising from the N-terminus to the C-terminus: the second sdAb, and a light chain of the first antigen binding portion. In some embodiments, the MABP comprises: (1) a first polypeptide comprising from the N-terminus to the C-terminus: the first sdAb, and a heavy chain of the first antigen binding portion; and (2) a second polypeptide comprising from the N-terminus to the C-terminus: a light chain of the first antigen binding portion, and the second sdAb. In some embodiments, the MABP comprises: (1) a first polypeptide comprising from the N-terminus to the C-terminus: a heavy chain of the first antigen binding portion, and the first sdAb; and (2) a second polypeptide comprising from the N-terminus to the C-terminus: a light chain of the first antigen binding portion, and the second sdAb. In some embodiments, the MABP comprises: (1) a first polypeptide comprising from the N-terminus to the C-terminus: the first sdAb, a heavy chain of the first antigen binding portion, and the second sdAb; and (2) a second polypeptide comprising from the N-terminus to the C-terminus: a light chain of the first antigen binding portion. In some embodiments, the MABP comprises: (1) a first polypeptide comprising from the N-terminus to the C-terminus: a heavy chain of the first antigen binding portion; and (2) a second polypeptide comprising from the N-terminus to the C-terminus: the first sdAb, a light chain of the first antigen binding portion, and the second sdAb. In some embodiments, the MABP comprises: (1) a first polypeptide comprising from the N-terminus to the C-terminus: the first sdAb, the second sdAb, and a heavy chain of the first antigen binding portion; and (2) a second polypeptide comprising from the N-terminus to the C-terminus: a light chain of the first antigen binding portion. In some embodiments, the MABP comprises: (1) a first polypeptide comprising from the N-terminus to the C-terminus: a heavy chain of the first antigen binding portion, the second sdAb, and the first sdAb; and (2) a second polypeptide comprising from the N-terminus to the C-terminus: a light chain of the first antigen binding portion. In some embodiments, the MABP comprises: (1) a first polypeptide comprising from the N-terminus to the C-terminus: a heavy chain of the first antigen binding portion; and (2) a second polypeptide comprising from the N-terminus to the C-terminus: the first sdAb, the second sdAb, and a light chain of the first antigen binding portion. In some embodiments, the MABP comprises: (1) a first polypeptide comprising from the N-terminus to the C-terminus: a heavy chain of the first antigen binding portion; and (2) a second polypeptide comprising from the N-terminus to the C-terminus: a light chain of the first antigen binding portion, the second sdAb, and the first sdAb. In some embodiments, the MABP comprises two chains of the first polypeptide and two chains of the second polypeptide.

In some embodiments according to any one of the MABPs described above, the first epitope, the second epitope and/or the third epitope is from an immune checkpoint molecule. In some embodiments, the immune checkpoint molecule is selected from the group consisting of PD-1, PD-L1, PD-L2, CTLA-4, B7-H3, TIGIT, TIM-3, LAG-3, TIGIT, VISTA, ICOS, 4-1BB, OX40, GITR, and CD40. In some embodiments, the first antigen binding portion is an anti-PD-1 antibody or antigen binding fragment thereof. In some embodiments, the anti-PD-1 antibody is derived from pembrolizumab (e.g., KEYTRUDA®). In some embodiments, the second antigen binding portion comprises an anti-TIGIT sdAb. In some embodiments, the anti-TIGIT sdAb comprises the amino acid sequence of SEQ ID NO: 31, or a variant thereof comprising up to about 3 (such as about any of 1, 2, or 3) amino acid substitutions. In some embodiments, the anti-TIGIT sdAb comprises the amino acid sequence of SEQ ID NO: 31. In some embodiments, the third antigen binding portion comprises an anti-LAG-3 sdAb. In some embodiments, the anti-LAG-3 sdAb comprises the amino acid sequence of SEQ ID NO: 32, or a variant thereof comprising up to about 3 (such as about any of 1, 2, or 3) amino acid substitutions. In some embodiments, the anti-LAG-3 sdAb comprises the amino acid sequence of SEQ ID NO: 32. In some embodiments, the MABP comprises: (1) a first polypeptide comprising the amino acid sequence of any one of SEQ ID NOs: 16, 18, 20, 23, 25, 26, 28, or a variant thereof comprising up to about 5 (such as about any of 1, 2, 3, 4 or 5) amino acid substitutions; and (2) a second polypeptide comprising the amino acid sequence of any one of SEQ ID NOs: 15, 17, 19, 21, 22, 24, 27, or a variant thereof comprising up to about 5 (such as about any of 1, 2, 3, 4 or 5) amino acid substitutions. In some embodiments, the MABP comprises: (1) a first polypeptide comprising the amino acid sequence of any one of SEQ ID NOs: 16, 18, 20, 23, 25, 26 or 28; and (2) a second polypeptide comprising the amino acid sequence of any one of SEQ ID NOs: 15, 17, 19, 21, 22, 24 or 27.

In some embodiments according to any one of the MABPs described above, the first epitope, the second epitope and/or the third epitope is from a tumor antigen. In some embodiments, the tumor antigen is selected from the group consisting of HER2, BRAF, EGFR, VEGFR2, CD20, RANKL, CD38, and CD52. In some embodiments, the first epitope, the second epitope and/or the third epitope is from a cell surface antigen of an immune effector cell (such as T cell). In some embodiments, the first antigen binding portion is an anti-HER2 antibody or antigen binding fragment thereof. In some embodiments, the anti-HER2 antibody is derived from trastuzumab. In some embodiments, the second antigen binding portion comprises an anti-CD3 sdAb. In some embodiments, the third antigen binding portion comprises an anti-EGFR sdAb.

In some embodiments according to any one of the MABPs described above, the first epitope, the second epitope and/or the third epitope is from a pro-inflammatory molecule. In some embodiments, the pro-inflammatory molecule is selected from the group consisting of IL-113, TNF-α, IL-5, IL-6, IL-6R, IL-17A, IL-17F and eotaxin-1. In some embodiments, the first antigen binding portion is an anti-TNF-α antibody or antigen binding fragment thereof. In some embodiments, the anti-TNF-α antibody is derived from adalimumab. In some embodiments, the second antigen binding portion comprises an anti-IL-17A sdAb. In some embodiments, the third antigen binding portion comprises an anti-IL-17F sdAb.

In some embodiments according to any one of the MABPs described above, the first epitope, the second epitope and/or the third epitope is from an angiogenesis factor. In some embodiments, the first antigen binding portion is an anti-Ang2 antibody or antigen binding fragment thereof. In some embodiments, the anti-Ang2 antibody is derived from LC10. In some embodiments, the second antigen binding portion comprises an anti-VEGF sdAb. In some embodiments, the third antigen binding portion comprises an anti-DLL4 sdAb.

In some embodiments according to any one of the MABPs described above, the first antigen binding portion comprises a human, humanized or chimeric antibody or antigen binding fragment thereof.

In some embodiments according to any one of the MABPs described above, the first antigen binding portion comprises an Fc region. In some embodiments, the Fc region is an IgG1 Fc. In some embodiments, the Fc region is an IgG4 Fc, such as an IgG4 Fc having an S228P mutation.

In some embodiments according to any one of the MABPs described above, the first antigen binding portion, the second antigen binding portion, and/or the third antigen binding portion are fused to each other via a peptide bond or a peptide linker. In some embodiments, the peptide linker is no more than about 30 (such as no more than about any one of 25, 20 or 15) amino acids long. In some embodiments, the peptide linker comprises any one of the amino acid sequences of SEQ ID NOs: 1-7. In some embodiments, the first antigen binding portion, the second antigen binding portion and/or the third antigen binding portion are fused to each other chemically.

In some embodiments according to any one of the MABPs described above, the first sdA band/or the second sdAb is a camelid, humanized, or human sdAb.

In some embodiments according to any one of the MABPs described above, the MABP can be produced recombinantly, such as in mammalian cells (e.g., CHO cells), at an expression level of at least about 10 mg/L, such as at least about 10 mg/L, 50 mg/L, 100 mg/mL, or higher. In some embodiments, the MABP has an aggregation onset temperature (T_{agg}) of at least about 55° C., such as about 55° C. to about 70° C. In some embodiments, the MABP is stable for at least about one week at about 25° C. at a concentration of at least about 50 mg/mL. In some embodiments, the MABP is stable for at least about one week at 37° C. at a concentration of at least about 50 mg/mL. In some embodiments, the MABP is stable after at least about 5 freeze-thaw cycles at a concentration of at least 50 mg/mL. In some embodiments, the MABP has a high long-term stability in human serum for at least about any one of 1 day, 3 days or 7 days at physiological temperature, e.g., about 37° C.

Another aspect of the present application provides a pharmaceutical composition comprising any one of the MABPs described above and a pharmaceutically acceptable carrier. In some embodiments, the concentration of the MABP in the pharmaceutical composition is at least about 100 mg/mL, such as at least about 150 mg/mL, 200 mg/mL or higher.

Further provided in one aspect of the present application is a method of treating a disease in an individual, comprising administering to the individual an effective amount of any one of the pharmaceutical compositions described above. In some embodiments, the disease is a cancer. In some embodiments, the cancer is selected from the group consisting of breast cancer, renal cancer, melanoma, lung cancer, glioblastoma, head and neck cancer, prostate cancer, ovarian carcinoma, bladder carcinoma, and lymphoma. In some embodiments, the disease is an inflammatory or autoimmune disease. In some embodiments, the inflammatory or autoimmune disease is selected from the group consisting of arthritis (such as rheumatoid arthritis, juvenile idiopathic arthritis, psoriatic arthritis, ankylosing spondylitis, and arthritic ulcerative colitis), colitis, psoriasis, severe asthma, and moderate to severe Crohn's disease.

### BRIEF DESCRIPTION OF THE DRAWINGS

FIG. 1 depicts a schematic structure of an exemplary trispecific antigen binding protein (also referred herein as “TABP”) comprising a monospecific full-length antibody having two identical heavy chains and two identical light chains, a first sdAb and a second sdAb, wherein the C-terminus of the first sdAb is fused to the N-terminus of the heavy chain via a first optional peptide linker and the C-terminus of the second sdAb is fused to the N-terminus of the light chain via a second optional peptide linker. The full-length antibody has two antigen binding sites that specifically bind the first epitope. The first sdAb specifically binds the second epitope. The second sdAb specially binds to the third epitope. For example, the TABP can consist of four polypeptide chains with structures from the N-terminus to the C-terminus as follows: (1) V_{H}H2-V_{L}-C_{L}; (2) V_{H}H1-V_{H}-C_{H}1-C_{H}2-C_{H}3; (3) V_{H}H1-V_{H}-C_{H}1-C_{H}2-C_{H}3; and (4) V_{H}H2-V_{L}-C_{L}.

FIG. 2 depicts a schematic structure of an exemplary trispecific antigen binding protein comprising a monospecific full-length antibody having two identical heavy chains and two identical light chains, a first sdAb, and a second sdAb, wherein the C-terminus of the first sdAb is fused to the N-terminus of the second sdAb via a first optional peptide linker, and the C-terminus of the second sdAb is fused to the N-terminus of the heavy chain via a second optional peptide linker. The full-length antibody has two antigen binding sites that specifically bind the first epitope. The first sdAb specifically binds the second epitope. The second sdAb specially binds to the third epitope. For example, the TABP can consist of four polypeptide chains with structures from the N-terminus to the C-terminus as follows: (1) V_{L}-C_{L}; (2) V_{H}H1-V_{H}H2-V_{H}-C_{H}1-C_{H}2-C_{H}3; (3) V_{H}H1-V_{H}H2-V_{H}-C_{H}1-C_{H}2-C_{H}3; and (4) V_{L}-C_{L}.

FIG. 3 depicts a schematic structure of an exemplary trispecific antigen binding protein comprising a monospecific full-length antibody having two identical heavy chains and two identical light chains, a first sdAb, and a second sdAb, wherein the C-terminus of the first sdAb is fused to the N-terminus of the second sdAb via a first optional peptide linker, and the C-terminus of the second sdAb is fused to the N-terminus of the light chain via a second optional peptide linker. The full-length antibody has two antigen binding sites that specifically bind the first epitope. The first sdAb specifically binds the second epitope. The second sdAb specially binds to the third epitope. For example, the TABP can consist of four polypeptide chains with structures from the N-terminus to the C-terminus as follows: (1) V_{H}H1-V_{H}H2-V_{L}-C_{L}; (2) V_{H}-C_{H}1-C_{H}2-C_{H}3; (3) V_{H}-C_{H}1-C_{H}2-C_{H}3; and (4) V_{H}H1-V_{H}H2-V_{L}-C_{L}.

FIG. 4 depicts a schematic structure of an exemplary trispecific antigen binding protein comprising a monospecific full-length antibody having two identical heavy chains and two identical light chains, a first sdAb and a second sdAb, wherein the C-terminus of the first sdAb is fused to the N-terminus of the light chain via a first optional peptide linker, and the N-terminus of the second sdAb is fused to the C-terminus of the light chain via a second optional peptide linker. The full-length antibody has two antigen binding sites that specifically bind the first epitope. The first sdAb specifically binds the second epitope. The second sdAb specially binds to the third epitope. For example, the TABP can consist of four polypeptide chains with structures from the N-terminus to the C-terminus as follows: (1) V_{H}H1-V_{L}-C_{L}-V_{H}H2; (2) V_{H}-C_{H}1-C_{H}2-C_{H}3; (3) V_{H}-C_{H}1-C_{H}2-C_{H}3; and (4) V_{H}H1-V_{L}-C_{L}-V_{H}H2.

FIG. 5 depicts a schematic structure of an exemplary trispecific antigen binding protein comprising a monospecific full-length antibody having two identical heavy chains and two identical light chains, a first sdAb and a second sdAb, wherein the C-terminus of the first sdAb is fused to the N-terminus of the heavy chain via a first optional peptide linker and the N-terminus of the second sdAb is fused to the C-terminus of the light chain via a second optional peptide linker. The full-length antibody has two antigen binding sites that specifically bind the first epitope. The first sdAb specifically binds the second epitope. The second sdAb specially binds to the third epitope. For example, the TABP can consist of four polypeptide chains with structures from the N-terminus to the C-terminus as follows: (1) V_{L}-C_{L}-V_{H}H2; (2) V_{H}H1-V_{H}-C_{H}1-C_{H}2-C_{H}3; (3) V_{H}H1-V_{H}-C_{H}1-C_{H}2-C_{H}3; and (4) V_{L}-C_{L}-V_{H}H2.

FIG. 6 depicts a schematic structure of an exemplary trispecific antigen binding protein comprising a monospecific full-length antibody having two identical heavy chains and two identical light chains, a first sdAb and a second sdAb, wherein the C-terminus of the first sdAb is fused to the N-terminus of the heavy chain via a first optional peptide linker and the N-terminus of the second sdAb is fused to the C-terminus of the heavy chain via a second optional peptide linker. The full-length antibody has two antigen binding sites that specifically bind the first epitope. The first sdAb specifically binds the second epitope. The second sdAb specially binds to the third epitope. For example, the TABP can consist of four polypeptide chains with structures from the N-terminus to the C-terminus as follows: (1) V_{L}-C_{L}; (2) V_{H}H1-V_{H}-C_{H}1-C_{H}2-C_{H}3-V_{H}H2; (3) V_{H}H1-V_{H}-C_{H}1-C_{H}2-C_{H}3-V_{H}H2; and (4) V_{L}-C_{L}.

FIG. 7 depicts a schematic structure of an exemplary trispecific antigen binding protein comprising a monospecific full-length antibody having two identical heavy chains and two identical light chains, a first sdAb and a second sdAb, wherein the C-terminus of the first sdAb is fused to the N-terminus of the light chain via a first optional peptide linker and the N-terminus of the second sdAb is fused to the C-terminus of the heavy chain via a second optional peptide linker. The full-length antibody has two antigen binding sites that specifically bind the first epitope. The first sdAb specifically binds the second epitope. The second sdAb specially binds to the third epitope. For example, the TABP can consist of four polypeptide chains with structures from the N-terminus to the C-terminus as follows: (1) V_{H}H1-V_{L}-C_{L}; (2) V_{H}-C_{H}1-C_{H}2-C_{H}3-V_{H}H2; (3) V_{H}-C_{H}1-C_{H}2-C_{H}3-V_{H}H2; and (4) V_{H}H1-V_{L}-C_{L}.

FIG. 8 depicts a schematic structure of an exemplary trispecific antigen binding protein comprising a monospecific full-length antibody having two identical heavy chains and two identical light chains, a first sdAb and a second sdAb, wherein the N-terminus of the first sdAb is fused to the C-terminus of the heavy chain via a first optional peptide linker and the N-terminus of the second sdAb is fused to the C-terminus of the light chain via a second optional peptide linker. The full-length antibody has two antigen binding sites that specifically bind the first epitope. The first sdAb specifically binds the second epitope. The second sdAb specially binds to the third epitope. For example, the TABP can consist of four polypeptide chains with structures from the N-terminus to the C-terminus as follows: (1) V_{L}-C_{L}-V_{H}H2; (2) V_{H}-C_{H}1-C_{H}2-C_{H}3-V_{H}H1; (3) V_{H}-C_{H}1-C_{H}2-C_{H}3-V_{H}H1; and (4) V_{L}-C_{L}-V_{H}H2.

FIG. 9 depicts a schematic structure of an exemplary trispecific antigen binding protein comprising a monospecific full-length antibody having two identical heavy chains and two identical light chains, a first sdAb and a second sdAb, wherein the N-terminus of the first sdAb is fused to the C-terminus of the second sdAb via a first optional peptide linker, and the N-terminus of the second sdAb is fused to the C-terminus of the light chain via a second optional peptide linker. The full-length antibody has two antigen binding sites that specifically bind the first epitope. The first sdAb specifically binds the second epitope. The second sdAb specially binds to the third epitope. For example, the TABP can consist of four polypeptide chains with structures from the N-terminus to the C-terminus as follows: (1) V_{L}-C_{L}-V_{H}H2-V_{H}H1; (2) V_{H}-C_{H}1-C_{H}2-C_{H}3; (3) V_{H}-C_{H}1-C_{H}2-C_{H}3; and (4) V_{L}-C_{L}-V_{H}H2-V_{H}H1.

FIG. 10 depicts a schematic structure of an exemplary trispecific antigen binding protein comprising a monospecific full-length antibody having two identical heavy chains and two identical light chains, a first sdAb and a second sdAb, wherein the N-terminus of the first sdAb is fused to the C-terminus of the second sdAb via a first optional peptide linker, and the N-terminus of the second sdAb is fused to the C-terminus of the heavy chain via a second optional peptide linker. The full-length antibody has two antigen binding sites that specifically bind the first epitope. The first sdAb specifically binds the second epitope. The second sdAb specially binds to the third epitope. For example, the TABP can consist of four polypeptide chains with structures from the N-terminus to the C-terminus as follows: (1) V_{L}-C_{L}; (2) V_{H}-C_{H}1-C_{H}2-C_{H}3-V_{H}H2-V_{H}H1; (3) V_{H}-C_{H}1-C_{H}2-C_{H}3-V_{H}H2-V_{H}H1; and (4) V_{L}-C_{L}.

FIG. 11 shows a table summarizing antibody production data of 10 exemplary TABPs. TPTL11 has the format shown in FIG. 1. TPTL12 has the format shown in FIG. 2. TPTL13 has the format shown in FIG. 3. TPTL14 has the format shown in FIG. 4. TPTL15 has the format shown in FIG. 5. TPTL16 has the format shown in FIG. 6. TPTL17 has the format shown in FIG. 7. TPTL18 has the format shown in FIG. 8. TPTL19 has the format shown in FIG. 9. TPTL20 has the format shown in FIG. 10.

FIGS. 12A-12K show binding curves of exemplary TABPs (TPTL11-TPTL20) and KEYTRUDA® (positive control) respectively to PD-1 as measured by BIACORE® T200.

FIGS. 13A-13K show binding curves of exemplary TABPs (TPTL11-TPTL20) and AS19584VH28 HCAb (positive control) respectively to TIGIT as measured by BIACORE® T200.

FIGS. 14A-14K show binding curves of exemplary TABPs (TPTL11-TPTL20) and VHH2 HCAb (positive control) respectively to LAG-3 as measured by BIACORE® T200.

FIG. 15 shows in vitro binding and ligand competition parameters of exemplary TABPs (TPTL11-TPTL20) to PD-1, TIGIT, and LAG-3.

FIG. 16 shows thermal stability of exemplary TABPs (TPTL11-TPTL20) by temperature-induced aggregation.

FIG. 17 shows stability of exemplary TABPs (TPTL11-TPTL20) after 5 Freeze-Thaw cycles.

FIGS. 18A-18C show human serum stability of exemplary TABPs (TPTL11-TPTL17) after serum co-incubation 1 day, 7 days and 14 days. FIG. 18A shows the binding activity of exemplary TABPs (TPTL11-TPTL17) to human TIGIT protein. FIG. 18B shows the binding activity of exemplary TABPs (TPTL11-TPTL17) to human LAG-3 protein. FIG. 18C shows the binding activity of exemplary TABPs (TPTL11-TPTL17) to human PD-1 protein.

### DETAILED DESCRIPTION OF THE INVENTION

The present application provides novel multispecific antigen binding proteins (“MABPs”) comprising two or more single-domain antibodies (sdAbs) fused to a full-length antibody or antigen binding fragment that comprises a heavy chain variable domain (V_{H}) and a light chain variable domain (V_{L}). Each sdAb specifically binds a different target (such as different epitope or antigen), and the targets of the sdAbs are also distinct from the target recognized by the full-length antibody or antigen binding fragment. The multispecific antigen binding protein formats described herein enable multivalent co-engagement of distinct target antigens, and also provide novel homodimerization variants that facilitate folding and purification of homodimeric proteins such as antibodies.

Some embodiments of the present application provides trispecific antigen binding proteins (“TABPs”), which can be adopted to target a variety of disease-related epitope and/or antigen combinations, including a combination of immune checkpoint molecules, cell surface antigens (such as tumor antigens), angiogenic factors, or pro-inflammatory molecules. The TABPs described herein provide useful agents for treating a variety of diseases and conditions, such as cancer, inflammation, and autoimmune diseases.

Accordingly, one aspect of the present application provides a multispecific (e.g., trispecific) antigen binding protein comprising: (a) a first antigen binding portion comprising a heavy chain variable domain (V_{H}) and a light chain variable domain (V_{L}), wherein the V_{H} and V_{L} together form an antigen-binding site that specifically binds a first epitope, (b) a second antigen binding portion comprising a first single-domain antibody that specifically binds a second epitope, and (c) a third antigen binding portion comprising a second single-domain antibody that specifically binds a third epitope, wherein the first antigen binding portion, the second antigen binding portion and the third antigen binding portion are fused to each other.

In some embodiments, there is provided a multispecific (e.g., trispecific) antigen binding protein comprising: (a) a first antigen binding portion comprising a heavy chain variable domain (V_{H}) and a light chain variable domain (V_{L}), wherein the V_{H} and V_{L} together form an antigen-binding site that specifically binds a first immune checkpoint molecule (e.g., PD-1), (b) a second antigen binding portion comprising a first single-domain antibody that specifically binds a second immune checkpoint molecule (e.g., TIGIT), and (c) a third antigen binding portion comprising a second single-domain antibody that specifically binds a third immune checkpoint molecule (e.g., LAG-3), wherein the first antigen binding portion, the second antigen binding portion and the third antigen binding portion are fused to each other.

In some embodiments, there is provided a multispecific (e.g., trispecific) antigen binding protein (TABP) comprising: (a) a first antigen binding portion comprising a heavy chain variable domain (V_{H}) and a light chain variable domain (V_{L}), wherein the V_{H} and V_{L} together form an antigen-binding site that specifically binds a first tumor antigen (e.g., HER-2), (b) a second antigen binding portion comprising a first single-domain antibody that specifically binds a second tumor antigen or a cell surface antigen of an immune effector cell (e.g., CD-3), and (c) a third antigen binding portion comprising a second single-domain antibody that specifically binds a third tumor antigen (e.g., EGFR), wherein the first antigen binding portion, the second antigen binding portion and the third antigen binding portion are fused to each other.

In some embodiments, there is provided a multispecific (e.g., trispecific) antigen binding protein (TABP) comprising: (a) a first antigen binding portion comprising a heavy chain variable domain (V_{H}) and a light chain variable domain (V_{L}), wherein the V_{H} and V_{L} together form an antigen-binding site that specifically binds a first pro-inflammatory molecule (e.g., TNF-α), (b) a second antigen binding portion comprising a first single-domain antibody that specifically binds a second pro-inflammatory molecule (e.g., IL-17A), and (c) a third antigen binding portion comprising a second single-domain antibody that specifically binds a third pro-inflammatory molecule (e.g., IL-17F), wherein the first antigen binding portion, the second antigen binding portion and the third antigen binding portion are fused to each other.

In some embodiments, there is provided a multispecific (e.g., trispecific) antigen binding protein (TABP) comprising: (a) a first antigen binding portion comprising a heavy chain variable domain (V_{H}) and a light chain variable domain (V_{L}), wherein the V_{H} and V_{L} together form an antigen-binding site that specifically binds a first angiogenic factor (e.g., Ang2), (b) a second antigen binding portion comprising a first single-domain antibody that specifically binds a second angiogenic factor (e.g., VEGF), and (c) a third antigen binding portion comprising a second single-domain antibody that specifically binds a third angiogenic factor (e.g., DLL4), wherein the first antigen binding portion, the second antigen binding portion and the third antigen binding portion are fused to each other.

Also provided are pharmaceutical compositions, kits and articles of manufacture comprising the multispecific (e.g., trispecific) antigen binding proteins, and methods of treating a disease using the multispecific (e.g., trispecific) antigen binding proteins described herein.

### I. Definitions

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Current Protocols in Molecular Biology or Current Protocols in Immunology, John Wiley & Sons, New York, N.Y. (2009); Ausubel et al, Short Protocols in Molecular Biology, 3^{rd} ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

The terms “multispecific antigen binding protein” and “MABP” are used interchangeably herein. The terms “trispecific antigen binding protein” and “TABP” are used interchangeably herein.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” by the MABP of the present application if one or more symptoms associated with the disease or condition being treated (such as cancer, inflammatory or autoimmune disease) are mitigated or eliminated.

As used herein, an “effective amount” refers to an amount of an agent or drug effective to treat a disease or condition in a subject. In the case of cancer, the effective amount of the MABP of the present application may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, an “individual” or a “subject” refers to a mammal, including, but not limited to, human, bovine, horse, feline, canine, rodent, or primate. In some embodiments, the individual is a human.

The term “antibody” includes monoclonal antibodies (including full length 4-chain antibodies which have an immunoglobulin Fc region), antibody compositions with polyepitopic specificity, multispecific antibodies (e.g., bispecific or trispecific antibodies), diabodies, and single-chain molecules, as well as antibody fragments (e.g., Fab, F(ab′)₂, and Fv). The term “immunoglobulin” (Ig) is used interchangeably with “antibody” herein. Antibodies contemplated herein include heavy-chain only antibodies and single-domain antibodies.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Ten and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1 and IgA2.

An “isolated” antibody is one that has been identified, separated and/or recovered from a component of its production environment (e.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or nonproteinaceous solutes. In preferred embodiments, the polypeptide will be purified: (1) to greater than 95% by weight of antibody as determined by, for example, the Lowry method, and in some embodiments, to greater than 99% by weight; (1) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant cells since at least one component of the antibody's natural environment will not be present. Ordinarily, however, an isolated polypeptide or antibody will be prepared by at least one purification step.

The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites. Heavy-chain only antibodies from the Camelidae species have a single heavy chain variable region, which is referred to as “V_{H}H”. V_{H}H is thus a special type of V_{H}.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present application may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The terms “full-length antibody,” “intact antibody” or “whole antibody” are used interchangeably to refer to an antibody in its substantially intact form, as opposed to an antibody fragment. Specifically full-length 4-chain antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of the antibodies described herein comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains Diabodies are described in greater detail in, for example, EP 404,097; WO 93/11161; Hollinger et al., Proc. Natl. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATTZFD® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, 4-chain antibodies comprise six HVRs; three in the V_{H} (H1, H2, H3), and three in the V_{L} (L1, L2, L3). Single-domain antibodies comprise three HVRs, such as three in the V_{H}H (H1, H2, H3). In native 4-chain antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

The term “Complementarity Determining Region” or “CDR” are used to refer to hypervariable regions as defined by the Kabat system. See Kabat et al., Sequences of Proteins of Immunological Interest, 5th Ed. Public Health Service, National Institutes of Health, Bethesda, Md. (1991)

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below in Table 1.

TABLE 1
HVR delineations.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |

(Kabat Numbering)

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the V_{L} and 26-35 (H1), 50-65 or 49-65 (H2) and 93-102, 94-102, or 95-102 (H3) in the V_{H}. The variable domain residues are numbered according to Kabat et al., supra, for each of these definitions.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

“Framework” or “FR” residues are those variable-domain residues other than the HVR residues as herein defined.

A “human consensus framework” or “acceptor human framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5^{th} Ed. Public Health Service, National Institutes of Health, Bethesda, Md. (1991). Examples include for the V_{L}, the subgroup may be subgroup kappa I, kappa II, kappa III or kappa IV as in Kabat et al., supra. Additionally, for the VH, the subgroup may be subgroup I, subgroup II, or subgroup III as in Kabat et al. Alternatively, a human consensus framework can be derived from the above in which particular residues, such as when a human framework residue is selected based on its homology to the donor framework by aligning the donor framework sequence with a collection of various human framework sequences. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less.

An “amino-acid modification” at a specified position, e.g. of the Fc region, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by V_{H}- and V_{L}-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As use herein, the term “specifically binds” or is “specific for” refers to measurable and reproducible interactions such as binding between a target and an antibody, which is determinative of the presence of the target in the presence of a heterogeneous population of molecules including biological molecules. For example, an antibody that specifically binds a target (which can be an epitope) is an antibody that binds this target with greater affinity, avidity, more readily, and/or with greater duration than it binds other targets. In one embodiment, the extent of binding of an antibody to an unrelated target is less than about 10% of the binding of the antibody to the target as measured, e.g., by a radioimmunoassay (RIA). In certain embodiments, an antibody that specifically binds a target has a dissociation constant (Kₐ) of ≤1 μM, ≤100 nM, ≤10 nM, ≤1 nM, or ≤0.1 nM. In certain embodiments, an antibody specifically binds an epitope on a protein that is conserved among the protein from different species. In another embodiment, specific binding can include, but does not require exclusive binding.

The term “specificity” refers to selective recognition of an antigen binding protein or antibody for a particular epitope of an antigen. Natural antibodies, for example, are monospecific. The term “multispecific” as used herein denotes that an antigen binding protein or an antibody has two or more antigen-binding sites of which at least two bind a different antigen or a different epitope of the same antigen. “Trispecific” as used herein denotes that an antigen binding protein or an antibody has three different antigen-binding specificities. The term “monospecific” antibody as used herein denotes an antibody that has one or more binding sites each of which bind the same epitope of the same antigen.

The term “valent” as used herein denotes the presence of a specified number of binding sites in an antigen binding protein or antibody molecule. A natural antibody for example or a full length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein or antibody molecule.

A “blocking” antibody or an “antagonist” antibody is one that inhibits or reduces a biological activity of the antigen it binds. In some embodiments, blocking antibodies or antagonist antibodies substantially or completely inhibit the biological activity of the antigen.

An “agonist” or activating antibody is one that enhances or initiates signaling by the antigen to which it binds. In some embodiments, agonist antibodies cause or activate signaling without the presence of the natural ligand.

“Antibody effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptors); and B cell activation. “Reduced or minimized” antibody effector function means that which is reduced by at least 50% (alternatively 60%, 65%, 70%, 75%, 80%, 85%, 90%, 95%, 96%, 97%, 98%, 99%) from the wild type or unmodified antibody. The determination of antibody effector function is readily determinable and measurable by one of ordinary skill in the art. In a preferred embodiment, the antibody effector functions of complement binding, complement dependent cytotoxicity and antibody dependent cytotoxicity are affected. In some embodiments, effector function is eliminated through a mutation in the constant region that eliminated glycosylation, e.g., “effector-less mutation.” In one aspect, the effector-less mutation is an N297A or DANA mutation (D265A+N297A) in the C_{H}2 region. Shields et al., J. Biol. Chem. 276 (9): 6591-6604 (2001). Alternatively, additional mutations resulting in reduced or eliminated effector function include: K322A and L234A/L235A (LALA). Alternatively, effector function can be reduced or eliminated through production techniques, such as expression in host cells that do not glycosylate (e.g., E. coli.) or in which result in an altered glycosylation pattern that is ineffective or less effective at promoting effector function (e.g., Shinkawa et al., J. Biol. Chem. 278(5): 3466-3473 (2003).

“Antibody-dependent cell-mediated cytotoxicity” or ADCC refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ADCC, NK cells, express FcγRIII only, whereas monocytes express FcγR1, FcγRII and FcγRIII. Fc expression on hematopoietic cells is summarized in Table 3 on page 464 of Ravetch and Kinet, Annu. Rev. Immunol. 9: 457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al., PNAS USA 95:652-656 (1998).

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

“Fc receptor” or “FcR” describes a receptor that binds the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see M. Daeron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus. Guyer et al., J. Immunol. 117: 587 (1976) and Kim et al., J. Immunol. 24: 249 (1994). Methods of measuring binding to FcRn are known (see, e.g., Ghetie and Ward, Immunol. Today 18: (12): 592-8 (1997); Ghetie et al., Nature Biotechnology 15 (7): 637-40 (1997); Hinton et al., J. Biol. Chem. 279 (8): 6213-6 (2004); WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e.g., Shields et al., J. Biol. Chem. 9(2): 6591-6604 (2001).

“Effector cells” are leukocytes which express one or more FcRs and perform effector functions. In one aspect, the effector cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils. The effector cells may be isolated from a native source, e.g., blood. Effector cells generally are lymphocytes associated with the effector phase, and function to produce cytokines (helper T cells), killing cells in infected with pathogens (cytotoxic T cells) or secreting antibodies (differentiated B cells).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202: 163 (1996), may be performed. Antibody variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

The term “heavy chain-only antibody” or “HCAb” refers to a functional antibody, which comprises heavy chains, but lacks the light chains usually found in antibodies. Camelid animals (such as camels, llamas, or alpacas) are known to produce HCAbs.

The term “single-domain antibody” or “sdAb” refers to a single antigen-binding polypeptide having three complementary determining regions (CDRs). The sdAb alone is capable of binding to the antigen without pairing with a corresponding CDR-containing polypeptide. In some cases, sdAbs are engineered from camelid HCAbs, and their heavy chain variable domains are referred herein as “V_{H}Hs”. Camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)).

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). The affinity of a molecule X for its partner Y can generally be represented by the dissociation constant (K_{d}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present application. Specific illustrative and exemplary embodiments for measuring binding affinity are described in the following.

The “K_{d}” or “K_{d} value” as used herein is in one embodiment measured by a radiolabeled antigen binding assay (RIA) performed with the Fab version of the antibody and antigen molecule as described by the following assay that measures solution binding affinity of Fabs for antigen by equilibrating Fab with a minimal concentration of (¹²⁵I)-labeled antigen in the presence of a titration series of unlabeled antigen, then capturing bound antigen with an anti-Fab antibody-coated plate (Chen, et al., (1999) J. Mol. Biol 293:865-881). To establish conditions for the assay, microtiter plates (Dynex) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (consistent with assessment of an anti-VEGF antibody, Fab-12, in Presta et al., (1997) Cancer Res. 57:4593-4599). The Fab of interest is then incubated overnight; however, the incubation may continue for a longer period (e.g., 65 hours) to insure that equilibrium is reached. Thereafter, the mixtures are transferred to the capture plate for incubation at room temperature for one hour. The solution is then removed and the plate washed eight times with 0.1% Tween-20 in PBS. When the plates have dried, 150 of scintillant (MicroScint-20; Packard) is added, and the plates are counted on a Topcount gamma counter (Packard) for ten minutes. Concentrations of each Fab that give less than or equal to 20% of maximal binding are chosen for use in competitive binding assays.

According to another embodiment, the K_{d} is measured by using surface-plasmon resonance assays using a BIACORE®-T200 or a BIACORE®-3000 instrument (BIAcore, Inc., Piscataway, N.J.) at 25° C. with immobilized antigen CMS chips at ^{˜}10 response units (RU). Briefly, carboxymethylated dextran biosensor chips (CMS, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (^{˜}0.2 μM) before injection at a flow rate of 5 μL/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% TWEEN 20™ surfactant (PBST) at 25° C. at a flow rate of approximately 25 μL/min. Association rates (kₒₙ) and dissociation rates (k_{off}) are calculated using a simple one-to-one Langmuir binding model (BIAcore® Evaluation Software version 3.2) by simultaneously fitting the association and dissociation sensorgrams. The equilibrium dissociation constant (Kₐ) is calculated as the ratio k_{off}/kₒₙ. See, e.g., Chen et al., J. Mol. Biol. 293:865-881 (1999). If the on-rate exceeds 10⁶M⁻¹ s⁻¹ by the surface-plasmon resonance assay above, then the on-rate can be determined by using a fluorescent quenching technique that measures the increase or decrease in fluorescence-emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow-equipped spectrophotometer (Aviv Instruments) or a 8000-series SLM-AMINCO™ spectrophotometer (ThermoSpectronic) with a stirred cuvette.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An “isolated” nucleic acid molecule encoding the MABP herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or nonionic surfactants such as TWEEN™, polyethylene glycol (PEG), and PLURONICS™ or polyethylene glycol (PEG).

The “diluent” of interest herein is one which is pharmaceutically acceptable (safe and nontoxic for administration to a human) and is useful for the preparation of a liquid formulation, such as a formulation reconstituted after lyophilization. Exemplary diluents include sterile water, bacteriostatic water for injection (BWFI), a pH buffered solution (e.g. phosphate-buffered saline), sterile saline solution, Ringer's solution or dextrose solution. In an alternative embodiment, diluents can include aqueous solutions of salts and/or buffers.

A “preservative” is a compound which can be added to the formulations herein to reduce bacterial activity. The addition of a preservative may, for example, facilitate the production of a multi-use (multiple-dose) formulation. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The most preferred preservative herein is benzyl alcohol.

The term “pharmaceutical formulation” refers to a preparation that is in such form as to permit the biological activity of the active ingredient to be effective, and that contains no additional components that are unacceptably toxic to a subject to which the formulation would be administered. Such formulations are sterile. A “sterile” formulation is aseptic or free from all living microorganisms and their spores.

A “stable” formulation is one in which the protein therein essentially retains its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation may be kept at 40° C. for 2 weeks to 1 month, at which time stability is measured. Where the formulation is to be stored at 2-8° C., generally the formulation should be stable at 30° C. or 40° C. for at least 1 month and/or stable at 2-8° C. for at least 2 years. Where the formulation is to be stored at 30° C., generally the formulation should be stable for at least 2 years at 30° C. and/or stable at 40° C. for at least 6 months. For example, the extent of aggregation during storage can be used as an indicator of protein stability. Thus, a “stable” formulation may be one wherein less than about 10% and preferably less than about 5% of the protein are present as an aggregate in the formulation. In other embodiments, any increase in aggregate formation during storage of the formulation can be determined.

A “reconstituted” formulation is one which has been prepared by dissolving a lyophilized protein or antibody formulation in a diluent such that the protein is dispersed throughout. The reconstituted formulation is suitable for administration (e.g. subcutaneous administration) to a patient to be treated with the protein of interest and, in certain embodiments, may be one which is suitable for parenteral or intravenous administration.

An “isotonic” formulation is one which has essentially the same osmotic pressure as human blood. Isotonic formulations will generally have an osmotic pressure from about 250 to 350 mOsm. The term “hypotonic” describes a formulation with an osmotic pressure below that of human blood. Correspondingly, the term “hypertonic” is used to describe a formulation with an osmotic pressure above that of human blood. Isotonicity can be measured using a vapor pressure or ice-freezing type osmometer, for example. The formulations of the present application can be hypertonic as a result of the addition of salt and/or buffer.

“Immune checkpoint molecules” refers to molecules in the immune system that either turn up a signal or turn down a signal. “Stimulatory immune checkpoint molecules” or “co-stimulatory molecules” are immune checkpoint molecules that turn up a signal in the immune system. “Inhibitory immune checkpoint molecules” are immune checkpoint molecules that turn down a signal in the immune system.

It is understood that embodiments described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

### II. Multispecific Antigen Binding Proteins (MABPs)

One aspect of the present application provides a multispecific (e.g., trispecific) antigen binding protein (MABP) comprising: (a) a first antigen binding portion comprising a heavy chain variable domain (V_{H}) and a light chain variable domain (V_{L}), wherein the V_{H} and V_{L} together form an antigen-binding site that specifically binds a first epitope, (b) a second antigen binding portion comprising a first sdAb that specifically binds a second epitope, and (c) a third antigen binding portion comprising a second sdAb that specifically binds a third epitope, wherein the first antigen binding portion, the second antigen binding portion, and the third antigen binding portion are fused to each other. In some embodiments, the first epitope is from a first immune checkpoint molecule (e.g., PD-1, SEQ ID NO:12), the second epitope is from a second immune checkpoint molecule (e.g., TIGIT, SEQ ID NO:13), and the third epitope is from a third immune checkpoint molecule (e.g., LAG-3, SEQ ID NO:14). In some embodiments, the first epitope is from a first tumor antigen, the second epitope is from a second tumor antigen, and the third epitope is from a third tumor antigen. In some embodiments, the first epitope is from a first tumor antigen (e.g., HER-2), the second epitope is from a cell surface molecule on an immune effector cell (e.g., CD3), and the third epitope is from a second tumor antigen (e.g., EGFR). In some embodiments, the first epitope is from a first pro-inflammatory molecule (e.g., TNF-α), the second epitope is from a second pro-inflammatory molecule (e.g., IL-17A), and the third epitope is from a third pro-inflammatory molecule (e.g., IL-17F). In some embodiments, the first epitope is from a first angiogenic factor (e.g., Ang2), the second epitope is from a second angiogenic factor (e.g., VEGF), and the third epitope is from a third angiogenic factor (e.g., DLL4). In some embodiments, the first sdAb and/or the second sdAb is a V_{H}H. In some embodiments, the first antigen binding portion comprises a heavy chain comprising the V_{H} and a light chain comprising the V_{L}. In some embodiments, the first antigen binding region is a full-length antibody consisting of two heavy chains and two light chains. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG1 Fc or IgG4 Fc.

The MABPs of the present application have at least three antigen binding portions that can specifically bind at least three different epitopes. The MABPs can be symmetric or asymmetric. For example, the MABP may comprise one or two copies of the first antigen binding portion, one to eight copies of the second antigen binding portion, and one to eight copies of the third antigen binding portion. In some embodiments, the first antigen binding portion can be a bispecific antibody. In some embodiments, the first antigen binding portion is a monospecific full-length antibody or antigen binding fragment thereof, such as a Fab.

In some embodiments, the MABP comprises any one of 2, 3, 4, 5, 6, 7, 8, or more different antigen binding portions that each comprises an sdAb. Each sdAb may be directly fused to the first antigen binding portion, or fused to another sdAb, wherein the fused sdAb is further fused to the first antigen binding portion.

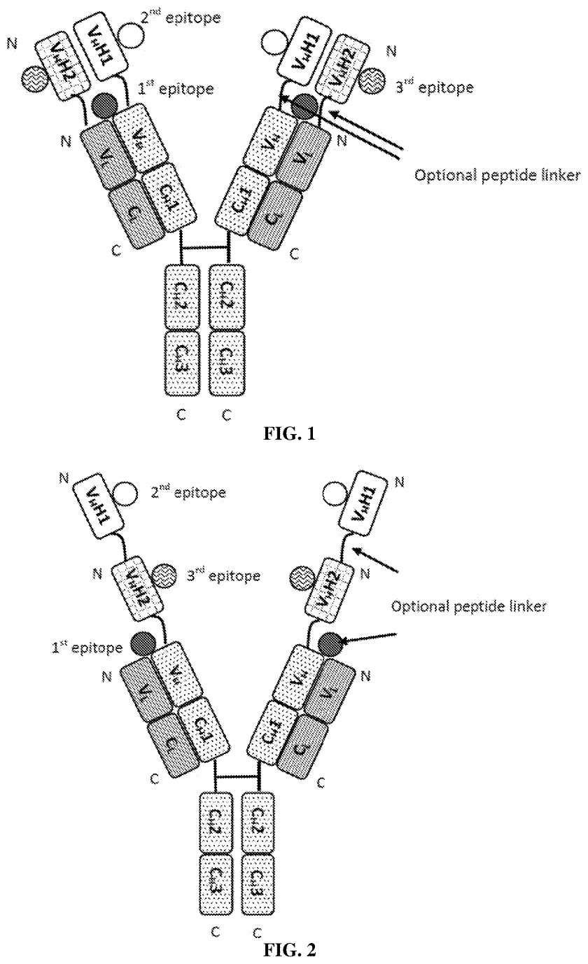

The MABPs may have any suitable number of valencies for each epitope, and any suitable number of specificity. In some embodiments, the MABP is bivalent, trivalent, tetravalent, pentavalent, hexavalent, or of higher valencies for the first epitope. In some embodiments, the MABP is monovalent, bivalent, trivalent, tetravalent, pentavalent, hexavalent, or of higher valencies for the second epitope. In some embodiments, the MABP is monovalent, bivalent, trivalent, tetravalent, pentavalent, hexavalent, or of higher valencies for the third epitope. In some embodiments, the MABP is trispecific. In some embodiments, the MABP is tetraspecific. In some embodiments, the MABP has more than four specificities. Exemplary trispecific antigen binding proteins (“TABPs”) are depicted in FIGS. 1-10.

In some embodiments, there is provided a MABP (e.g., TABP) comprising: (a) one or more copies (e.g., 1 or 2) of a first antigen binding portion comprising a heavy chain variable domain (V_{H}) and a light chain variable domain (V_{L}), wherein the V_{H} and V_{L} together form an antigen-binding site that specifically binds a first epitope, (b) one or more copies (e.g., 2) of a second antigen binding portion comprising an sdAb that specifically binds a second epitope, and (c) one or more copies (e.g., 2) of a third antigen binding portion comprising an sdAb that specifically binds a third epitope, wherein the first antigen binding portion, the second antigen binding portion, and the third antigen binding portion are fused to each other.

The first antigen binding portion, the second antigen binding portion, and the third antigen binding portion may be fused to each other in any suitable format. In some embodiments, each of the second antigen binding portion and the third antigen binding portion comprises a single polypeptide chain. In some embodiments, the first antigen binding portion comprises one or more (e.g., 2) heavy chains and one or more (e.g., 2) light chains. In some embodiments, the C terminus of the second antigen binding portion is fused to the N-terminus of at least one heavy chain of the first antigen binding portion, and the C-terminus of the third antigen binding portion is fused to the N-terminus of at least one light chain of the first antigen binding portion. In some embodiments, the C-terminus of second antigen binding portion is fused to the N-terminus of the third antigen binding portion, and the C-terminus of the third antigen binding portion is fused to the N-terminus of at least one heavy chain of the first antigen binding portion. In some embodiments, the C-terminus of second antigen binding portion is fused to the N-terminus of the third antigen binding portion, and the C-terminus of the third antigen binding portion is fused to the N-terminus of at least one light chain of the first antigen binding portion. In some embodiments, the C-terminus of the second antigen binding portion is fused to the N-terminus of at least one light chain of the first antigen binding portion, and the N-terminus of the third antigen binding portion is fused to the C-terminus of at least one light chain of the first antigen binding portion. In some embodiments, the C terminus of the second antigen binding portion is fused to the N-terminus of at least one heavy chain of the first antigen binding portion, and the N-terminus of the third antigen binding portion is fused to the C-terminus of at least one light chain of the first antigen binding portion. In some embodiments, the C terminus of the second antigen binding portion is fused to the N-terminus of at least one heavy chain of the first antigen binding portion, and the N-terminus of the third antigen binding portion is fused to the C-terminus of at least one heavy chain of the first antigen binding portion. In some embodiments, the C-terminus of the second antigen binding portion is fused to the N-terminus of at least one light chain of the first antigen binding portion, and the N-terminus of the third antigen binding portion is fused to the C-terminus of at least one heavy chain of the first antigen binding portion. In some embodiments, the C-terminus of the second antigen binding portion is fused to the C-terminus of at least one heavy chain of the first antigen binding portion, and the N-terminus of the third antigen binding portion is fused to the C-terminus of at least one light chain of the first antigen binding portion. In some embodiments, the C-terminus of the third antigen binding portion is fused to the N-terminus of the second antigen binding portion, and the N-terminus of the third antigen binding portion is fused to the C-terminus of at least one light chain of the first antigen binding portion. In some embodiments, the C terminus of the third antigen binding portion is fused to the N-terminus of the second antigen binding portion, and the N-terminus of the third antigen binding portion is fused to the C-terminus of at least one heavy chain of the first antigen binding portion.

In some embodiments, there is provided a MABP (e.g., TABP) comprising: (a) a first antigen binding portion comprising a heavy chain variable domain (V_{H}) and a light chain variable domain (V_{L}), wherein the V_{H} and V_{L} together form an antigen-binding site that specifically binds a first epitope, (b) a second antigen binding portion comprising a first sdAb that specifically binds a second epitope, wherein C-terminus of the first sdAb is fused to N-terminus of the V_{H} of the first antigen binding portion, and (c) a third antigen binding portion comprising a second sdAb that specifically binds a third epitope, wherein the C-terminus of the second sdAb is fused to N-terminus of the V_{L} of the first antigen binding portion. In some embodiments, the TABP comprises: (a) two chains of a first polypeptide each comprising from the N-terminus to the C-terminus: V_{H}H1-V_{H}-C_{H}1-C_{H}2-C_{H}3, and (b) two chains of a second polypeptide each comprising from the N-terminus to the C-terminus: V_{H}H2-V_{L}-C_{L}, wherein V_{H} and V_{L} together form an antigen-binding site that specifically binds a first epitope, V_{H}H1 is a first sdAb that specifically binds a second epitope, and V_{H}H2 is a second sdAb that specifically binds a third epitope. An example is shown in FIG. 1.

In some embodiments, there is provided a MABP (e.g., TABP) comprising: (a) a first antigen binding portion comprising a heavy chain variable domain (V_{H}) and a light chain variable domain (V_{L}), wherein the V_{H} and V_{L} together form an antigen-binding site that specifically binds a first epitope, (b) a second antigen binding portion comprising a first sdAb that specifically binds a second epitope, and (c) a third antigen binding portion comprising a second sdAb that specifically binds a third epitope, wherein the C-terminus of the first sdAb is fused to the N-terminus of the second sdAb, and the C-terminus of the second sdAb is fused to the N-terminus of the V_{H} of the first antigen binding portion. In some embodiments, the TABP comprises: (a) two chains of a first polypeptide each comprising from the N-terminus to the C-terminus: V_{H}H1-V_{H}H2-V_{H}-C_{H}1-C_{H}2-C_{H}3, and (b) two chains of a second polypeptide each comprising from the N-terminus to the C-terminus: V_{L}—C_{L}, wherein V_{H} and V_{L} together form an antigen-binding site that specifically binds a first epitope, V_{H}H1 is a first sdAb that specifically binds a second epitope, and V_{H}H2 is a second sdAb that specifically binds a third epitope. An example is shown in FIG. 2.

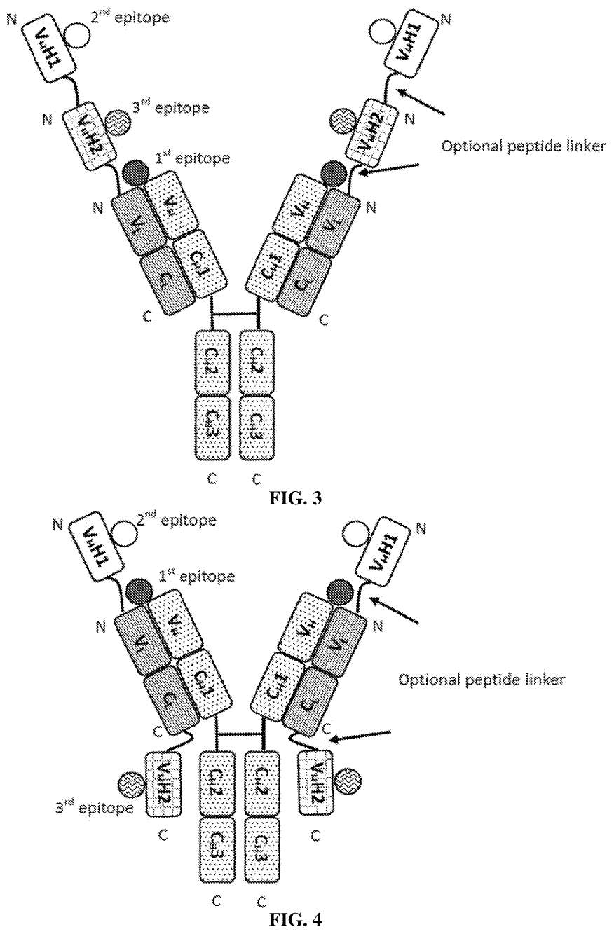

In some embodiments, there is provided a MABP (e.g., TABP) comprising: (a) a first antigen binding portion comprising a heavy chain variable domain (V_{H}) and a light chain variable domain (V_{L}), wherein the V_{H} and V_{L} together form an antigen-binding site that specifically binds a first epitope, (b) a second antigen binding portion comprising a first sdAb that specifically binds a second epitope, and (c) a third antigen binding portion comprising a second sdAb that specifically binds a third epitope, wherein the C-terminus of the first sdAb is fused to the N-terminus of the second sdAb, and the C-terminus of the second sdAb is fused to the N-terminus of the V_{L} of the first antigen binding portion. In some embodiments, the TABP comprises: (a) two chains of a first polypeptide each comprising from the N-terminus to the C-terminus: V_{H}—C_{H}1-C_{H}2-C_{H}3, and (b) two chains of a second polypeptide each comprising from the N-terminus to the C-terminus: V_{H}H1-V_{H}H2-V_{L}-C_{L}, wherein V_{H} and V_{L} together form an antigen-binding site that specifically binds a first epitope, V_{H}H1 is a first sdAb that specifically binds a second epitope, and V_{H}H2 is a second sdAb that specifically binds a third epitope. An example is shown in FIG. 3.

In some embodiments, there is provided a MABP (e.g., TABP) comprising: (a) a first antigen binding portion comprising a heavy chain comprising a heavy chain variable region (V_{H}) and a heavy chain constant region (C_{H}1) and a light chain comprising a light chain variable region (V_{L}) and a light chain constant region (C_{L}), wherein the V_{H} and V_{L} together form an antigen-binding site that specifically binds a first epitope, (b) a second antigen binding portion comprising a first sdAb that specifically binds a second epitope, and (c) a third antigen binding portion comprising a second sdAb that specifically binds a third epitope, wherein the C-terminus of the first sdAb is fused to the N-terminus of the V_{L} of the first antigen binding portion and the C-terminus of the C_{L} is fused to the N-terminus of the second sdAb. In some embodiments, the TABP comprises: (a) two chains of a first polypeptide each comprising from the N-terminus to the C-terminus: V_{H}—C_{H}1-C_{H}2-C_{H}3, and (b) two chains of a second polypeptide each comprising from the N-terminus to the C-terminus: V_{H}H1-V_{L}—C_{L}-V_{H}H2, wherein V_{H} and V_{L} together form an antigen-binding site that specifically binds a first epitope, V_{H}H1 is a first sdAb that specifically binds a second epitope, and V_{H}H2 is a second sdAb that specifically binds a third epitope. An example is shown in FIG. 4.

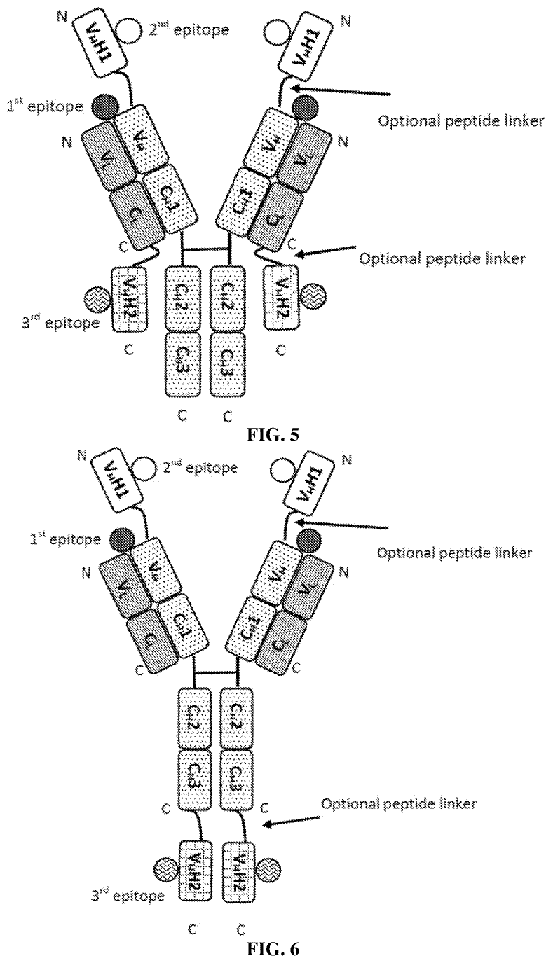

In some embodiments, there is provided a MABP (e.g., TABP) comprising: (a) a first antigen binding portion comprising a heavy chain comprising a heavy chain variable region (V_{H}) and a heavy chain constant region (C_{H}1) and a light chain comprising a light chain variable region (V_{L}) and a light chain constant region (C_{L}), wherein the V_{H} and V_{L} together form an antigen-binding site that specifically binds a first epitope, (b) a second antigen binding portion comprising a first sdAb that specifically binds a second epitope, and (c) a third antigen binding portion comprising a second sdAb that specifically binds a third epitope, wherein the C-terminus of the first sdAb is fused to the N-terminus of the V_{H} of the first antigen binding portion and the C-terminus of the C_{L} of the first antigen binding portion is fused to the N-terminus of the second sdAb. In some embodiments, the TABP comprises: (a) two chains of a first polypeptide each comprising from the N-terminus to the C-terminus: V_{H}H1-V_{H}-C_{H}1-C_{H}2-C_{H}3, and (b) two chains of a second polypeptide each comprising from the N-terminus to the C-terminus: V_{L}—C_{L}-V_{H}H2, wherein V_{H} and V_{L} together form an antigen-binding site that specifically binds a first epitope, V_{H}H1 is a first sdAb that specifically binds a second epitope, and V_{H}H2 is a second sdAb that specifically binds a third epitope. An example is shown in FIG. 5.

In some embodiments, there is provided a MABP (e.g., TABP) comprising: (a) a first antigen binding portion comprising a heavy chain comprising a heavy chain variable region (V_{H}) and heavy chain constant regions C_{H}1, C_{H}2 and C_{H}3, and a light chain comprising a light chain variable region (V_{L}) and a light chain constant region (C_{L}), wherein the V_{H} and V_{L} together form an antigen-binding site that specifically binds a first epitope, (b) a second antigen binding portion comprising a first sdAb that specifically binds a second epitope, and (c) a third antigen binding portion comprising a second sdAb that specifically binds a third epitope, wherein the C-terminus of the first sdAb is fused to the N-terminus of the V_{H} of the first antigen binding portion and the C-terminus of the C_{H}3 of the first antigen binding portion is fused to the N-terminus of the second sdAb. In some embodiments, the TABP comprises: (a) two chains of a first polypeptide each comprising from the N-terminus to the C-terminus: V_{H}H1-V_{H}-C_{H}1-C_{H}2-C_{H}3-V_{H}H2, and (b) two chains of a second polypeptide each comprising from the N-terminus to the C-terminus: V_{L}—C_{L}, wherein V_{H} and V_{L} together form an antigen-binding site that specifically binds a first epitope, V_{H}H1 is a first sdAb that specifically binds a second epitope, and V_{H}H2 is a second sdAb that specifically binds a third epitope. An example is shown in FIG. 6.

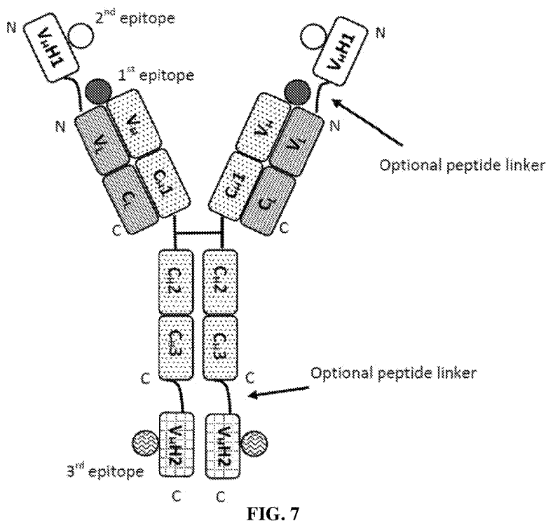

In some embodiments, there is provided a MABP (e.g., TABP) comprising: (a) a first antigen binding portion comprising a heavy chain comprising a heavy chain variable region (V_{H}) and heavy chain constant regions C_{H}1, C_{H}2 and C_{H}3, and a light chain comprising a light chain variable region (V_{L}) and a light chain constant region (C_{L}), wherein the V_{H} and V_{L} together form an antigen-binding site that specifically binds a first epitope, (b) a second antigen binding portion comprising a first sdAb that specifically binds a second epitope, and (c) a third antigen binding portion comprising a second sdAb that specifically binds a third epitope, wherein the C-terminus of the first sdAb is fused to the N-terminus of the V_{L} of the first antigen binding portion and the C-terminus of the C_{H}3 of the first antigen binding portion is fused to the N-terminus of the second sdAb. In some embodiments, the TABP comprises: (a) two chains of a first polypeptide each comprising from the N-terminus to the C-terminus: V_{H}—C_{H}1-C_{H}2-C_{H}3-V_{H}H2, and (b) two chains of a second polypeptide each comprising from the N-terminus to the C-terminus: V_{H}H1-V_{L}-C_{L}, wherein V_{H} and V_{L} together form an antigen-binding site that specifically binds a first epitope, V_{H}H1 is a first sdAb that specifically binds a second epitope, and V_{H}H2 is a second sdAb that specifically binds a third epitope. An example is shown in FIG. 7.

In some embodiments, there is provided a MABP (e.g., TABP) comprising: (a) a first antigen binding portion comprising a heavy chain comprising a heavy chain variable region (V_{H}) and heavy chain constant regions C_{H}1, C_{H}2 and C_{H}3, and a light chain comprising a light chain variable region (V_{L}) and a light chain constant region (C_{L}), wherein the V_{H} and V_{L} together form an antigen-binding site that specifically binds a first epitope, (b) a second antigen binding portion comprising a first sdAb that specifically binds a second epitope, and (c) a third antigen binding portion comprising a second sdAb that specifically binds a third epitope, wherein the N-terminus of the first sdAb is fused to the C-terminus of the C_{H}3 of the first antigen binding portion and the C-terminus of the C_{L} of the first antigen binding portion is fused to the N-terminus of the second sdAb. In some embodiments, the TABP comprises: (a) two chains of a first polypeptide each comprising from the N-terminus to the C-terminus: V_{H}—C_{H}1-C_{H}2-C_{H}3-V_{H}H1, and (b) two chains of a second polypeptide each comprising from the N-terminus to the C-terminus: V_{L}—C_{L}-V_{H}H2, wherein V_{H} and V_{L} together form an antigen-binding site that specifically binds a first epitope, V_{H}H1 is a first sdAb that specifically binds a second epitope, and V_{H}H2 is a second sdAb that specifically binds a third epitope. An example is shown in FIG. 8.

In some embodiments, there is provided a MABP (e.g., TABP) comprising: (a) a first antigen binding portion comprising a heavy chain comprising a heavy chain variable region (V_{H}) and a heavy chain constant region (C_{H}1), and a light chain comprising a light chain variable region (V_{L}) and a light chain constant region (C_{L}), wherein the V_{H} and V_{L} together form an antigen-binding site that specifically binds a first epitope, (b) a second antigen binding portion comprising a first sdAb that specifically binds a second epitope, and (c) a third antigen binding portion comprising a second sdAb that specifically binds a third epitope, wherein the N-terminus of the second sdAb is fused to the C-terminus of the C_{L} of the first antigen binding portion and the C-terminus of the second sdAb is fused to the N-terminus of the first sdAb. In some embodiments, the TABP comprises: (a) two chains of a first polypeptide each comprising from the N-terminus to the C-terminus: V_{H}—C_{H}1-C_{H}2-C_{H}3, and (b) two chains of a second polypeptide each comprising from the N-terminus to the C-terminus: V_{L}—C_{L}—V_{H}H2-V_{H}H1, wherein V_{H} and V_{L} together form an antigen-binding site that specifically binds a first epitope, V_{H}H1 is a first sdAb that specifically binds a second epitope, and V_{H}H2 is a second sdAb that specifically binds a third epitope. An example is shown in FIG. 9.

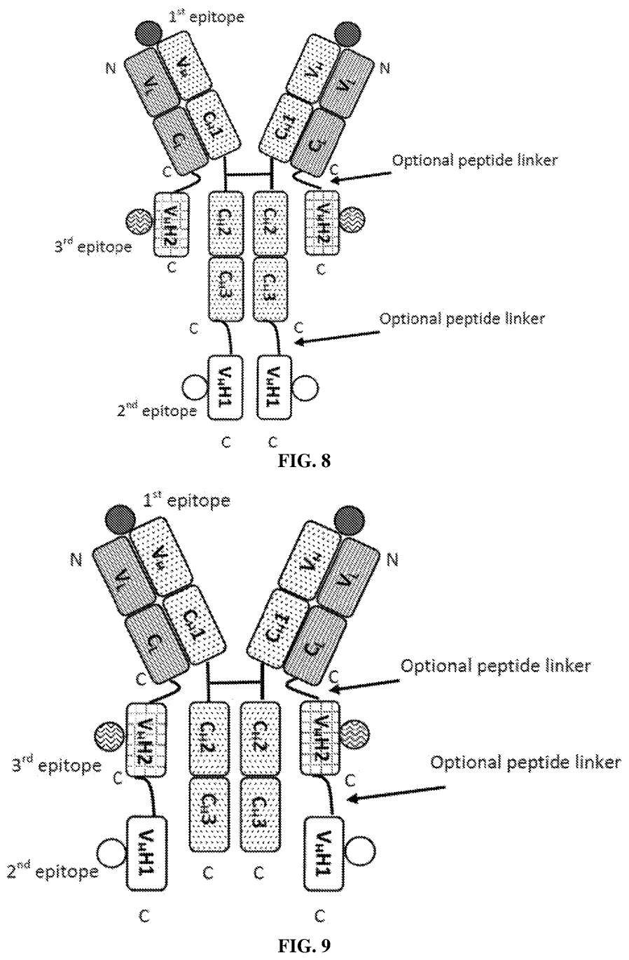

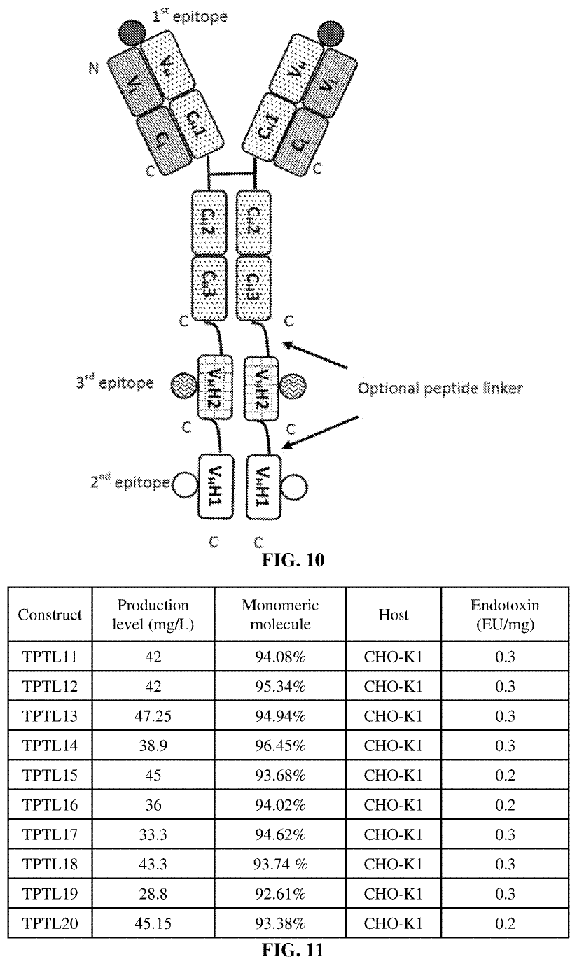

In some embodiments, there is provided a MABP (e.g., TABP) comprising: (a) a first antigen binding portion comprising a heavy chain comprising a heavy chain variable region (V_{H}) and heavy chain constant regions C_{H}1, C_{H}2 and C_{H}3, and a light chain comprising a light chain variable region (V_{L}) and a light chain constant region (C_{L}), wherein the V_{H} and V_{L} together form an antigen-binding site that specifically binds a first epitope, (b) a second antigen binding portion comprising a first sdAb that specifically binds a second epitope, and (c) a third antigen binding portion comprising a second sdAb that specifically binds a third epitope, wherein the N-terminus of the second sdAb is fused to the C-terminus of the C_{H}3 of the first antigen binding portion and the C-terminus of the second sdAb is fused to the N-terminus of the first sdAb. In some embodiments, the TABP comprises: (a) two chains of a first polypeptide each comprising from the N-terminus to the C-terminus: V_{H}—C_{H}I-C_{H}2-C_{H}3-V_{H}H2-V_{H}H1, and (b) two chains of a second polypeptide each comprising from the N-terminus to the C-terminus: V_{L}—C_{L} wherein V_{H} and V_{L} together form an antigen-binding site that specifically binds a first epitope, V_{H}H1 is a first sdAb that specifically binds a second epitope, and V_{H}H2 is a second sdAb that specifically binds a third epitope. An example is shown in FIG. 10.

### Epitopes and Antigens

Any of the MABPs described herein can specifically bind at least three different epitopes. The at least three different epitopes recognized can be located on the same antigen, or on different antigens. In some embodiments, the antigens are cell surface molecules. In some embodiments, the antigens are extracellular molecules.

In some embodiments, the first epitope, the second epitope and/or the third epitope is an immune checkpoint molecule. In some embodiments, the immune checkpoint molecule is a stimulatory immune checkpoint molecule. Exemplary stimulatory immune checkpoint molecules include, but are not limited to, CD28, OX40, ICOS, GITR, 4-1BB, CD27, CD40, CD3, HVEM, and TCR (e.g., MHC class I or class II molecules). In some embodiments, the immune checkpoint molecule is an inhibitory immune checkpoint molecule. Exemplary inhibitory immune checkpoint molecules include, but are not limited to, CTLA-4, TIM-3, A2a Receptor, LAG-3, TIGIT, BTLA, KIR, PD-1, IDO, CD47, and ligands thereof such as B7.1, B7.2, PD-L1, PD-L2, HVEM, B7-H4, NKTR-218, and SIRP-alpha receptor.

Thus, in some embodiments, there is provided a MABP (e.g., TABP) comprising: (a) a first antigen binding portion comprising a heavy chain variable domain (V_{H}) and a light chain variable domain (V_{L}), wherein the V_{H} and V_{L} together form an antigen-binding site that specifically binds a first immune checkpoint molecule, (b) a second antigen binding portion comprising a sdAb that specifically binds a second immune checkpoint molecule, and (c) a third antigen binding portion comprising a second sdAb that specifically binds a third immune checkpoint molecule, wherein the first antigen binding portion, the second antigen binding portion, and the third antigen binding portion are fused to each other. In some embodiments, the first immune checkpoint molecule, the second immune checkpoint molecule, and/or the third immune checkpoint molecule is selected from the group consisting of PD-1, PD-L1, PD-L2, CTLA-4, B7-H3, TIM-3, LAG-3, TIGIT, VISTA, ICOS, 4-1BB, OX40, GITR, and CD40. In some embodiments, the first sdAb and/or the second sdAb is a V_{H}H. In some embodiments, the first antigen binding portion comprises a heavy chain comprising the V_{H} and a light chain comprising the V_{L}. In some embodiments, the first antigen binding region is a full-length antibody consisting of two heavy chains and two light chains. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG4 Fc.

In some embodiments, there is provided a MABP (e.g., TABP) comprising: (a) a first antigen binding portion comprising a heavy chain variable domain (V_{H}) and a light chain variable domain (V_{L}), wherein the V_{H} and V_{L} together form an antigen-binding site that specifically binds PD-1, (b) a second antigen binding portion comprising a first sdAb that specifically binds TIGIT, and (c) a third antigen binding portion comprising a second sdAb that specifically binds LAG-3, wherein the first antigen binding portion, the second antigen binding portion, and the third antigen binding portion are fused to each other. In some embodiments, the first sdAb and/or the second sdAb is a V_{H}H. In some embodiments, the first antigen binding portion comprises a heavy chain comprising the V_{H} and a light chain comprising the V_{L}. In some embodiments, the first antigen binding region is a full-length antibody consisting of two heavy chains and two light chains, e.g., pembrolizumab. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG4 Fc.

In some embodiments, the first epitope, the second epitope and/or the third epitope is a cell surface antigen. In some embodiments, the cell surface antigen is an antigen on immune effector cells, such as T cells (e.g., helper T cells, cytotoxic T cells, memory T cells, etc.), B cells, macrophages, and Natural Killer (NK) cells. In some embodiments, the cell surface antigen is a T cell surface antigen, such as CD3.

In some embodiments, the cell surface antigen is a tumor antigen. Tumor antigens are proteins that are produced by tumor cells that can elicit an immune response, particularly T-cell mediated immune responses. The selection of the targeted antigen described herein will depend on the particular type of cancer to be treated. Exemplary tumor antigens include, for example, a glioma-associated antigen, carcinoembryonic antigen (CEA), (3-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In some embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD 19, CD20 and CD37 are other candidates for target antigens in B-cell lymphoma.

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include the following: Differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23HI, PSA, TAG-72, CA 19-9, CA 72-4, CAM 17.1, NuMa, K-ras, beta-Catenin, CDK4, Mum-1, p15, p16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

Thus, in some embodiments, there is provided a MABP (e.g., TABP) comprising: (a) a first antigen binding portion comprising a heavy chain variable domain (V_{H}) and a light chain variable domain (V_{L}), wherein the V_{H} and V_{L} together form an antigen-binding site that specifically binds a first tumor antigen, (b) a second antigen binding portion comprising a first sdAb that specifically binds a second tumor antigen, and (c) a third antigen binding portion comprising a second sdAb that specifically binds a third tumor antigen, wherein the first antigen binding portion, the second antigen binding portion, and the third antigen binding portion are fused to each other. In some embodiments, the first tumor antigen, the second tumor antigen and/or the third tumor antigen is selected from the group consisting of HER2, BRAF, EGFR, VEGFR2, CD20, RANKL, CD38, and CD52. In some embodiments, the first sdAb and/or the second sdAb is a V_{H}H. In some embodiments, the first antigen binding portion comprises a heavy chain comprising the V_{H} and a light chain comprising the V_{L}. In some embodiments, the first antigen binding region is a full-length antibody consisting of two heavy chains and two light chains. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG1 Fc.

In some embodiments, there is provided a MABP (e.g., TABP) comprising: (a) a first antigen binding portion comprising a heavy chain variable domain (V_{H}) and a light chain variable domain (V_{L}), wherein the V_{H} and V_{L} together form an antigen-binding site that specifically binds a first tumor antigen, (b) a second antigen binding portion comprising a first sdAb that specifically binds a cell surface antigen on an immune effector cell (such as T cell), and (c) a third antigen binding portion comprising a second sdAb that specifically binds a second tumor antigen, wherein the first antigen binding portion, the second antigen binding portion, and the third antigen binding portion are fused to each other. In some embodiments, the first tumor antigen and/or the second tumor antigen is selected from the group consisting of HER2, BRAF, EGFR, VEGFR2, CD20, RANKL, CD38, and CD52. In some embodiments, the first sdAb and/or the second sdAb is a V_{H}H. In some embodiments, the first antigen binding portion comprises a heavy chain comprising the V_{H} and a light chain comprising the V_{L}. In some embodiments, the first antigen binding region is a full-length antibody consisting of two heavy chains and two light chains. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG1 Fc or IgG4 Fc.

In some embodiments, there is provided a MABP (e.g., TABP) comprising: (a) a first antigen binding portion comprising a heavy chain variable domain (V_{H}) and a light chain variable domain (V_{L}), wherein the V_{H} and V_{L} together form an antigen-binding site that specifically binds HER-2, (b) a second antigen binding portion comprising a first sdAb that specifically binds CD3, and (c) a third antigen binding portion comprising a second sdAb that specifically binds EGFR, wherein the first antigen binding portion, the second antigen binding portion, and the third antigen binding portion are fused to each other. In some embodiments, the first sdAb and/or the second sdAb is a V_{H}H. In some embodiments, the first antigen binding portion comprises a heavy chain comprising the V_{H} and a light chain comprising the V_{L}. In some embodiments, the first antigen binding region is a full-length antibody consisting of two heavy chains and two light chains, e.g., trastuzumab. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG1 Fc or IgG4 Fc.

In some embodiments, the first epitope, the second epitope, and/or the third epitope is a pro-inflammatory molecule. “Pro-inflammatory molecule” refers to any molecule produced or expressed by an immune cell (such as monocytes, macrophages, lymphocytes and leukocytes) that up-regulates inflammatory reactions. In some embodiments, the pro-inflammatory molecule is a pro-inflammatory cytokine, such as lymphokine, monokine, chemokine, or interleukin. Exemplary pro-inflammatory molecules include, but are not limited to, IL-1β, TNF-α, IL-6, IL-6R, IL-5, IL-17A, IL-17F, IL-23, IL-22, IL-21, IL-12, and eotaxin-1 (i.e., CCL11).

Thus, in some embodiments, there is provided a MABP (e.g., TABP) comprising: (a) a first antigen binding portion comprising a heavy chain variable domain (V_{H}) and a light chain variable domain (V_{L}), wherein the V_{H} and V_{L} together form an antigen-binding site that specifically binds a first pro-inflammatory molecule, (b) a second antigen binding portion comprising a first sdAb that specifically binds a second pro-inflammatory molecule, and (c) a third antigen binding portion comprising a second sdAb that specifically binds a third pro-inflammatory molecule, wherein the first antigen binding portion, the second antigen binding portion, and the third antigen binding portion are fused to each other. In some embodiments, the first pro-inflammatory molecule, the second pro-inflammatory molecule and/or the third pro-inflammatory molecule is selected from the group consisting of IL-1β, TNF-α, IL-6, IL-6R, IL-5, IL-17A, IL-17F, IL-23, IL-22, IL-21, IL-12, and eotaxin-1. In some embodiments, the first sdAb and/or the second sdAb is a V_{H}H. In some embodiments, the first antigen binding portion comprises a heavy chain comprising the V_{H} and a light chain comprising the V_{L}. In some embodiments, the first antigen binding region is a full-length antibody consisting of two heavy chains and two light chains. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG1 Fc.

In some embodiments, there is provided a MABP (e.g., TABP) comprising: (a) a first antigen binding portion comprising a heavy chain variable domain (V_{H}) and a light chain variable domain (V_{L}), wherein the V_{H} and V_{L} together form an antigen-binding site that specifically binds TNF-α, (b) a second antigen binding portion comprising a first sdAb that specifically binds IL-17A, and (c) a third antigen binding portion comprising a second sdAb that specifically binds IL-17F, wherein the first antigen binding portion, the second antigen binding portion, and the third antigen binding portion are fused to each other. In some embodiments, the first sdAb and/or the second sdAb is a V_{H}H. In some embodiments, the first antigen binding portion comprises a heavy chain comprising the V_{H} and a light chain comprising the V_{L}. In some embodiments, the first antigen binding region is a full-length antibody consisting of two heavy chains and two light chains, e.g., adalimumab. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG1 Fc.

In some embodiments, the first epitope, the second epitope and/or the third epitope is an angiogenic factor, such as Ang2, VEGF and DLL4.

Thus, in some embodiments, there is provided a MABP (e.g., TABP) comprising: (a) a first antigen binding portion comprising a heavy chain variable domain (V_{H}) and a light chain variable domain (V_{L}), wherein the V_{H} and V_{L} together form an antigen-binding site that specifically binds a first angiogenic factor, (b) a second antigen binding portion comprising a first sdAb that specifically binds a second angiogenic factor, and (c) a third antigen binding portion comprising a second sdAb that specifically binds a third angiogenic factor, wherein the first antigen binding portion, the second antigen binding portion, and the third antigen binding portion are fused to each other. In some embodiments, the first sdAb and/or the second sdAb is a V_{H}H. In some embodiments, the first antigen binding portion comprises a heavy chain comprising the V_{H} and a light chain comprising the V_{L}. In some embodiments, the first antigen binding region is a full-length antibody consisting of two heavy chains and two light chains. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG1 Fc.

In some embodiments, there is provided a MABP (e.g., TABP) comprising: (a) a first antigen binding portion comprising a heavy chain variable domain (V_{H}) and a light chain variable domain (V_{L}), wherein the V_{H} and V_{L} together form an antigen-binding site that specifically binds Ang-2, (b) a second antigen binding portion comprising a first sdAb that specifically binds VEGF, and (c) a third antigen binding portion comprising a second sdAb that specifically binds DLL4, wherein the first antigen binding portion, the second antigen binding portion, and the third antigen binding portion are fused to each other. In some embodiments, the first sdAb and/or the second sdAb is a V_{H}H. In some embodiments, the first antigen binding portion comprises a heavy chain comprising the V_{H} and a light chain comprising the V_{L}. In some embodiments, the first antigen binding region is a full-length antibody consisting of two heavy chains and two light chains, e.g., LC10. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG1 Fc.

### Fusion Polypeptides

The first antigen binding portion, the second antigen binding portion, and the third antigen binding portion of the MABP are directly or indirectly fused (i.e., covalently linked) to each other. Thus, the MABPs of the present application comprise one or more fusion polypeptides. Each fusion polypeptide may comprise the second antigen binding portion and/or the third antigen binding portion, and a polypeptide from the first antigen binding portion.

The various antigen binding portions can be fused chemically, by a single chemical bond (such as peptide bond), or via a peptide linker. The second antigen binding portion and the third antigen binding portion may each be fused at either the N-terminus or the C-terminus of any one (including each) polypeptide of the first antigen binding portion. The second antigen binding portion and the third antigen binding portion may also be fused directly to each other, and the fused sdAbs may be fused at either the N-terminus or the C-terminus of any one (including each) polypeptide of the first antigen binding portion. The fusion polypeptides may be obtained either recombinantly or chemically.

Thus, in some embodiments, there is provided a MABP (e.g., TABP) comprising: (a) a full-length antibody consisting of two heavy chains and two light chains, wherein the full-length antibody specifically recognizes a first epitope; (b) a first sdAb that specifically recognizes a second epitope; and (c) a second sdAb that specifically recognizes a third epitope, wherein the full-length antibody, the first sdAb and the second sdAb are fused to each other. In some embodiments, the full-length antibody is a full-length monoclonal antibody consisting of two identical heavy chains and two identical light chains. In some embodiments, the first sdAb and/or the second sdAb is a V_{H}H. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG4 Fc or IgG1 Fc.

In some embodiments, the MABP (e.g., TABP) comprises: (1) a first polypeptide comprising from the N-terminus to the C-terminus: the first sdAb, an optional peptide linker, and a heavy chain of the first antigen binding portion; and (2) a second polypeptide comprising from the N-terminus to the C-terminus: the second sdAb, an optional peptide linker, and a light chain of the first antigen binding portion. See, for example, FIG. 1.

In some embodiments, the MABP (e.g., TABP) comprises: (1) a first polypeptide comprising from the N-terminus to the C-terminus: the first sdAb, an optional peptide linker, the second sdAb, an optional peptide linker and a heavy chain of the first antigen binding portion, and (2) a second polypeptide comprising from the N-terminus to the C-terminus: a light chain of the first antigen binding portion. See, for example, FIG. 2.

In some embodiments, the MABP (e.g., TABP) comprises: (1) a first polypeptide comprising from the N-terminus to the C-terminus: a heavy chain of the first antigen binding portion; and (2) a second polypeptide comprising from the N-terminus to the C-terminus: the first sdAb, an optional peptide linker, the second sdAb, an optional peptide linker and a light chain of the first antigen binding portion. See, for example, FIG. 3.

In some embodiments, the MABP (e.g., TABP) comprises: (1) a first polypeptide comprising from the N-terminus to the C-terminus: a heavy chain of the first antigen binding portion, an optional peptide linker, and the first sdAb; and (2) a second polypeptide comprising from the N-terminus to the C-terminus: a light chain of the first antigen binding portion, an optional peptide linker, and the second sdAb. See, for example, FIG. 4.

In some embodiments, the MABP (e.g., TABP) comprises: (1) a first polypeptide comprising from the N-terminus to the C-terminus: the first sdAb, an optional peptide linker, a heavy chain of the first antigen binding portion; and (2) a second polypeptide comprising from the N-terminus to the C-terminus: a light chain of the first antigen binding portion, an optional peptide linker and the second sdAb. See, for example, FIG. 5.

In some embodiments, the MABP (e.g., TABP) comprises: (1) a first polypeptide comprising from the N-terminus to the C-terminus: the first sdAb, an optional peptide linker, a heavy chain of the first antigen binding portion, an optional peptide linker, the second sdAb; and (2) a second polypeptide comprising from the N-terminus to the C-terminus a light chain of the first antigen binding portion, See, for example, FIG. 6.

In some embodiments, the MABP (e.g., TABP) comprises: (1) a first polypeptide comprising from the N-terminus to the C-terminus: a heavy chain of the first antigen binding portion, an optional peptide linker, the first sdAb; and (2) a second polypeptide comprising from the N-terminus to the C-terminus: the second sdAb, an optional peptide linker, a light chain of the first antigen binding portion. See, for example, FIG. 7.

In some embodiments, the MABP (e.g., TABP) comprises: (1) a first polypeptide comprising from the N-terminus to the C-terminus: a heavy chain of the first antigen binding portion, an optional peptide linker, the first sdAb, and (2) a second polypeptide comprising from the N-terminus to the C-terminus: a light chain of the first antigen binding portion, an optional peptide linker and the second sdAb. See, for example, FIG. 8.

In some embodiments, the MABP (e.g., TABP) comprises: (1) a first polypeptide comprising from the N-terminus to the C-terminus: a heavy chain of the first antigen binding portion; and (2) a second polypeptide comprising from the N-terminus to the C-terminus: a light chain of the first antigen binding portion, an optional peptide linker, the second sdAb, an optional peptide linker and the first sdAb. See, for example, FIG. 9.

In some embodiments, the MABP (e.g., TABP) comprises: (1) a first polypeptide comprising from the N-terminus to the C-terminus: a heavy chain of the first antigen binding portion, an optional peptide linker, the second sdAb, an optional peptide linker and the first sdAb; and (2) a second polypeptide comprising from the N-terminus to the C-terminus: a light chain of the first antigen binding portion. See, for example, FIG. 10.

In some embodiments, the first antigen binding portion is a full-length antibody consisting of two heavy chains and two light chains. In some embodiments, the MABP comprises two chains of the first polypeptide and two chains of the second polypeptide.

The MABPs described herein may comprise one or more peptide linkers situated between the various antigen binding portions. In some embodiments, the various antigen binding portions are directly fused to each other without a peptide linker disposed there between.

The peptide linkers connecting different antigen binding portions may be the same or different. Each peptide linker can be optimized individually. The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50 or more amino acids long. In some embodiments, the peptide linker is no more than about any of 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, or about 1 amino acid to about 50 amino acids.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. Exemplary flexible linkers include glycine polymers (G)., glycine-serine polymers (including, for example, (GS). (SEQ ID NO: 4), (GSGGS)ₙ (SEQ ID NO: 5) and (GGGS). (SEQ ID NO: 6), where n is an integer of at least one), glycine-alanine polymers, alanine-serine polymers, and other flexible linkers known in the art. In some embodiments, the peptide linker comprises the amino acid sequence GGGGSGGGS (SEQ ID NO: 1). In some embodiments, the peptide linker comprises the amino acid sequence GGGGSGGGGSGGGGS (SEQ ID NO: 2). In some embodiments, the peptide linker comprises the hinge region of an IgG, such as the hinge region of human IgG1. In some embodiments, the peptide linker comprises the amino acid sequence EPKSCDKTHTCPPCP (SEQ ID NO: 7). In some embodiments, the peptide linker comprises a modified sequence derived from the hinge region of an IgG, such as the hinge region of human IgG1. For example, one or more cysteines in the hinge region of an IgG may be replaced with a serine. In some embodiments, the peptide linker comprises the amino acid sequence EPKSSDKTHTSPPSP (SEQ ID NO: 3).

In some embodiments, the various antigen binding portions are fused to each other chemically. For example, the antigen binding portions may be conjugated using one or more reactive sites via a linking group. Reactive sites in polypeptides that are useful for chemical conjugation are well known in the art, including, but not limited to primary amino groups present on amino acid residue such as the epsilon amino group of lysine, and the alpha amino group of N-terminal amino acids, thiol groups in cysteine residues, the carboxylic group of the C-terminal amino acids, and carbohydrate groups in glycosylated antibodies. In some embodiments, the reactive site is introduced into the second antigen binding portion or the first antigen binding portion by site-directed mutagenesis, incorporation of selenocysteines or unnatural amino acids, incorporation of bifunctional linkers (such as bis-alkylating reagents), and/or glycoengineering. In some embodiments, one or more primary amino groups of a polypeptide can be converted to a thiol-containing group (e.g., from a cysteine or homocysteine residue), an electrophilic unsaturated group such as a maleimide group, or halogenated group such as a bromoacetyl group, for conjugation to thiol reactive polypeptides. Any linking groups and conjugation methods known in the art can be used to chemically fuse the second antigen binding portion to the first antigen binding portion. In some embodiments, the conjugation can be achieved, for example, by using succinimide esters (such as succinimidyl 44N-maleimidomethylicyclohexane-1-carboxylate (SMCC), or N-maleimidobenzoyl-N-hydroxysuccinimide ester (MBS)), glutaraldehyde, carbodiimide (such as 1-ethyl-3-(3-dimethylaminopropyl) carbodiimide (EDCI)), benzidine (BDB), periodate, or isothiocyanate (such as N-acetyl homocysteine thiolactone (NAHT)).

### Exemplary Trispecific Antigen Binding Proteins (TABPS)

In some embodiments, there is provided a trispecific antigen binding protein (TABP) comprising: (a) a first antigen binding portion comprising a heavy chain variable domain (V_{H}) and a light chain variable domain (V_{L}), wherein the V_{H} and V_{L} together form an antigen-binding site that specifically binds PD-1, (b) a second antigen binding portion comprising a first sdAb that specifically binds TIGIT, and (c) a third antigen binding portion comprising a second sdAb that specifically binds LAG-3, wherein the first antigen binding portion, the second antigen binding portion, and the third antigen binding portion are fused to each other. In some embodiments, the first antigen binding portion comprises a V_{H} domain comprising the amino acid sequence of SEQ ID NO: 10 and a V_{L} domain comprising the amino acid sequence of SEQ ID NO: 11. In some embodiments, the first antigen binding portion comprises a heavy chain comprising the amino acid sequence of SEQ ID NO: 8. In some embodiments, the first antigen binding portion comprises a light chain comprising the amino acid sequence of SEQ ID NO: 9. In some embodiments, the first sdAb comprises a V_{H}H compring CDR1, CDR2, and CDR3 of the amino acid sequence of SEQ ID NO: 31, or a variant thereof comprising up to about 3 (such as about any of 1, 2, or 3) amino acid substitutions. In some embodiments, the second sdAb comprises a V_{H}H compring CDR1, CDR2, and CDR3 of the amino acid sequence of SEQ ID NO: 32, or a variant thereof comprising up to about 3 (such as about any of 1, 2, or 3) amino acid substitutions. In some embodiments, the first sdAb comprises a V_{H}H of the amino acid sequence of SEQ ID NO: 31, or a variant thereof comprising up to about 3 (such as about any of 1, 2, or 3) amino acid substitutions. In some embodiments, the second sdAb comprises a V_{H}H of the amino acid sequence of SEQ ID NO: 32, or a variant thereof comprising up to about 3 (such as about any of 1, 2, or 3) amino acid substitutions. In some embodiments, the first sdAb comprises a V_{H}H of the amino acid sequence of SEQ ID NO: 31. In some embodiments, the second sdAb comprises a V_{H}H of the amino acid sequence of SEQ ID NO: 32.

In some embodiments, there is provided a trispecific antigen binding protein (TABP) comprising: (a) a first antigen binding portion comprising a heavy chain variable domain (V_{H}) and a light chain variable domain (V_{L}), wherein the V_{H} and V_{L} together form an antigen-binding site that specifically binds PD-1, (b) a second antigen binding portion comprising a first sdAb that specifically binds TIGIT, and (c) a third antigen binding portion comprising a second sdAb that specifically binds LAG-3, wherein the first antigen binding portion comprises a V_{H} domain comprising the amino acid sequence of SEQ ID NO: 10 and a V_{L} domain comprising the amino acid sequence of SEQ ID NO: 11, the first sdAb comprises a V_{H}H of the amino acid sequence of SEQ ID NO: 31, or a variant thereof comprising up to about 3 (such as about any of 1, 2, or 3) amino acid substitutions, the second sdAb comprises a V_{H}H of the amino acid sequence of SEQ ID NO: 32, or a variant thereof comprising up to about 3 (such as about any of 1, 2, or 3) amino acid substitutions; wherein the first antigen binding portion, the second antigen binding portion, and the third antigen binding portion are fused to each other.

In some embodiments, there is provided a trispecific antigen binding protein (TABP) comprising: (a) a first antigen binding portion comprising a heavy chain variable domain (V_{H}) and a light chain variable domain (V_{L}), wherein the V_{H} and V_{L} together form an antigen-binding site that specifically binds PD-1, (b) a second antigen binding portion comprising a first sdAb that specifically binds TIGIT, and (c) a third antigen binding portion comprising a second sdAb that specifically binds LAG-3, wherein the first antigen binding portion comprises a V_{H} domain comprising the amino acid sequence of SEQ ID NO: 10 and a V_{L} domain comprising the amino acid sequence of SEQ ID NO: 11, the first sdAb comprises a V_{H}H of the amino acid sequence of SEQ ID NO: 31, the second sdAb comprises a V_{H}H of the amino acid sequence of SEQ ID NO: 32; wherein the first antigen binding portion, the second antigen binding portion, and the third antigen binding portion are fused to each other.

In some embodiments, a trispecific antigen binding protein (TABP) comprises: (1) a first polypeptide comprising the amino acid sequence of any one of SEQ ID NOs: 16, 18, 20, 23, 25, 26, 28, or a variant thereof comprising up to about 5 (such as about any of 1, 2, 3, 4, or 5) amino acid substitutions; and (2) a second polypeptide comprising the amino acid sequence of any one of SEQ ID NOs:15, 17, 19, 21, 22, 24, 27, or a variant thereof comprising up to about 5 (such as about any of 1, 2, 3, 4, or 5) amino acid substitutions. In some embodiments, a trispecific antigen binding protein (TABP) comprises: (1) a first polypeptide comprising the amino acid sequence of any one of SEQ ID NOs: 16, 18, 20, 23, 25, 26 or 28; and (2) a second polypeptide comprising the amino acid sequence of any one of SEQ ID NOs:15, 17, 19, 21, 22, 24 or 27.

In some embodiments, a trispecific antigen binding protein (TABP) comprises: (1) a first polypeptide comprising from the N-terminus to the C-terminus: an anti-TIGIT V_{H}H comprising the amino acid sequence of SEQ ID NO: 31, or a variant thereof comprising up to about 3 (such as about any of 1, 2, or 3) amino acid substitutions, a peptide linker comprising the amino acid sequence of any one of SEQ ID NOs: 1-7, and a heavy chain of an anti-PD-1 antibody comprising the amino acid sequence of SEQ ID NO:8; and (2) a second polypeptide comprising from the N-terminus to the C-terminus: an anti-LAG-3 V_{H}H comprising the amino acid sequence of SEQ ID NO: 32, or a variant thereof comprising up to about 3 (such as about any of 1, 2, or 3) amino acid substitutions, a peptide linker comprising the amino acid sequence of any one of SEQ ID NOs: 1-7, and a light chain of an anti-PD-1 antibody comprising the amino acid sequence of SEQ ID NO:9. In some embodiments, a trispecific antigen binding protein (TABP) comprises: (1) a first polypeptide comprising from the N-terminus to the C-terminus: an anti-TIGIT V_{H}H comprising the amino acid sequence of SEQ ID NO: 31, a peptide linker comprising the amino acid sequence of any one of SEQ ID NOs: 1-3, or 7, and a heavy chain of an anti-PD-1 antibody comprising the amino acid sequence of SEQ ID NO:8; and (2) a second polypeptide comprising from the N-terminus to the C-terminus: an anti-LAG-3 V_{H}H comprising the amino acid sequence of SEQ ID NO: 32, a peptide linker comprising the amino acid sequence of any one of SEQ ID NOs: 1-3, or 7, and a light chain of an anti-PD-1 antibody comprising the amino acid sequence of SEQ ID NO:9. In some embodiments, a trispecific antigen binding protein (TABP) comprises: (1) a first polypeptide comprising from the N-terminus to the C terminus: an anti-TIGIT V_{H}H, a peptide linker and a heavy chain of an anti-PD-1 antibody, and the first polypeptide comprising the amino acid sequence of SEQ ID NO:16, or a variant thereof comprising up to about 5 (such as about any of 1, 2, 3, 4, or 5) amino acid substitutions; (2) a second polypeptide comprising from the N-terminus to the C terminus: an anti-LAG-3 V_{H}H, a peptide linker and a light chain of an anti-PD-1 antibody, and the second polypeptide comprising the amino acid sequence of SEQ ID NO:15, or a variant thereof comprising up to about 5 (such as about any of 1, 2, 3, 4, or 5) amino acid substitutions. In some embodiments, a trispecific antigen binding protein (TABP) comprises: (1) a first polypeptide comprising from the N-terminus to the C terminus: an anti-TIGIT V_{H}H, a peptide linker and a heavy chain of an anti-PD-1 antibody, and the first polypeptide comprising the amino acid sequence of SEQ ID NO:16; (2) a second polypeptide comprising from the N-terminus to the C terminus: an anti-LAG-3 V_{H}H, a peptide linker and a light chain of an anti-PD-1 antibody, and the second polypeptide comprising the amino acid sequence of SEQ ID NO:15 (hereinafter denoted as “TPTL-11”).

In some embodiments, a trispecific antigen binding protein (TABP) comprises: (1) a first polypeptide comprising from the N-terminus to the C-terminus: an anti-TIGIT V_{H}H comprising the amino acid sequence of SEQ ID NO: 31, or a variant thereof comprising up to about 3 (such as about any of 1, 2, or 3) amino acid substitutions, a peptide linker comprising the amino acid sequence of any one of SEQ ID NOs:1-7, an anti-LAG-3 V_{H}H comprising the amino acid sequence of SEQ ID NO: 32, or a variant thereof comprising up to about 3 (such as about any of 1, 2, or 3) amino acid substitutions, a peptide linker comprising the amino acid sequence of any one of SEQ ID NOs:1-7, and a heavy chain of an anti-PD-1 antibody comprising the amino acid sequence of SEQ ID NO:8; and (2) a second polypeptide comprising from the N-terminus to the C-terminus: a light chain of an anti-PD-1 antibody comprising the amino acid sequence of SEQ ID NO:9. In some embodiments, a trispecific antigen binding protein (TABP) comprises: (1) a first polypeptide comprising from the N-terminus to the C-terminus: an anti-TIGIT V_{H}H comprising the amino acid sequence of SEQ ID NO: 31, a peptide linker comprising the amino acid sequence of any one of SEQ ID NOs: 1-3, or 7, an anti-LAG-3 V_{H}H comprising the amino acid sequence of SEQ ID NO: 32, a peptide linker comprising the amino acid sequence of any one of SEQ ID NOs: 1-3, or 7, and a heavy chain of an anti-PD-1 antibody comprising the amino acid sequence of SEQ ID NO:8; and (2) a second polypeptide comprising from the N-terminus to the C-terminus: a light chain of an anti-PD-1 antibody comprising the amino acid sequence of SEQ ID NO:9. In some embodiments, a trispecific antigen binding protein (TABP) comprises: (1) a first polypeptide comprising from the N-terminus to the C terminus: an anti-TIGIT V_{H}H, a peptide linker, an anti-LAG-3 V_{H}H, a peptide linker and a heavy chain of an anti-PD-1 antibody, and the first polypeptide comprising the amino acid sequence of SEQ ID NO:18, or a variant thereof comprising up to about 5 (such as about any of 1, 2, 3, 4, or 5) amino acid substitutions; (2) a second polypeptide comprising from the N-terminus to the C terminus: a light chain of an anti-PD-1 antibody, and the second polypeptide comprising the amino acid sequence of SEQ ID NO:17, or a variant thereof comprising up to about 5 (such as about any of 1, 2, 3, 4, or 5) amino acid substitutions. In some embodiments, a trispecific antigen binding protein (TABP) comprises: (1) a first polypeptide comprising from the N-terminus to the C terminus: an anti-TIGIT V_{H}H, a peptide linker, an anti-LAG-3 V_{H}H, a peptide linker and a heavy chain of an anti-PD-1 antibody, and the first polypeptide comprising the amino acid sequence of SEQ ID NO:18; (2) a second polypeptide comprising from the N-terminus to the C terminus: a light chain of an anti-PD-1 antibody, and the second polypeptide comprising the amino acid sequence of SEQ ID NO:17 (hereinafter denoted as “TPTL-12”).

In some embodiments, a trispecific antigen binding protein (TABP) comprises: (1) a first polypeptide comprising from the N-terminus to the C-terminus: a heavy chain of an anti-PD-1 antibody comprising the amino acid sequence of SEQ ID NO:8; and (2) a second polypeptide comprising from the N-terminus to the C-terminus: an anti-TIGIT V_{H}H comprising the amino acid sequence of SEQ ID NO: 31, or a variant thereof comprising up to about 3 (such as about any of 1, 2, or 3) amino acid substitutions, a peptide linker comprising the amino acid sequence of any one of SEQ ID NOs:1-7, an anti-LAG-3 V_{H}H comprising the amino acid sequence of SEQ ID NO: 32, or a variant thereof comprising up to about 3 (such as about any of 1, 2, or 3) amino acid substitutions, a peptide linker comprising the amino acid sequence of any one of SEQ ID NOs:1-7, and a light chain of an anti-PD-1 antibody comprising the amino acid sequence of SEQ ID NO:9. In some embodiments, a trispecific antigen binding protein (TABP) comprises: (1) a first polypeptide comprising from the N-terminus to the C-terminus: a heavy chain of an anti-PD-1 antibody comprising the amino acid sequence of SEQ ID NO:8; and (2) a second polypeptide comprising from the N-terminus to the C-terminus: an anti-TIGIT V_{H}H comprising the amino acid sequence of SEQ ID NO: 31, a peptide linker comprising the amino acid sequence of any one of SEQ ID NOs:1-3, or 7, an anti-LAG-3 V_{H}H comprising the amino acid sequence of SEQ ID NO: 32, a peptide linker comprising the amino acid sequence of any one of SEQ ID NOs: 1-3, or 7, and a light chain of an anti-PD-1 antibody comprising the amino acid sequence of SEQ ID NO:9. In some embodiments, a trispecific antigen binding protein (TABP) comprises: (1) a first polypeptide comprising from the N-terminus to the C terminus: a heavy chain of an anti-PD-1 antibody, and the first polypeptide comprising the amino acid sequence of SEQ ID NO:20, or a variant thereof comprising up to about 5 (such as about any of 1, 2, 3, 4, or 5) amino acid substitutions; (2) a second polypeptide comprising from the N-terminus to the C terminus: an anti-TIGIT V_{H}H, a peptide linker, an anti-LAG-3 V_{H}H, a peptide linker and a light chain of an anti-PD-1 antibody, and the second polypeptide comprising the amino acid sequence of SEQ ID NO:19, or a variant thereof comprising up to about 5 (such as about any of 1, 2, 3, 4, or 5) amino acid substitutions. In some embodiments, a trispecific antigen binding protein (TABP) comprises: (1) a first polypeptide comprising from the N-terminus to the C terminus: a heavy chain of an anti-PD-1 antibody, and the first polypeptide comprising the amino acid sequence of SEQ ID NO:20; (2) a second polypeptide comprising from the N-terminus to the C terminus: an anti-TIGIT V_{H}H, a peptide linker, an anti-LAG-3 V_{H}H, a peptide linker and a light chain of an anti-PD-1 antibody, and the second polypeptide comprising the amino acid sequence of SEQ ID NO:19 (hereinafter denoted as “TPTL-13”).

In some embodiments, a trispecific antigen binding protein (TABP) comprises: (1) a first polypeptide comprising from the N-terminus to the C-terminus: a heavy chain of an anti-PD-1 antibody comprising the amino acid sequence of SEQ ID NO:8; and (2) a second polypeptide comprising from the N-terminus to the C-terminus: an anti-TIGIT V_{H}H comprising the amino acid sequence of SEQ ID NO: 31, or a variant thereof comprising up to about 3 (such as about any of 1, 2, or 3) amino acid substitutions, a peptide linker comprising the amino acid sequence of any one of SEQ ID NOs:1-7, a light chain of an anti-PD-1 antibody comprising the amino acid sequence of SEQ ID NO:9, a peptide linker comprising the amino acid sequence of any one of SEQ ID NOs:1-7, and an anti-LAG-3 V_{H}H comprising the amino acid sequence of SEQ ID NO: 32, or a variant thereof comprising up to about 3 (such as about any of 1, 2, or 3) amino acid substitutions. In some embodiments, a trispecific antigen binding protein (TABP) comprises: (1) a first polypeptide comprising from the N-terminus to the C-terminus: a heavy chain of an anti-PD-1 antibody comprising the amino acid sequence of SEQ ID NO:8; and (2) a second polypeptide comprising from the N-terminus to the C-terminus: an anti-TIGIT V_{H}H comprising the amino acid sequence of SEQ ID NO: 31, a peptide linker comprising the amino acid sequence of any one of SEQ ID NOs:1-3, or 7, a light chain of an anti-PD-1 antibody comprising the amino acid sequence of SEQ ID NO:9, a peptide linker comprising the amino acid sequence of any one of SEQ ID NOs:1-3, or 7, and an anti-LAG-3 V_{H}H comprising the amino acid sequence of SEQ ID NO: 32. In some embodiments, a trispecific antigen binding protein (TABP) comprises: (1) a first polypeptide comprising from the N-terminus to the C terminus: a heavy chain of an anti-PD-1 antibody, and the first polypeptide comprising the amino acid sequence of SEQ ID NO:20, or a variant thereof comprising up to about 5 (such as about any of 1, 2, 3, 4, or 5) amino acid substitutions; (2) a second polypeptide comprising from the N-terminus to the C terminus: an anti-TIGIT V_{H}H, a peptide linker, a light chain of an anti-PD-1 antibody, a peptide linker and an anti-LAG-3 V_{H}H, and the second polypeptide comprising the amino acid sequence of SEQ ID NO:21, or a variant thereof comprising up to about 5 (such as about any of 1, 2, 3, 4, or 5) amino acid substitutions. In some embodiments, a trispecific antigen binding protein (TABP) comprises: (1) a first polypeptide comprising from the N-terminus to the C terminus: a heavy chain of an anti-PD-1 antibody, and the first polypeptide comprising the amino acid sequence of SEQ ID NO:20; (2) a second polypeptide comprising from the N-terminus to the C terminus: an anti-TIGIT V_{H}H, a peptide linker, a light chain of an anti-PD-1 antibody, a peptide linker and an anti-LAG-3 V_{H}H, and the second polypeptide comprising the amino acid sequence of SEQ ID NO:21 (hereinafter denoted as “TPTL-14”).

In some embodiments, a trispecific antigen binding protein (TABP) comprises: (1) a first polypeptide comprising from the N-terminus to the C-terminus: an anti-TIGIT V_{H}H comprising the amino acid sequence of SEQ ID NO: 31, or a variant thereof comprising up to about 3 (such as about any of 1, 2, or 3) amino acid substitutions, a peptide linker comprising the amino acid sequence of any one of SEQ ID NOs:1-7, and a heavy chain of an anti-PD-1 antibody comprising the amino acid sequence of SEQ ID NO:8; and (2) a second polypeptide comprising from the N-terminus to the C-terminus: a light chain of an anti-PD-1 antibody comprising the amino acid sequence of SEQ ID NO:9, a peptide linker comprising the amino acid sequence of any one of SEQ ID NOs:1-7, and an anti-LAG-3 V_{H}H comprising the amino acid sequence of SEQ ID NO: 32, or a variant thereof comprising up to about 3 (such as about any of 1, 2, or 3) amino acid substitutions. In some embodiments, a trispecific antigen binding protein (TABP) comprises: (1) a first polypeptide comprising from the N-terminus to the C-terminus: an anti-TIGIT V_{H}H comprising the amino acid sequence of SEQ ID NO: 31, a peptide linker comprising the amino acid sequence of any one of SEQ ID NOs:1-3, or 7, and a heavy chain of an anti-PD-1 antibody comprising the amino acid sequence of SEQ ID NO:8; and (2) a second polypeptide comprising from the N-terminus to the C-terminus: a light chain of an anti-PD-1 antibody comprising the amino acid sequence of SEQ ID NO:9, a peptide linker comprising the amino acid sequence of any one of SEQ ID NOs:1-3, or 7, and an anti-LAG-3 V_{H}H comprising the amino acid sequence of SEQ ID NO: 32. In some embodiments, a trispecific antigen binding protein (TABP) comprises: (1) a first polypeptide comprising from the N-terminus to the C terminus: an anti-TIGIT V_{H}H, a peptide linker and a heavy chain of an anti-PD-1 antibody, and the first polypeptide comprising the amino acid sequence of SEQ ID NO:16, or a variant thereof comprising up to about 5 (such as about any of 1, 2, 3, 4, or 5) amino acid substitutions; (2) a second polypeptide comprising from the N-terminus to the C terminus: a light chain of an anti-PD-1 antibody, a peptide linker and an anti-LAG-3 V_{H}H, and the second polypeptide comprising the amino acid sequence of SEQ ID NO:22, or a variant thereof comprising up to about 5 (such as about any of 1, 2, 3, 4, or 5) amino acid substitutions. In some embodiments, a trispecific antigen binding protein (TABP) comprises: (1) a first polypeptide comprising from the N-terminus to the C terminus: an anti-TIGIT V_{H}H, a peptide linker and a heavy chain of an anti-PD-1 antibody, and the first polypeptide comprising the amino acid sequence of SEQ ID NO:16; (2) a second polypeptide comprising from the N-terminus to the C terminus: a light chain of an anti-PD-1 antibody, a peptide linker and an anti-LAG-3 V_{H}H, and the second polypeptide comprising the amino acid sequence of SEQ ID NO:22 (hereinafter denoted as “TPTL-15”).

In some embodiments, a trispecific antigen binding protein (TABP) comprises: (1) a first polypeptide comprising from the N-terminus to the C-terminus: an anti-TIGIT V_{H}H comprising the amino acid sequence of SEQ ID NO: 31, or a variant thereof comprising up to about 3 (such as about any of 1, 2, or 3) amino acid substitutions, a peptide linker comprising the amino acid sequence of any one of SEQ ID NOs:1-7, a heavy chain of an anti-PD-1 antibody comprising the amino acid sequence of SEQ ID NO:8, a peptide linker comprising the amino acid sequence of any one of SEQ ID NOs:1-7, an anti-LAG-3 V_{H}H comprising the amino acid sequence of SEQ ID NO: 32, or a variant thereof comprising up to about 3 (such as about any of 1, 2, or 3) amino acid substitutions; and (2) a second polypeptide comprising from the N-terminus to the C-terminus: a light chain of an anti-PD-1 antibody comprising the amino acid sequence of SEQ ID NO:9. In some embodiments, a trispecific antigen binding protein (TABP) comprises: (1) a first polypeptide comprising from the N-terminus to the C-terminus: an anti-TIGIT V_{H}H comprising the amino acid sequence of SEQ ID NO: 31, a peptide linker comprising the amino acid sequence of any one of SEQ ID NOs:1-3, or 7, a heavy chain of an anti-PD-1 antibody comprising the amino acid sequence of SEQ ID NO:8, a peptide linker comprising the amino acid sequence of any one of SEQ ID NOs:1-3, or 7, an anti-LAG-3 V_{H}H comprising the amino acid sequence of SEQ ID NO: 32; and (2) a second polypeptide comprising from the N-terminus to the C-terminus: a light chain of an anti-PD-1 antibody comprising the amino acid sequence of SEQ ID NO:9. In some embodiments, a trispecific antigen binding protein (TABP) comprises: (1) a first polypeptide comprising from the N-terminus to the C terminus: an anti-TIGIT V_{H}H, a peptide linker, a heavy chain of an anti-PD-1 antibody, a peptide linker and an anti-LAG-3 V_{H}H, and the first polypeptide comprising the amino acid sequence of SEQ ID NO:23, or a variant thereof comprising up to about 5 (such as about any of 1, 2, 3, 4, or 5) amino acid substitutions; (2) a second polypeptide comprising from the N-terminus to the C terminus: a light chain of an anti-PD-1 antibody, and the second polypeptide comprising the amino acid sequence of SEQ ID NO:17, or a variant thereof comprising up to about 5 (such as about any of 1, 2, 3, 4, or 5) amino acid substitutions. In some embodiments, a trispecific antigen binding protein (TABP) comprises: (1) a first polypeptide comprising from the N-terminus to the C terminus: an anti-TIGIT V_{H}H, a peptide linker, a heavy chain of an anti-PD-1 antibody, a peptide linker and an anti-LAG-3 V_{H}H, and the first polypeptide comprising the amino acid sequence of SEQ ID NO:23; (2) a second polypeptide comprising from the N-terminus to the C terminus: a light chain of an anti-PD-1 antibody, and the second polypeptide comprising the amino acid sequence of SEQ ID NO:17 (hereinafter denoted as “TPTL-16”).

In some embodiments, a trispecific antigen binding protein (TABP) comprises: (1) a first polypeptide comprising from the N-terminus to the C-terminus: a heavy chain of an anti-PD-1 antibody comprising the amino acid sequence of SEQ ID NO:8, a peptide linker comprising the amino acid sequence of any one of SEQ ID NOs:1-7, and an anti-LAG-3 V_{H}H comprising the amino acid sequence of SEQ ID NO: 32, or a variant thereof comprising up to about 3 (such as about any of 1, 2, or 3) amino acid substitutions; and (2) a second polypeptide comprising from the N-terminus to the C-terminus: an anti-TIGIT V_{H}H comprising the amino acid sequence of SEQ ID NO: 31, or a variant thereof comprising up to about 3 (such as about any of 1, 2, or 3) amino acid substitutions, a peptide linker comprising the amino acid sequence of any one of SEQ ID NOs:1-7, and a light chain of an anti-PD-1 antibody comprising the amino acid sequence of SEQ ID NO:9. In some embodiments, a trispecific antigen binding protein (TABP) comprises: (1) a first polypeptide comprising from the N-terminus to the C-terminus: a heavy chain of an anti-PD-1 antibody comprising the amino acid sequence of SEQ ID NO:8, a peptide linker comprising the amino acid sequence of any one of SEQ ID NOs:1-3, or 7, and an anti-LAG-3 V_{H}H comprising the amino acid sequence of SEQ ID NO: 32; and (2) a second polypeptide comprising from the N-terminus to the C-terminus: an anti-TIGIT V_{H}H comprising the amino acid sequence of SEQ ID NO: 31, a peptide linker comprising the amino acid sequence of any one of SEQ ID NOs:1-3, or 7, and a light chain of an anti-PD-1 antibody comprising the amino acid sequence of SEQ ID NO:9. In some embodiments, a trispecific antigen binding protein (TABP) comprises: (1) a first polypeptide comprising from the N-terminus to the C terminus: a heavy chain of an anti-PD-1 antibody, a peptide linker and an anti-LAG-3 V_{H}H, and the first polypeptide comprising the amino acid sequence of SEQ ID NO:25, or a variant thereof comprising up to about 5 (such as about any of 1, 2, 3, 4, or 5) amino acid substitutions; (2) a second polypeptide comprising from the N-terminus to the C terminus: an anti-TIGIT V_{H}H, a peptide linker and a light chain of an anti-PD-1 antibody, and the second polypeptide comprising the amino acid sequence of SEQ ID NO:24, or a variant thereof comprising up to about 5 (such as about any of 1, 2, 3, 4, or 5) amino acid substitutions. In some embodiments, a trispecific antigen binding protein (TABP) comprises: (1) a first polypeptide comprising from the N-terminus to the C terminus: a heavy chain of an anti-PD-1 antibody, a peptide linker and an anti-LAG-3 V_{H}H, and the first polypeptide comprising the amino acid sequence of SEQ ID NO:25; (2) a second polypeptide comprising from the N-terminus to the C terminus: an anti-TIGIT V_{H}H, a peptide linker and a light chain of an anti-PD-1 antibody, and the second polypeptide comprising the amino acid sequence of SEQ ID NO:24 (hereinafter denoted as “TPTL-17”).

In some embodiments, a trispecific antigen binding protein (TABP) comprises: (1) a first polypeptide comprising from the N-terminus to the C-terminus: a heavy chain of an anti-PD-1 antibody comprising the amino acid sequence of SEQ ID NO:8, a peptide linker comprising the amino acid sequence of any one of SEQ ID NOs:1-7, and an anti-TIGIT V_{H}H comprising the amino acid sequence of SEQ ID NO: 31, or a variant thereof comprising up to about 3 (such as about any of 1, 2, or 3) amino acid substitutions; and (2) a second polypeptide comprising from the N-terminus to the C-terminus: a light chain of an anti-PD-1 antibody comprising the amino acid sequence of SEQ ID NO:9, a peptide linker comprising the amino acid sequence of any one of SEQ ID NOs:1-7, and an anti-LAG-3 V_{H}H comprising the amino acid sequence of SEQ ID NO: 32, or a variant thereof comprising up to about 3 (such as about any of 1, 2, or 3) amino acid substitutions. In some embodiments, a trispecific antigen binding protein (TABP) comprises: (1) a first polypeptide comprising from the N-terminus to the C-terminus: a heavy chain of an anti-PD-1 antibody comprising the amino acid sequence of SEQ ID NO:8, a peptide linker comprising the amino acid sequence of any one of SEQ ID NOs:1-3, or 7, and an anti-TIGIT V_{H}H comprising the amino acid sequence of SEQ ID NO: 31; and (2) a second polypeptide comprising from the N-terminus to the C-terminus: a light chain of an anti-PD-1 antibody comprising the amino acid sequence of SEQ ID NO:9, a peptide linker comprising the amino acid sequence of any one of SEQ ID NOs:1-3, or 7, and an anti-LAG-3 V_{H}H comprising the amino acid sequence of SEQ ID NO: 32. In some embodiments, a trispecific antigen binding protein (TABP) comprises: (1) a first polypeptide comprising from the N-terminus to the C terminus: a heavy chain of an anti-PD-1 antibody, a peptide linker and an anti-TIGIT V_{H}H, and the first polypeptide comprising the amino acid sequence of SEQ ID NO:26, or a variant thereof comprising up to about 5 (such as about any of 1, 2, 3, 4, or 5) amino acid substitutions; (2) a second polypeptide comprising from the N-terminus to the C terminus: a light chain of an anti-PD-1 antibody, a peptide linker and an anti-LAG-3 V_{H}H, and the second polypeptide comprising the amino acid sequence of SEQ ID NO:22, or a variant thereof comprising up to about 5 (such as about any of 1, 2, 3, 4, or 5) amino acid substitutions. In some embodiments, a trispecific antigen binding protein (TABP) comprises: (1) a first polypeptide comprising from the N-terminus to the C terminus: a heavy chain of an anti-PD-1 antibody, a peptide linker and an anti-TIGIT V_{H}H, and the first polypeptide comprising the amino acid sequence of SEQ ID NO:26; (2) a second polypeptide comprising from the N-terminus to the C terminus: a light chain of an anti-PD-1 antibody, a peptide linker and an anti-LAG-3 V_{H}H, and the second polypeptide comprising the amino acid sequence of SEQ ID NO:22 (hereinafter denoted as “TPTL-18”).

In some embodiments, a trispecific antigen binding protein (TABP) comprises: (1) a first polypeptide comprising from the N-terminus to the C-terminus: a heavy chain of an anti-PD-1 antibody comprising the amino acid sequence of SEQ ID NO:8; and (2) a second polypeptide comprising from the N-terminus to the C-terminus: a light chain of an anti-PD-1 antibody comprising the amino acid sequence of SEQ ID NO:9, a peptide linker comprising the amino acid sequence of any one of SEQ ID NOs:1-7, an anti-LAG-3 V_{H}H comprising the amino acid sequence of SEQ ID NO: 32, or a variant thereof comprising up to about 3 (such as about any of 1, 2, or 3) amino acid substitutions, a peptide linker comprising the amino acid sequence of any one of SEQ ID NOs:1-7, and an anti-TIGIT V_{H}H comprising the amino acid sequence of SEQ ID NO: 31, or a variant thereof comprising up to about 3 (such as about any of 1, 2, or 3) amino acid substitutions. In some embodiments, a trispecific antigen binding protein (TABP) comprises: (1) a first polypeptide comprising from the N-terminus to the C-terminus: a heavy chain of an anti-PD-1 antibody comprising the amino acid sequence of SEQ ID NO:8; and (2) a second polypeptide comprising from the N-terminus to the C-terminus: a light chain of an anti-PD-1 antibody comprising the amino acid sequence of SEQ ID NO:9, a peptide linker comprising the amino acid sequence of any one of SEQ ID NOs:1-3, or 7, an anti-LAG-3 V_{H}H comprising the amino acid sequence of SEQ ID NO: 32, a peptide linker comprising the amino acid sequence of any one of SEQ ID NOs:1-3, or 7, and an anti-TIGIT V_{H}H comprising the amino acid sequence of SEQ ID NO: 31. In some embodiments, a trispecific antigen binding protein (TABP) comprises: (1) a first polypeptide comprising from the N-terminus to the C terminus: a heavy chain of an anti-PD-1 antibody, and the first polypeptide comprising the amino acid sequence of SEQ ID NO:20, or a variant thereof comprising up to about 5 (such as about any of 1, 2, 3, 4, or 5) amino acid substitutions; (2) a second polypeptide comprising from the N-terminus to the C terminus: a light chain of an anti-PD-1 antibody, a peptide linker, an anti-LAG-3 V_{H}H, a peptide linker and an anti-TIGIT V_{H}H, and the second polypeptide comprising the amino acid sequence of SEQ ID NO:27, or a variant thereof comprising up to about 5 (such as about any of 1, 2, 3, 4, or 5) amino acid substitutions. In some embodiments, a trispecific antigen binding protein (TABP) comprises: (1) a first polypeptide comprising from the N-terminus to the C terminus: a heavy chain of an anti-PD-1 antibody, and the first polypeptide comprising the amino acid sequence of SEQ ID NO:20; (2) a second polypeptide comprising from the N-terminus to the C terminus: a light chain of an anti-PD-1 antibody, a peptide linker, an anti-LAG-3 V_{H}H, a peptide linker and an anti-TIGIT V_{H}H, and the second polypeptide comprising the amino acid sequence of SEQ ID NO:27 (hereinafter denoted as “TPTL-19”).

In some embodiments, a trispecific antigen binding protein (TABP) comprises: (1) a first polypeptide comprising from the N-terminus to the C-terminus: a heavy chain of an anti-PD-1 antibody comprising the amino acid sequence of SEQ ID NO:8, a peptide linker comprising the amino acid sequence of any one of SEQ ID NOs:1-7, an anti-LAG-3 V_{H}H comprising the amino acid sequence of SEQ ID NO: 32, or a variant thereof comprising up to about 3 (such as about any of 1, 2, or 3) amino acid substitutions, a peptide linker comprising the amino acid sequence of any one of SEQ ID NOs:1-7, and an anti-TIGIT V_{H}H comprising the amino acid sequence of SEQ ID NO: 31, or a variant thereof comprising up to about 3 (such as about any of 1, 2, or 3) amino acid substitutions; and (2) a second polypeptide comprising from the N-terminus to the C-terminus: a light chain of an anti-PD-1 antibody comprising the amino acid sequence of SEQ ID NO:9. In some embodiments, a trispecific antigen binding protein (TABP) comprises: (1) a first polypeptide comprising from the N-terminus to the C-terminus: a heavy chain of an anti-PD-1 antibody comprising the amino acid sequence of SEQ ID NO:8, a peptide linker comprising the amino acid sequence of any one of SEQ ID NOs:1-3, or 7, an anti-LAG-3 V_{H}H comprising the amino acid sequence of SEQ ID NO: 32, a peptide linker comprising the amino acid sequence of any one of SEQ ID NOs:1-3, or 7, and an anti-TIGIT V_{H}H comprising the amino acid sequence of SEQ ID NO: 31; and (2) a second polypeptide comprising from the N-terminus to the C-terminus: a light chain of an anti-PD-1 antibody comprising the amino acid sequence of SEQ ID NO:9. In some embodiments, a trispecific antigen binding protein (TABP) comprises: (1) a first polypeptide comprising from the N-terminus to the C terminus: a heavy chain of an anti-PD-1 antibody, a peptide linker, an anti-LAG-3 V_{H}H, a peptide linker and an anti-TIGIT V_{H}H, and the first polypeptide comprising the amino acid sequence of SEQ ID NO:28, or a variant thereof comprising up to about 5 (such as about any of 1, 2, 3, 4, or 5) amino acid substitutions; (2) a second polypeptide comprising from the N-terminus to the C terminus: a light chain of an anti-PD-1 antibody, and the second polypeptide comprising the amino acid sequence of SEQ ID NO:17, or a variant thereof comprising up to about 5 (such as about any of 1, 2, 3, 4, or 5) amino acid substitutions. In some embodiments, a trispecific antigen binding protein (TABP) comprises: (1) a first polypeptide comprising from the N-terminus to the C terminus: a heavy chain of an anti-PD-1 antibody, a peptide linker, an anti-LAG-3 V_{H}H, a peptide linker and an anti-TIGIT V_{H}H, and the first polypeptide comprising the amino acid sequence of SEQ ID NO:28; (2) a second polypeptide comprising from the N-terminus to the C terminus: a light chain of an anti-PD-1 antibody, and the second polypeptide comprising the amino acid sequence of SEQ ID NO:17 (hereinafter denoted as “TPTL-20”).

### Antigen Binding Portion Comprising Single-Domain Antibody

The MABPs of the present application comprise at least two antigen binding portions each comprising an sdAb. Exemplary sdAbs include, but are not limited to, heavy chain variable domains from heavy-chain only antibodies (e.g., V_{H}H or V_{NM}), binding molecules naturally devoid of light chains, single domains (such as V_{H} or V_{L}) derived from conventional 4-chain antibodies, humanized heavy-chain only antibodies, human sdAbs produced by transgenic mice or rats expressing human heavy chain segments, and engineered domains and single domain scaffolds other than those derived from antibodies. Any sdAbs known in the art or developed by the inventors may be used to construct the MABPs of the present application. The sdAbs may be derived from any species including, but not limited to mouse, rat, human, camel, llama, lamprey, fish, shark, goat, rabbit, and bovine. Single-domain antibodies contemplated herein also include naturally occurring sdAb molecules from species other than Camelidae and sharks.

In some embodiments, the first sdAb and/or the second sdAb is derived from a naturally occurring single-domain antigen binding molecule known as heavy chain antibody devoid of light chains (also referred herein as “heavy chain only antibodies”). Such single domain molecules are disclosed in WO 94/04678 and Hamers-Casterman, C. et al. (1993) Nature 363:446-448, for example. For clarity reasons, the variable domain derived from a heavy chain molecule naturally devoid of light chain is known herein as a V_{H}H to distinguish it from the conventional VH of four chain immunoglobulins. Such a V_{H}H molecule can be derived from antibodies raised in Camelidae species, for example, camel, llama, vicuna, dromedary, alpaca and guanaco. Other species besides Camelidae may produce heavy chain molecules naturally devoid of light chain, and such V_{H}Hs are within the scope of the present application.

V_{H}H molecules from Camelids are about 10 times smaller than IgG molecules. They are single polypeptides and can be very stable, resisting extreme pH and temperature conditions. Moreover, they can be resistant to the action of proteases which is not the case for conventional antibodies. Furthermore, in vitro expression of V_{H}Hs produces high yield, properly folded functional V_{H}Hs. In addition, antibodies generated in Camelids can recognize epitopes other than those recognized by antibodies generated in vitro through the use of antibody libraries or via immunization of mammals other than Camelids (see, for example, WO9749805). As such, MABPs comprising V_{H}H domains may interact more efficiently with targets than conventional antibodies. Since V_{H}Hs are known to bind into ‘unusual’ epitopes such as cavities or grooves, the affinity of MABPs comprising such V_{H}Hs may be more suitable for therapeutic treatment than conventional multispecific polypeptides.

In some embodiments, there is provided a trispecific antigen binding protein comprising: (a) a first antigen binding portion comprising a heavy chain variable domain (V_{H}) and a light chain variable domain (V_{L}), wherein the V_{H} and V_{L} together form an antigen-binding site that specifically binds a first epitope, (b) a second antigen binding portion comprising a first V_{H}H domain that specifically binds a second epitope, and (c) a third antigen binding portion comprising a second V_{H}H domain that specifically binds a third epitope, wherein the first antigen binding portion, the second antigen binding portion, and the third antigen binding portion are fused to each other. In some embodiments, the first antigen binding portion comprises a heavy chain comprising the V_{H} and a light chain comprising the V_{L}. In some embodiments, the first antigen binding region is a full-length antibody consisting of two heavy chains and two light chains. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG1 Fc or IgG4 Fc.

In some embodiments, the first sdAb and/or the second sdAb is derived from a variable region of the immunoglobulin found in cartilaginous fish. For example, the sdAb can be derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

In some embodiments, the first sdAb and/or the second sdAb is recombinant, CDR-grafted, humanized, camelized, de-immunized and/or in vitro generated (e.g., selected by phage display). In some embodiments, the first sdAb and/or the second sdAb is a human sdAb produced by transgenic mice or rats expressing human heavy chain segments. See, e.g., US20090307787A1, U.S. Pat. No. 8,754,287, US20150289489A1, US20100122358A1 and WO2004049794. In some embodiments, the first sdAb and/or the second sdAb is affinity matured.

An sdAb comprising a V_{H}H domain can be humanized to have human-like sequences. In some embodiments, the FR regions of the V_{H}H domain used herein comprise at least about any one of 50%, 60%, 70%, 80%, 90%, 95% or more of amino acid sequence homology to human VH framework regions. One exemplary class of humanized V_{H}H domains is characterized in that the V_{H}Hs carry an amino acid from the group consisting of glycine, alanine, valine, leucine, isoleucine, proline, phenylalanine, tyrosine, tryptophan, methionine, serine, threonine, asparagine, or glutamine at position 45, such as, for example, L45 and a tryptophan at position 103, according to the Kabat numbering. As such, polypeptides belonging to this class show a high amino acid sequence homology to human VH framework regions and said polypeptides might be administered to a human directly without expectation of an unwanted immune response therefrom, and without the burden of further humanization.

Another exemplary class of humanized Camelidae sdAbs has been described in WO 03/035694 and contains hydrophobic FR2 residues typically found in conventional antibodies of human origin or from other species, but compensating this loss in hydrophilicity by the charged arginine residue on position 103 that substitutes the conserved tryptophan residue present in V_{H} from double-chain antibodies. As such, peptides belonging to these two classes show a high amino acid sequence homology to human V_{H} framework regions and said peptides might be administered to a human directly without expectation of an unwanted immune response therefrom, and without the burden of further humanization.

In some embodiments, the MABP comprises naturally produced sdAbs or derivatives thereof, such as a Camelid sdAb, or a humanized sdAb derived from a Camelid sdAb. In some embodiments, the first sdAb and/or the second sdAb is obtained from llama. In some embodiments, the first sdAb and/or the second sdAb is further engineered to remove sequences not normally found in human antibodies (such as CDR regions or CDR-FR junctions).

The first sdAb and the second sdAb of the MABP have suitable affinities to their epitopes. For example, the affinity of each sdAb may affect the overall affinity and avidity of the MABP to the target cell or tissue, which may further affect the efficacy of the MABP. In some embodiments, the first sdAb and/or the second sdAb binds its epitope with high affinity. A high-affinity sdAb binds its epitope with a dissociation constant (K_{d}) in the low nanomolar (10⁻⁹ M) range, such as no more than about any of 5 nM, 4 nM, 3 nM, 2 nM, 1 nM, 0.5 nM, 0.2 nM, 0.1 nM, 0.05 nM, 0.02 nM, 0.01 nM, 5 pM, 2 pM, 1 pM or less. In some embodiments, the first sdAb and/or the second sdAb binds its epitope with low affinity. A low-affinity sdAb binds its epitope with a K_{d} in the low micromolar (10⁻⁶ M) range or higher, such as more than about any of 1 μM, 2 μM, 3 μM, 4 μM, 5 μM, 6 μM, 7 μM, 8 μM, 9 μM, 10 μM or more. In some embodiments, the first sdAb and/or the second sdAb binds its epitope with medium affinity. A medium-affinity sdAb binds its epitope with a K_{d} lower than that of a low-affinity sdAb but higher than that of a high-affinity sdAb. In some embodiments, a medium-affinity sdAb binds its epitope with a K_{d} of any one of about 1 nM to about 10 nM, about 10 nM to about 100 nM, about 100 nM to about 500 nM, about 500 nM to about 1 μM, about 1 nM to about 100 nM, about 10 nM to about 500 nM, or about 1 nM to about 1 μM.

In some embodiments, the first sdAb and/or the second sdAb has a stronger affinity to its epitope than the antigen binding portion comprising V_{H} and V_{L}. In some embodiments, the first sdAb and/or the second sdAb has a weaker affinity to its epitope than the antigen binding portion comprising V_{H} and V_{L}. In some embodiments, the difference between the affinity between the first sdAb and/or the second sdAb to its epitope and the antigen binding portion comprising V_{H} and V_{L} and its epitope is about at least any of 2×, 5×, 10×, 100×, 1000× or more. In some embodiments, the affinity between the first sdAb and/or the second sdAb to its epitope is comparable to that between the antigen binding portion comprising V_{H} and V_{L} and its epitope.

In some embodiments, the first sdAb and/or the second sdAb specifically binds an immune checkpoint molecule. In some embodiments, the first sdAb and/or the second sdAb specifically binds a stimulatory immune checkpoint molecule. In some embodiments, the first sdAb and/or the second sdAb specifically binds an inhibitory immune checkpoint molecule. In some embodiments, the first sdAb and/or the second sdAb specifically binds an immune checkpoint molecule selected from the group consisting of PD-1, PD-L1, PD-L2, CTLA-4, B7-H3, TIM-3, LAG-3, TIGIT, VISTA, ICOS, 4-1BB, OX40, GITR, and CD40. In some embodiments, the first sdAb and/or the second sdAb is an agonist for the immune checkpoint molecule. In some embodiments, the first sdAb and/or the second sdAb is an antagonist against the immune checkpoint molecule.

In some embodiments, the first sdAb specifically binds TIGIT. In some embodiments, the first sdAb binds TIGIT with high affinity. In some embodiments, the first sdAb binds TIGIT with medium affinity. In some embodiments, the first sdAb binds TIGIT with low affinity. In some embodiments, the second sdAb specifically binds LAG-3. In some embodiments, the second sdAb binds LAG-3 with high affinity. In some embodiments, the second sdAb binds LAG-3 with medium affinity. In some embodiments, the second sdAb binds LAG-3 with low affinity.

In some embodiments, the first sdAb specifically binds TIGIT comprising the amino acid sequence of SEQ ID NO: 31, or a variant thereof comprising up to about 3 (such as about any of 1, 2, or 3) amino acid substitutions. In some embodiments, the first sdAb specifically binds TIGIT comprising the amino acid sequence of SEQ ID NO: 31. In some embodiments, the second sdAb specifically binds LAG-3 comprising the amino acid sequence of SEQ ID NO: 32, or a variant thereof comprising up to about 3 (such as about any of 1, 2, or 3) amino acid substitutions. In some embodiments, the second sdAb specifically binds LAG-3 comprising the amino acid sequence of SEQ ID NO: 32.

In some embodiments, the first sdAb and/or the second sdAb specifically binds a cell surface antigen. In some embodiments, the cell surface antigen is a tumor antigen. In some embodiments, the tumor antigen is selected from the group consisting of HER2, BRAF, EGFR, VEGFR2, CD20, RANKL, CD38, and CD52. In some embodiments, the first sdAb and/or the second sdAb specifically binds a cell surface antigen on an immune effector cell, such as T cell, or Natural Killer cell.

In some embodiments, the first sdAb specifically binds CD3. In some embodiments, the first sdAb binds CD3 with high affinity. In some embodiments, the first sdAb binds CD3 with medium affinity. In some embodiments, the first sdAb binds CD3 with low affinity. In some embodiments, the second sdAb binds EGFR. In some embodiments, the second sdAb binds EGFR antigen with high affinity. In some embodiments, the second sdAb binds EGFR with medium affinity. In some embodiments, the second sdAb binds EGFR with low affinity.

In some embodiments, the first sdAb and/or the second sdAb specifically binds an extracellular protein, such as a secreted protein. In some embodiments, the first sdAb and/or the second sdAb specifically binds a pro-inflammatory molecule, such as TNF-α, IL-17A, IL-17F, IL-1β, TNF-α, IL-5, IL-6, IL-6R, or eotaxin-1.

In some embodiments, the first sdAb and/or the second sdAb specifically binds an angiogenic factor, such as VEGF, Ang2, or DLL4.

Antigen Binding Portion Comprising V_{H} and V_{L}

The MABPs of the present application comprise at least one antigen binding portion comprising a heavy chain variable domain (V_{H}) and a light chain variable domain (V_{L}). Such antigen binding portion can be a full-length conventional antibody consisting of two heavy chains and two light chains, or an antigen binding fragment derived therefrom.

In some embodiments, the first antigen binding portion is an antigen binding fragment comprising a heavy chain comprising the V_{H} domain and a light chain comprising the V_{L} domain. Exemplary antigen binding fragments contemplated herein include, but are not limited to, Fab, Fab′, F(ab′)₂, and Fv fragments; diabodies; linear antibodies; single-chain antibody molecules (such as scFv); and multispecific antibodies formed from antibody fragments.

In some embodiments, the first antigen binding portion comprises an Fc region, such as a human Fc region. In some embodiments, the Fc region is derived from an IgG molecule, such as any one of the IgG1, IgG2, IgG3, or IgG4 subclass. In some embodiments, the Fc region is capable of mediating an antibody effector function, such as ADCC (antibody-dependent cell-mediated cytotoxicity) and/or CDC (complement-dependent cytotoxicity). For example, antibodies of subclass IgG1, IgG2, and IgG3 with wildtype Fc sequences usually show complement activation including CIq and C3 binding, whereas IgG4 does not activate the complement system and does not bind CIq and/or C3. In some embodiments, the Fc region comprises a modification that reduces binding affinity of the Fc region to an Fc receptor. In some embodiments, the Fc region is an IgG1 Fc. In some embodiments, the IgG1 Fc comprises one or mutations in positions 233-236, such as L234A and/or L235A. In some embodiments, the Fc region is an IgG4 Fc. In some embodiments, the IgG4 Fc comprises a mutation in positions 327, 330 and/or 331. See, for example, Armour K L et al., Eur f. Immunol. 1999; 29: 2613; and Shields R L et al., J. Biol. Chem. 2001; 276: 6591. In some embodiments, the Fc region comprises a P329G mutation.

In some embodiments, the Fc region comprises a modification that promotes heterodimerization of two non-identical heavy chains. Such modified Fc regions may be of particular interest for MABPs described herein having an asymmetric design. In some embodiments, said modification is a knob-into-hole modification, comprising a knob modification in one of the heavy chains or heavy chain fusion polypeptides and a hole modification in the other one of the two heavy chains or heavy chain fusion polypeptides. In one embodiment, the Fc region comprises a modification within the interface between the two heavy chains in the CH3 domain, wherein i) in the CH3 domain of one heavy chain, an amino acid residue is replaced with an amino acid residue having a larger side chain volume, thereby generating a protuberance (“knob”) within the interface in the CH3 domain of one heavy chain which is positionable in a cavity (“hole”) within the interface in the CH3 domain of the other heavy chain, and ii) in the CH3 domain of the other heavy chain, an amino acid residue is replaced with an amino acid residue having a smaller side chain volume, thereby generating a cavity (“hole”) within the interface in the second CH3 domain within which a protuberance (“knob”) within the interface in the first CH3 domain is positionable. Examples of knob-into-hole modifications have been described, for example, in US 2011/0287009, US2007/0178552, WO 96/027011, WO 98/050431, and Zhu et al., 1997, Protein Science 6:781-788. Other modifications to the Fc region that promote heterodimerization are also contemplated herein. For example, electrostatic steering effects can be engineered into the Fc region to provide Fc-heterodimeric molecules (see, e.g., U.S. Pat. No. 4,676,980, and Brennan et al., Science, 229: 81 (1985)).

In some embodiments, the Fc region comprises a modification that inhibits Fab arm exchange. For example, the S228P mutation in IgG4 Fc prevents Fab arm exchange.

In some embodiments, the first antigen binding portion comprises a kappa light chain constant region. In some embodiments, the first antigen binding portion comprises a lambda light chain constant region. In some embodiments, the first antigen binding portion comprises a light chain constant region comprising the amino acid sequence of SEQ ID NO: 9. In some embodiments, the first antigen binding portion comprises a heavy chain constant region comprising the amino acid sequence of SEQ ID NO: 8.

In some embodiments, the first antigen binding portion is a full-length antibody consisting of two heavy chains and two light chains. In some embodiments, the first antigen binding portion comprises a monoclonal antibody consisting of two heavy chains and two light chains (also referred herein as “4-chain antibody”). In some embodiments, the first antigen binding portion comprises a multispecific (such as trispecific) full-length antibody consisting of two heavy chains and two light chains. In some embodiments, the first antigen binding portion comprises a full-length antibody of human IgG1 subclass, or of human IgG1 subclass with the mutations L234A and L235A. In some embodiments, the first antigen binding portion comprises a full-length antibody of human IgG2 subclass. In some embodiments, the first antigen binding portion comprises a full-length antibody of human IgG3 subclass. In some embodiments, the first antigen binding portion comprises a full-length antibody of human IgG4 subclass or, of human IgG4 subclass with the additional mutation S228P.

Any full-length 4-chain antibody known in the art or antigen binding fragments derived therefrom can be used as the first antigen binding portion in the MABP of the present application. Antibodies or antibody fragments with proven clinical efficacy, safety, and pharmacokinetics profile are of particular interest. In some embodiments, the antibody or antibody fragment known in the art is further engineered, such as humanized or mutagenized to select for a variant with a suitable affinity, prior to fusion with the second antigen binding portion to provide the MABP. In some embodiments, the first antigen binding portion comprises the V_{H} and V_{L} domains of a monoclonal antibody or antibody fragment known in the art, and modified heavy chain constant region and/or light chain constant region. In some embodiments, the first antigen binding portion comprises the monoclonal antibody known in the art and a modified Fc region, such as an IgG4 Fc with an S228P mutation. In some embodiments, the first antigen binding portion comprises a human, humanized, or chimeric full-length antibody or antibody fragments.

In some embodiments, the first antigen binding portion is derived from an approved (such as by FDA and/or EMA) or investigational monoclonal antibody or antibody fragment (such as Fab). In some embodiments, the first antigen binding portion is an approved (such as by FDA and/or EMA) or investigational monoclonal antibody or antibody fragment (such as Fab).

In some embodiments, the first antigen binding portion specifically binds an immune checkpoint molecule. In some embodiments, the first antigen binding portion comprises a full-length antibody (such as antagonist antibody) or antigen binding fragment derived therefrom that specifically binds an inhibitory immune checkpoint protein. In some embodiments, the first antigen binding portion comprises a full-length antibody (such as agonist antibody) or antigen binding fragment derived therefrom that specifically binds a stimulatory checkpoint molecule. In some embodiments, the immune checkpoint molecule is selected from the group consisting of PD-1, PD-L1, PD-L2, CTLA-4, B7-H3, TIM-3, LAG-3, TIGIT, VISTA, ICOS, 4-1BB, OX40, GITR, and CD40. In some embodiments, the first antigen binding portion is an anti-PD-1 antibody or antigen binding fragment thereof. In some embodiments, the anti-PD-1 antibody is selected from the group consisting of pembrolizumab and nivolumab. In some embodiments, the first antigen binding portion is an anti-PD-L1 antibody or antigen binding fragment thereof. In some embodiments, the first antigen binding portion is an anti-TIGIT antibody or antigen binding fragment thereof. In some embodiments, the first antigen binding portion is an anti-LAG-3 antibody or antigen binding fragment thereof.

In some embodiments, the first antigen binding portion is derived from pembrolizumab. In some embodiments, the first antigen binding portion comprises pembrolizumab or antigen binding fragment thereof. In some embodiments, the first antigen binding portion comprises a VH domain comprising the amino acid sequence of SEQ ID NO: 10 and a VL domain comprising the amino acid sequence of SEQ ID NO: 11. In some embodiments, the first antigen binding portion comprises a heavy chain comprising the amino acid sequence of SEQ ID NO: 8. In some embodiments, the first antigen binding portion comprises a light chain comprising the amino acid sequence of SEQ ID NO: 9. In some embodiments, the first antigen binding portion comprises an IgG4 Fc.

Pembrolizumab (e.g., KEYTRUDA®) is a humanized antibody used in cancer immunotherapy. It targets the programmed cell death 1 (PD-1) receptor. The drug was initially used in treating metastatic melanoma. On Sep. 4, 2014 the US Food and Drug Administration (FDA) approved KEYTRUDA® under the FDA Fast Track Development Program. It is approved for use in advanced melanoma. On Oct. 2, 2015, the US FDA approved KEYTRUDA® for the treatment of metastatic non-small cell lung cancer in patients whose tumors express PD-L1 and who have failed treatments with other chemotherapeutic agents.

In some embodiments, the first antigen binding portion specifically binds a tumor antigen. In some embodiments, the tumor antigen is selected from the group consisting of HER2, BRAF, EGFR, VEGFR2, CD20, RANKL, CD38, and CD52. In some embodiments, the first antigen binding portion is an anti-HER2 antibody or antigen binding fragment thereof. In some embodiments, the first antigen binding portion is derived from trastuzumab.

Trastuzumab (HERCEPTIN®), one of the five top selling therapeutic antibodies, is a humanized anti-HER2 receptor monoclonal antibody that has significantly increased the survival rate in patients with HER2-positive breast cancer. The HER receptors are proteins that are embedded in the cell membrane and communicate molecular signals from outside the cell (molecules called EGFs) to inside the cell, and turn genes on and off. The HER protein, Human Epidermal Growth Factor Receptor, binds Human Epidermal Growth Factor, and stimulates cell proliferation. In some cancers, notably certain types of breast cancer, HER2 is over-expressed, and causes cancer cells to reproduce uncontrollably. However, among breast cancer patients, only 15-20% of them exhibit amplification and overexpression of the human epidermal growth factor receptor 2 (HER2), most HER2-patients do not respond to trastuzumab. In addition, some of the HER2+ patients have developed resistance to trastuzumab after initial treatment. As the epidermal growth factor RTK family consists of four members: EGFR, HER2, HER3 and HER4, some multispecific antibodies have been developed to target two of these antigens, which have shown advantages over conventional monospecific antibodies.

In some embodiments, the first antigen binding portion specifically binds an angiogenic factor. In some embodiments, the first antigen binding portion is an anti-Ang2 antibody or antigen binding fragment thereof. In some embodiments, the first antigen binding portion is derived from LC10.

In some embodiments, the first antigen binding portion specifically binds a pro-inflammatory molecule. In some embodiments, the pro-inflammatory molecule is selected from the group consisting of VEGF, IL-1β, TNF-α, IL-5, IL-6, IL-6R and eotaxin-1. In some embodiments, the first antigen binding portion is an anti-TNF-α antibody or antigen binding fragment thereof. In some embodiments, the first antigen binding portion is derived from adalimumab.

### Properties of the MABPs

The MABPs described herein are amenable for manufacture and development as a biologic drug. In some embodiments, the MABP can be recombinantly produced at high expression levels. In some embodiments, the MABP can be recombinantly produced at a level sufficient for industrial production. In some embodiments, the MABP can be expressed transiently in mammalian cells. In some embodiments, the MABP produced by recombinant expression can be purified to homogeneity or substantial homogeneity by a size exclusion chromatography. In some embodiments, the percentage of mono-dispersive molecule (e.g., as a monomeric MABP molecule, such as a dimeric protein consisting of 4 polypeptide chains) in the purified MABP, e.g., as determined by chromatography, is at least about any one of 90%, 91%, 92%, 93%, 94%, 95%, 96%, 97%, 98%, 99%, 99.5% or higher. The homogeneity of the MABP in a composition can be determined using known methods in the art, such as by SDS-PAGE analysis, dynamic light scattering (DLS), or analysis using an HPLC or FPLC. In some embodiments, the yield of the MABP from the purification is at least about any one of 50%, 60%, 70%, 80%, 90% or higher. In some embodiments, the yield of the MABP from the purification is about 70% to about 95%.

The MABPs described herein further has various biophysical properties that are amenable for use as a biologic drug, including, for example, high solubility, high long-term stability, and thermal stability. Stability of the MABP can be determined using known methods in the art, including Dynamic light scattering (DSL), which profiles different populations of a molecule in soluble based on their particle sizes. In some embodiments, at least about 90%, 91%, 92%, 93%, 94%, 95% or higher of the MABP in a composition is a non-aggregated conformation, i.e., as single, monomeric MABP molecules, e.g., a dimeric protein consisting of 4 polypeptide chains. In some embodiments, the level of aggregation, i.e., association of multiple MABP molecules as a complex, in a composition is no more than about any one of 1%, 2%, 3%, 4%, 5%, 6%, 7%, 8%, 9%, 10% or higher. In some embodiments, the time to form at least about 5% aggregation of the MABP in a composition is at least about any one of 1 day, 3 days, 7 days, 2 weeks, 3 weeks, 4 weeks or more at about 4° C. In some embodiments, the time to form at least about 5% aggregation of the MABP in a composition is at least about any one of 1 day, 3 days, 7 days, 2 weeks, 3 weeks, 4 weeks or more at about room temperature, e.g., 25° C. In some embodiments, the time to form at least about 10% aggregation of the MABP in a composition is at least about any one of 1 day, 2 days, 3 days, 4 days, 6 days, 7 days, 10 days, 2 weeks or more at physiological temperature, e.g., about 37° C.

In some embodiments, the MABP has comparable thermal stability as the parent 4-chain antibody or antigen-binding fragment thereof. In some embodiments, the MABP has higher thermal stability than the parent 4-chain antibodies or antigen-binding fragment thereof. Thermal stability can be measured using known methods in the art, including Capillary Differential Scanning Calorimetry (DSC) and DLS coupled to gradual heating. In some embodiments, the MABP has an aggregation onset temperature (T_{agg}) of at least about 55° C., such as at least about any one of 56° C., 57° C., 58° C., 59° C., 60° C., 61° C., 62° C., 63° C., 64° C., 65° C., 66° C., 67° C., 68° C., 69° C., 70° C. or higher. In some embodiments, the MABP has an aggregation onset temperature (T_{agg}) of about 55° C. to about 70° C.

In some embodiments, the MABP has a high long-term stability. In some embodiments, the MABP is stable for at least about any one of 1 day, 3 days, 7 days, 2 weeks, 3 week, 4 weeks or more at about 4° C. In some embodiments, the MABP has a high long-term stability at an elevated temperature. In some embodiments, the MABP is stable for at least about any one of 1 day, 3 days, 7 days, 2 weeks, 3 week, 4 weeks or more at room temperature, such as about 25° C. or higher. In some embodiments, the MABP is stable for at least about any one of 1 day, 2 days, 3 days, 4 days, 6 days, 7 days, 10 days, 2 weeks or more at physiological temperature, such as about 37° C. or higher. In some embodiments, the stability of the MABP is tested in an accelerated stability assessment program, for example, at about any one of 40° C., 50° C., 60° C., 70° C. or higher do derive the stability of the MABP at a lower temperature. In some embodiments, the MABP has a high long-term stability at a high concentration, such as at least about any one of 50 mg/mL, 100 mg/mL, 150 mg/mL, 200 mg/mL or higher. As used herein, a “stable” composition is substantially free (such as less than about any of 10%, 9%, 8%, 7%, 6%, 5%, 4%, 3%, 2%, 1% or less) of precipitation and/or aggregation. In some embodiments, the MABP has a high long-term stability in human serum for at least about any one of 1 day, 3 days, 7 days, 2 weeks, 3 week, 4 weeks or more at about 4° C. In some embodiments, the MABP has a high long-term stability in human serum for at least about any one of 1 day, 3 days, 7 days, 10 days, 2 weeks, 3 week, 4 weeks or more at physiological temperature, e.g., about 37° C. Precipitation can be detected by optical spectroscopy. Aggregation can be detected by e.g., DLS.

In some embodiments, the MABP has high stability over freeze-thaw cycles. In some embodiments, a composition comprising the MABP can be freeze-thawed for at least about any one of 3, 4, 5, 6, 7, 8, 9, 10 times or more without losing structural integrity (e.g., forming aggregates) and/or activity of the MABP. In some embodiments, the composition comprising the MABP can be freeze-thawed at high concentration, such as at least about any one of 50 mg/mL, 100 mg/mL, 150 mg/mL, 200 mg/mL or higher.

### III. Pharmaceutical Compositions

Further provided by the present application are pharmaceutical compositions comprising any one of the MABPs and a pharmaceutically acceptable carrier. Pharmaceutical compositions can be prepared by mixing a MABP having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions.

In order for the pharmaceutical compositions to be used for in vivo administration, they must be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The pharmaceutical compositions herein may also contain more than one active compound as necessary for the particular indication being treated, preferably those with complementary activities that do not adversely affect each other. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coascervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

### IV. Methods of Use

The MABPs (e.g., TABPs) described herein, and the compositions (such as pharmaceutical compositions) thereof are useful for a variety of applications, such as in diagnosis, molecular assays, and therapy.

In some embodiments, there is a method of treating a disease or a condition in an individual in need thereof, comprising administering an effective amount of a pharmaceutical composition comprising a multispecific (such as trispecific) antigen binding protein and a pharmaceutically acceptable carrier, wherein the MABP comprises: (a) a first antigen binding portion comprising a heavy chain variable domain (V_{H}) and a light chain variable domain (V_{L}), wherein the V_{H} and V_{L} together form an antigen-binding site that specifically binds a first epitope, (b) a second antigen binding portion comprising a first sdAb that specifically binds a second epitope, and (c) a third antigen binding portion comprising a second sdAb that specifically binds a third epitope, wherein the first antigen binding portion, the second antigen binding portion, and the third antigen binding portion are fused to each other. In some embodiments, the first epitope is from a first immune checkpoint molecule (e.g., PD-1, SEQ ID NO: 12), the second epitope is from a second immune checkpoint molecule (e.g., TIGIT, SEQ ID NO: 13), and the third epitope is from a third immune checkpoint molecule (e.g., LAG-3, SEQ ID NO: 14). In some embodiments, the first epitope is from a first tumor antigen, the second epitope is from a second tumor antigen, and the third epitope is from a third tumor antigen. In some embodiments, the first epitope is from a first tumor antigen (e.g., HER-2), the second epitope is from a cell surface molecule on an immune effector cell (e.g., CD3), and the third epitope is from a second tumor antigen (e.g., EGFR). In some embodiments, the first epitope is from a first pro-inflammatory molecule (e.g., TNF-α), the second epitope is from a second pro-inflammatory molecule (e.g., IL-17A), and the third epitope is from a third pro-inflammatory molecule (e.g., IL-17F). In some embodiments, the first epitope is from a first angiogenic factor (e.g., Ang2), the second epitope is from a second angiogenic factor (e.g., VEGF), and the third epitope is from a third angiogenic factor (e.g., DLL4). In some embodiments, the first sdAb and/or the second sdAb is a V_{H}H. In some embodiments, the first antigen binding portion comprises a heavy chain comprising the V_{H} and a light chain comprising the V_{L}. In some embodiments, the first antigen binding region is a full-length antibody consisting of two heavy chains and two light chains. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG1 Fc or IgG4 Fc.

### Methods of Treating a Cancer

In some embodiments, there is provided a method of treating a cancer in an individual in need thereof, comprising administering an effective amount of a pharmaceutical composition comprising a multispecific (such as trispecific) antigen binding protein and a pharmaceutically acceptable carrier, wherein the MABP comprises: (a) a first antigen binding portion comprising a heavy chain variable domain (V_{H}) and a light chain variable domain (V_{L}), wherein the V_{H} and V_{L} together form an antigen-binding site that specifically binds a first epitope, (b) a second antigen binding portion comprising a first sdAb that specifically binds a second epitope, and (c) a third antigen binding portion comprising a second sdAb that specifically binds a third epitope, wherein the first antigen binding portion, the second antigen binding portion, and the third antigen binding portion are fused to each other. In some embodiments, the cancer is selected from the group consisting of breast cancer, renal cancer, melanoma, lung cancer, glioblastoma, head and neck cancer, prostate cancer, ovarian carcinoma, bladder carcinoma, and lymphoma. In some embodiments, the first sdAb and/or the second sdAb is a V_{H}H. In some embodiments, the first antigen binding portion comprises a heavy chain comprising the V_{H} and a light chain comprising the V_{L}. In some embodiments, the first antigen binding region is a full-length antibody consisting of two heavy chains and two light chains. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG1 Fc or IgG4 Fc.

In some embodiments, there is provided a method of treating a cancer in an individual in need thereof, comprising administering an effective amount of a pharmaceutical composition comprising a multispecific (such as trispecific) antigen binding protein and a pharmaceutically acceptable carrier, wherein the MABP comprises: (a) a first antigen binding portion comprising a heavy chain variable domain (V_{H}) and a light chain variable domain (V_{L}), wherein the V_{H} and V_{L} together form an antigen-binding site that specifically binds a first immune checkpoint molecule (e.g., PD-1), (b) a second antigen binding portion comprising a first sdAb (e.g., a V_{H}H) that specifically binds a second immune checkpoint molecule (e.g., TIGIT), and (c) a third antigen binding portion comprising a second sdAb (e.g., a V_{H}H) that specifically binds a third immune checkpoint inhibitor (e.g., LAG-3), wherein the first antigen binding portion, the second antigen binding portion, and the third antigen binding portion are fused to each other. In some embodiments, the cancer is selected from the group consisting of breast cancer, renal cancer, melanoma, lung cancer, glioblastoma, head and neck cancer, prostate cancer, ovarian carcinoma, bladder carcinoma, and lymphoma. In some embodiments, the first sdAb and/or the second sdAb is a V_{H}H. In some embodiments, the first antigen binding portion comprises a heavy chain comprising the V_{H} and a light chain comprising the V_{L}. In some embodiments, the first antigen binding region is a full-length antibody consisting of two heavy chains and two light chains. In some embodiments, the first antigen binding region is derived from pembrolizumab. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG4 Fc.

In some embodiments, there is provided a method of treating a cancer in an individual in need thereof, comprising administering an effective amount of a pharmaceutical composition comprising a multispecific (such as trispecific) antigen binding protein and a pharmaceutically acceptable carrier, wherein the MABP comprises: (a) a first antigen binding portion comprising a heavy chain variable domain (V_{H}) and a light chain variable domain (V_{L}), wherein the V_{H} and V_{L} together form an antigen-binding site that specifically binds a first tumor antigen (e.g., HER-2), (b) a second antigen binding portion comprising a first sdAb (e.g., a V_{H}H) that specifically binds a cell surface antigen of an immune effector cell (e.g., CD3), and (c) a third antigen binding portion comprising a second sdAb (e.g., a V_{H}H) that specifically binds a second tumor antigen (e.g., EGFR), wherein the first antigen binding portion, the second antigen binding portion, and the third antigen binding portion are fused to each other. In some embodiments, the cancer is selected from the group consisting of breast cancer, renal cancer, melanoma, lung cancer, glioblastoma, head and neck cancer, prostate cancer, ovarian carcinoma, bladder carcinoma, and lymphoma. In some embodiments, the first sdAb and/or the second sdAb is a V_{H}H. In some embodiments, the first antigen binding portion comprises a heavy chain comprising the V_{H} and a light chain comprising the V_{L}. In some embodiments, the first antigen binding region is a full-length antibody consisting of two heavy chains and two light chains. In some embodiments, the first antigen binding region is derived from trastuzumab. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG1 Fc.

In some embodiments, there is provided a method of treating a cancer in an individual in need thereof, comprising administering an effective amount of a pharmaceutical composition comprising a multispecific (such as trispecific) antigen binding protein and a pharmaceutically acceptable carrier, wherein the MABP comprises: (a) a first antigen binding portion comprising a heavy chain variable domain (V_{H}) and a light chain variable domain (V_{L}), wherein the V_{H} and V_{L} together form an antigen-binding site that specifically binds a first angiogenic factor (e.g., Ang2), (b) a second antigen binding portion comprising a first sdAb (e.g., a V_{H}H) that specifically binds a second angiogenic factor (e.g., VEGF), and (c) a third antigen binding portion comprising a second sdAb (e.g., a V_{H}H) that specifically binds a third angiogenic factor (e.g., DLL4), wherein the first antigen binding portion, the second antigen binding portion, and the third antigen binding portion are fused to each other. In some embodiments, the cancer is selected from the group consisting of breast cancer, renal cancer, melanoma, lung cancer, glioblastoma, head and neck cancer, prostate cancer, ovarian carcinoma, bladder carcinoma, and lymphoma. In some embodiments, the first sdAb and/or the second sdAb is a V_{H}H. In some embodiments, the first antigen binding portion comprises a heavy chain comprising the V_{H} and a light chain comprising the V_{L}. In some embodiments, the first antigen binding region is a full-length antibody consisting of two heavy chains and two light chains. In some embodiments, the first antigen binding region is derived from LC10. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG1 Fc.

The methods described herein are suitable for treating various cancers, including both solid cancer and liquid cancer. The methods are applicable to cancers of all stages, including early stage, advanced stage and metastatic cancer. The methods described herein may be used as a first therapy, second therapy, third therapy, or combination therapy with other types of cancer therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

### Methods of Treating Inflammatory or Autoimmune Disease

In some embodiments, there is provided a method of treating an inflammatory or autoimmune disease in an individual in need thereof, comprising administering an effective amount of a pharmaceutical composition comprising a multispecific (such as trispecific) antigen binding protein and a pharmaceutically acceptable carrier, wherein the MABP comprises: (a) a first antigen binding portion comprising a heavy chain variable domain (V_{H}) and a light chain variable domain (V_{L}), wherein the V_{H} and V_{L} together form an antigen-binding site that specifically binds a first epitope, (b) a second antigen binding portion comprising a first sdAb that specifically binds a second epitope, and (c) a third antigen binding portion comprising a second sdAb that specifically binds a third epitope, wherein the first antigen binding portion, the second antigen binding portion, and the third antigen binding portion are fused to each other. In some embodiments, the inflammatory or autoimmune disease is selected from the group consisting of arthritis (such as rheumatoid arthritis, juvenile idiopathic arthritis, psoriatic arthritis, ankylosing spondylitis, and arthritic ulcerative colitis), colitis, psoriasis, severe asthma, and moderate to severe Crohn's disease. In some embodiments, the first sdAb and/or the second sdAb is a V_{H}H. In some embodiments, the first antigen binding portion comprises a heavy chain comprising the V_{H} and a light chain comprising the V_{L}. In some embodiments, the first antigen binding region is a full-length antibody consisting of two heavy chains and two light chains. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG1 Fc or IgG4 Fc.

In some embodiments, there is provided a method of treating an inflammatory or autoimmune disease in an individual in need thereof, comprising administering an effective amount of a pharmaceutical composition comprising a multispecific (such as trispecific) antigen binding protein and a pharmaceutically acceptable carrier, wherein the MABP comprises: (a) a first antigen binding portion comprising a heavy chain variable domain (V_{H}) and a light chain variable domain (V_{L}), wherein the V_{H} and V_{L} together form an antigen-binding site that specifically binds a first pro-inflammatory molecule (e.g., TNF-α), (b) a second antigen binding portion comprising a first sdAb that specifically binds a second pro-inflammatory molecule (e.g., IL-17A), and (c) a third antigen binding portion comprising a second sdAb that specifically binds a third pro-inflammatory molecule (e.g., IL-17F), wherein the first antigen binding portion, the second antigen binding portion, and the third antigen binding portion are fused to each other. In some embodiments, the inflammatory or autoimmune disease is selected from the group consisting of arthritis (such as rheumatoid arthritis, juvenile idiopathic arthritis, psoriatic arthritis, ankylosing spondylitis, and arthritic ulcerative colitis), colitis, psoriasis, severe asthma, and moderate to severe Crohn's disease. In some embodiments, the first sdAb and/or the second sdAb is a V_{H}H. In some embodiments, the first antigen binding portion comprises a heavy chain comprising the V_{H} and a light chain comprising the V_{L}. In some embodiments, the first antigen binding region is a full-length antibody consisting of two heavy chains and two light chains. In some embodiments, the first antigen binding region is derived from adalimumab. In some embodiments, the antigen binding portions are fused together via a peptide linker. In some embodiments, the peptide linker is no more than about 30 (such as no more than about any one of 25, 20, or 15) amino acids long. In some embodiments, the first antigen binding portion comprises an Fc region, such as an IgG1 Fc.

### Dosage and Routes of Administration

Dosages and desired drug concentrations of pharmaceutical compositions of the present application may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

When in vivo administration of the MABPs described herein are used, normal dosage amounts may vary from about 10 ng/kg up to about 100 mg/kg of mammal body weight or more per day, preferably about 1 mg/kg/day to 10 mg/kg/day, depending upon the route of administration. It is within the scope of the present application that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

In some embodiments, the pharmaceutical composition is administered for a single time. In some embodiments, the pharmaceutical composition is administered for multiple times (such as any of 2, 3, 4, 5, 6, or more times). In some embodiments, the pharmaceutical composition is administered once per week, once 2 weeks, once 3 weeks, once 4 weeks, once per month, once per 2 months, once per 3 months, once per 4 months, once per 5 months, once per 6 months, once per 7 months, once per 8 months, once per 9 months, or once per year. In some embodiments, the interval between administrations is about any one of 1 week to 2 weeks, 2 weeks to 1 month, 2 weeks to 2 months, 1 month to 2 months, 1 month to 3 months, 3 months to 6 months, or 6 months to a year. The optimal dosage and treatment regime for a particular patient can readily be determined by one skilled in the art of medicine by monitoring the patient for signs of disease and adjusting the treatment accordingly.

The pharmaceutical compositions of the present application, including but not limited to reconstituted and liquid formulations, are administered to an individual in need of treatment with the MABPs, preferably a human, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes.

In some embodiments, the pharmaceutical compositions are administered to the individual by subcutaneous (i.e. beneath the skin) administration. For such purposes, the pharmaceutical compositions may be injected using a syringe. However, other devices for administration of the pharmaceutical compositions are available such as injection devices; injector pens; auto-injector devices, needleless devices; and subcutaneous patch delivery systems.

In some embodiments, the pharmaceutical compositions are administered to the individual intravenously. In some embodiments, the pharmaceutical composition is administered to an individual by infusion, such as intravenous infusion. Infusion techniques for immunotherapy are known in the art (see, e.g., Rosenberg et al., New Eng. J. of Med. 319: 1676 (1988)).

### V. Methods of Preparation

The present application also provides isolated nucleic acids encoding the MABPs, vectors and host cells comprising such isolated nucleic acids, and recombinant methods for the production of the MABPs.

For recombinant production of the MABP, the nucleic acids encoding the full-length antibody or antigen binding fragment of the first antigen binding portion, the first sdAb and the second sdAb are isolated and inserted into a replicable vector for further cloning (amplification of the DNA) or for expression. In some embodiments, the nucleic acid encoding the full-length antibody or antigen binding fragment of the first antigen binding portion is recombinantly fused to the nucleic acid encoding the first or second sdAb and optionally via a nucleic acid encoding a peptide linker, all in frame for translation with respect to each other to provide a nucleic acid encoding the MABP. DNA encoding the MABP or components thereof is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, preferred host cells are of either prokaryotic or eukaryotic (generally mammalian) origin.

Alternatively, the first antigen binding portion, the second antigen binding portion and the third antigen binding portion are each prepared recombinantly using prokaryotic or eukaryotic host cells comprising nucleic acids that encode the first antigen binding portion, the second antigen binding portion, and the third antigen binding portion respectively. The expressed first antigen binding portion, the second antigen binding portion, and the third antigen binding portion are then conjugated chemically, and purified in order to provide the MABP.

### 1. Protein Production in Prokaryotic Cells

### a) Vector Construction

Polynucleotide sequences encoding polypeptide components of the MABP of the present application can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present application. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as GEM™-11 may be utilized in making a recombinant vector which can be used to transform susceptible host cells such as E. coli LE392.

The expression vector described herein may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the—galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20: 269) using linkers or adaptors to supply any required restriction sites.

In one aspect, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this application should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In some embodiments, the signal sequences used in both cistrons of the expression system are STII signal sequences or variants thereof.

In some embodiments, the production of the MABPs can occur in the cytoplasm of the host cell, and therefore does not require the presence of secretion signal sequences within each cistron. In some embodiments, polypeptide components are expressed, folded and assembled to form functional MABPs within the cytoplasm. Certain host strains (e.g., the E. coli trx13⁻ strains) provide cytoplasm conditions that are favorable for disulfide bond formation, thereby permitting proper folding and assembly of expressed protein subunits. Proba and Pluckthun Gene, 159:203 (1995).

The present application provides an expression system in which the quantitative ratio of expressed polypeptide components can be modulated in order to maximize the yield of secreted and properly assembled the MABPs of the present application. Such modulation is accomplished at least in part by simultaneously modulating translational strengths for the polypeptide components. One technique for modulating translational strength is disclosed in Simmons et al., U.S. Pat. No. 5,840,523. It utilizes variants of the translational initiation region (TIR) within a cistron. For a given TIR, a series of amino acid or nucleic acid sequence variants can be created with a range of translational strengths, thereby providing a convenient means by which to adjust this factor for the desired expression level of the specific chain. TIR variants can be generated by conventional mutagenesis techniques that result in codon changes which can alter the amino acid sequence, although silent changes in the nucleotide sequence are preferred. Alterations in the TIR can include, for example, alterations in the number or spacing of Shine-Dalgarno sequences, along with alterations in the signal sequence. One method for generating mutant signal sequences is the generation of a “codon bank” at the beginning of a coding sequence that does not change the amino acid sequence of the signal sequence (i.e., the changes are silent). This can be accomplished by changing the third nucleotide position of each codon; additionally, some amino acids, such as leucine, serine, and arginine, have multiple first and second positions that can add complexity in making the bank. This method of mutagenesis is described in detail in Yansura et al. (1992) METHODS: A Companion to Methods in Enzymol. 4:151-158.

Preferably, a set of vectors is generated with a range of TIR strengths for each cistron therein. This limited set provides a comparison of expression levels of each chain as well as the yield of the desired MABP products under various TIR strength combinations. TIR strengths can be determined by quantifying the expression level of a reporter gene as described in detail in Simmons et al. U.S. Pat. No. 5,840,523. Based on the translational strength comparison, the desired individual TIRs are selected to be combined in the expression vector constructs of the present application.

### b) Prokaryotic Host Cells.

Prokaryotic host cells suitable for expressing the MABPs of the present application include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In one embodiment, gram-negative cells are used. In one embodiment, E. coli cells are used as hosts. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 AfhuA (AtonA) ptr3 lac Iq lacL8 AompT A(nmpc-fepE) degP41 kan^{R} (U.S. Pat. No. 5,639,635). Other strains and derivatives thereof, such as E. coli 294 (ATCC 31,446), E. coli B, E. coli 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon.

Typically the host cell should secrete minimal amounts of proteolytic enzymes, and additional protease inhibitors may desirably be incorporated in the cell culture.

### c) Protein Production

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the MABPs of the present application are grown in media known in the art and suitable for culture of the selected host cells. Examples of suitable media include Luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector, protein expression is induced under conditions suitable for the activation of the promoter. In some embodiments, PhoA promoters are used for controlling transcription of the polypeptides. Accordingly, the transformed host cells are cultured in a phosphate-limiting medium for induction. Preferably, the phosphate-limiting medium is the C.R.A.P medium (see, e.g., Simmons et al., J. Immunol. Methods (2002), 263:133-147). A variety of other inducers may be used, according to the vector construct employed, as is known in the art.

The expressed MABPs of the present application are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Alternatively, protein production is conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (the preferred carbon/energy source). Small scale fermentation refers generally to fermentation in a fermentor that is no more than approximately 100 liters in volumetric capacity, and can range from about 1 liter to about 100 liters.

During the fermentation process, induction of protein expression is typically initiated after the cells have been grown under suitable conditions to a desired density, e.g., an OD₅₅₀ of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the MABPs of the present application, various fermentation conditions can be modified. For example, to improve the proper assembly and folding of the secreted polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive), certain host strains deficient for proteolytic enzymes can be used for the present application. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; Georgiou et al., U.S. Pat. No. 5,264,365; Georgiou et al., U.S. Pat. No. 5,508,192; Hara et al., Microbial Drug Resistance, 2:63-72 (1996).

E. coli strains deficient for proteolytic enzymes and transformed with plasmids overexpressing one or more chaperone proteins may be used as host cells in the expression system encoding the MABPs of the present application.

### d) Protein Purification

The MABPs produced herein are further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In some embodiments, Protein A immobilized on a solid phase is used for immunoaffinity purification of the MABPs comprising an Fc region described herein. Protein A is a 411 (D cell wall protein from Staphylococcus aureas which binds with a high affinity to the Fc region of antibodies. Lindmark et al (1983) J. Immunol. Meth. 62:1-13. The solid phase to which Protein A is immobilized is preferably a column comprising a glass or silica surface, more preferably a controlled pore glass column or a silicic acid column. In some applications, the column has been coated with a reagent, such as glycerol, in an attempt to prevent nonspecific adherence of contaminants. The solid phase is then washed to remove contaminants non-specifically bound to the solid phase. Finally the MABPs of interest are recovered from the solid phase by elution.

### 2. Protein Production in Eukaryotic Cells

For Eukaryotic expression, the vector components generally include, but are not limited to, one or more of the following, a signal sequence, an origin of replication, one or more marker genes, and enhancer element, a promoter, and a transcription termination sequence.

### a) Signal Sequence Component

A vector for use in a eukaryotic host may also an insert that encodes a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available.

The DNA for such precursor region is ligated in reading frame to DNA encoding the MABPs of the present application.

### b) Origin of Replication

Generally, the origin of replication component is not needed for mammalian expression vectors (the SV40 origin may typically be used only because it contains the early promoter).

### c) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up nucleic acid encoding the MABPs of the present application, such as DHFR, thymidine kinase, metallothionein-I and -II, preferably primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with the polypeptide encoding-DNA sequences, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

### d) Promoter Component

Expression and cloning vectors usually contain a promoter that is recognized by the host organism and is operably linked to the nucleic acid encoding the desired polypeptide sequences. Virtually all eukaryotic genes have an AT-rich region located approximately 25 to 30 based upstream from the site where transcription is initiated. Another sequence found 70 to 80 bases upstream from the start of the transcription of many genes is a CNCAAT region where N may be any nucleotide. The 3′ end of most eukaryotic is an AATAAA sequence that may be the signal for addition of the poly A tail to the 3′ end of the coding sequence. All of these sequences may be inserted into eukaryotic expression vectors.

Other promoters suitable for use with prokaryotic hosts include the phoA promoter, -lactamase and lactose promoter systems, alkaline phosphatase promoter, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the MABPs.

Polypeptide transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human-interferon cDNA in mouse cells under the control of a thymidine kinase promoter from herpes simplex virus. Alternatively, the Rous Sarcoma Virus long terminal repeat can be used as the promoter.

### e) Enhancer Element Component

Transcription of a DNA encoding the MABPs of the present application by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide encoding sequence, but is preferably located at a site 5′ from the promoter.

### f) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the polypeptide-encoding mRNA. One useful transcription termination component is the bovine growth hormone polyadenylation region. See WO94/11026 and the expression vector disclosed therein.

### g) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for MABP production and cultured in conventional nutrient media modified as appropriate for inducing promoters, selecting transformants, or amplifying the genes encoding the desired sequences.

### h) Culturing the Host Cells

The host cells used to produce the MABPs of the present application may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### i) Protein Purification

When using recombinant techniques, the MABPs can be produced intracellularly, in the periplasmic space, or directly secreted into the medium. If the MABP is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the MABP is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The protein composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the MABP. Protein A can be used to purify the MABPs that are based on human immunoglobulins containing 1, 2, or 4 heavy chains (Lindmark et al., J. Immunol. Meth. 62:1-13 (1983)). Protein G is recommended for all mouse isotypes and for human 3 (Guss et al., EMBO J. 5:15671575 (1986)). The matrix to which the affinity ligand is attached is most often agarose, but other matrices are available. Mechanically stable matrices such as controlled pore glass or poly(styrene-divinyl)benzene allow for faster flow rates and shorter processing times than can be achieved with agarose. Where the MABP comprises a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the MABP to be recovered.

Following any preliminary purification step(s), the mixture comprising the MABP of interest and contaminants may be subjected to low pH hydrophobic interaction chromatography using an elution buffer at a pH between about 2.5-4.5, preferably performed at low salt concentrations (e.g., from about 0-0.25M salt).

### 3. Antibody Production

Components of the MABPs, such as conventional 4-chain antibodies, antigen-binding fragments, and sdAbs, can be produced using any known methods in the art, including methods described below.

The sdAbs (such as V_{H}Hs) may be obtained using methods known in the art such as by immunizing a Camelidae species (such as camel or llama) and obtaining hybridomas therefrom, or by cloning a library of sdAbs using molecular biology techniques known in the art and subsequent selection by ELISA with individual clones of unselected libraries or by using phage display.

### 1) Monoclonal Antibodies

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986).

The immunizing agent will typically include the antigenic protein or a fusion variant thereof. Generally either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridotna cell. Goding, Monoclonal Antibodies: Principles and Practice, Academic. Press (1986), pp. 59-103.

Immortalized cell lines are usually transformed mammalian cells, particularly myeloma cells of rodent, bovine and human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors available from the Salk Institute Cell Distribution Center, San Diego, Calif. USA, and SP-2 cells (and derivatives thereof. e.g., X63-Ag8-653) available from the American Type Culture Collection, Manassas, Va. USA. Human myeloma and mouse-human heteromyeloma cell lines also have been described for the production of human monoclonal antibodies (Kozbor, J. Immunol., 133:3001 (1984): Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed against the desired antigen. Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, DMEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5:256-262 (1993) and Pliickthun, Immunol. Revs. 130:151-188 (1992).

In a further embodiment, antibodies can be isolated from antibody phage libraries generated using the techniques described in McCafferty et al., Nature, 348:552-554 (1990). Clackson et al., Nature. 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### 2) Humanized Antibodies

The antibodies may further comprise humanized or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domain, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. Jones et al., Nature 321: 522-525 (1986); Riechmann et al., Nature 332: 323-329 (1988) and Presta, Curr. Opin. Struct. Biol. 2: 593-596 (1992).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers, Jones et al., Nature 321:522-525 (1986); Riechmann et al., Nature 332:323-327 (1988); Verhoeyen et al., Science 239:1534-1536 (1988), or through substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from anon-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody. Sims et al., J. Immunol., 151:2296 (1993); Chothia et al., J. Mol. Biol., 196:901 (1987). Another method uses a particular framework derived from the consensus sequence of all human antibodies of a particular subgroup of light or heavy chains. The same framework may be used for several different humanized antibodies. Carter et al., Proc. Natl. Acad. Sci. USA, 89:4285 (1992); Presta et al., J. Immunol., 151:2623 (1993).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Various forms of the humanized antibody are contemplated. For example, the humanized antibody may be an antibody fragment, such as an Fab, which is optionally conjugated with one or more cytotoxic agent(s) in order to generate an immunoconjugate. Alternatively, the humanized antibody may be an intact antibody, such as an intact IgG1 antibody.

In some embodiments, the first sdAb and/or the second sdAb is modified, such as humanized, without diminishing the native affinity of the domain for antigen and while reducing its immunogenicity with respect to a heterologous species. For example, the amino acid residues of the antibody variable domain (V_{H}H) of an llama antibody can be determined, and one or more of the Camelidae amino acids, for example, in the framework regions, are replaced by their human counterpart as found in the human consensus sequence, without that polypeptide losing its typical character, i.e. the humanization does not significantly affect the antigen binding capacity of the resulting polypeptide. Humanization of Camelidae sdAbs requires the introduction and mutagenesis of a limited amount of amino acids in a single polypeptide chain. This is in contrast to humanization of scFv, Fab′, (Fab′)₂ and IgG, which requires the introduction of amino acid changes in two chains, the light and the heavy chain and the preservation of the assembly of both chains.

### 3) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); U.S. Pat. No. 5,591,669 and WO 97/17852. Transgenic mice or rats capable of producing fully human sdAbs are known in the art. See, e.g., US20090307787A1, U.S. Pat. No. 8,754,287, US20150289489A1, US20100122358A1, and WO2004049794.

Alternatively, phage display technology can be used to produce human antibodies and antibody fragments in vitro, from immunoglobulin variable (V) domain gene repertoires from unimmunized donors. McCafferty et al., Nature 348:552-553 (1990); Hoogenboom and Winter, J. Mol. Biol. 227: 381 (1991). According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S and Chiswell, David J., Curr. Opin Struct. Biol. 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See also, U.S. Pat. Nos. 5,565,332 and 5,573,905.

The techniques of Cole et al., and Boemer et al., are also available for the preparation of human monoclonal antibodies (Cole et al., Monoclonal Antibodies and Cancer Therapy, Alan R. Liss, p. 77 (1985) and Boemer et al., J. Immunol. 147(1): 86-95 (1991). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly and antibody repertoire. This approach is described, for example, in U.S. Pat. Nos. 5,545,807; 5,545,806, 5,569,825, 5,625,126, 5,633,425, 5,661,016 and in the following scientific publications: Marks et al., Bio/Technology 10: 779-783 (1992); Lonberg et al., Nature 368: 856-859 (1994); Morrison, Nature 368: 812-13 (1994), Fishwild et al., Nature Biotechnology 14: 845-51 (1996), Neuberger, Nature Biotechnology 14: 826 (19%) and Lonberg and Huszar, Intern. Rev. Immunol. 13: 65-93 (1995).

Finally, human antibodies may also be generated in vitro by activated B cells (see U.S. Pat. Nos. 5,567,610 and 5,229,275).

### 4) Antibody Fragments

In certain circumstances there are advantages to using antibody fragments, such as antigen binding fragments, rather than whole antibodies. Smaller fragment sizes allow for rapid clearance, and may lead to improved access to solid tumors.

Various techniques have been developed for the production of antibody fragments. Traditionally, these fragments were derived via proteolytic digestion of intact antibodies (see, e.g., Morimoto et al., J Biochem Biophys. Method 24:107-117 (1992): and Brennan et al., Science 229:81 (1985)). However, these fragments can now be produced directly by recombinant host cells. Fab, Fv and scFv antibody fragments can all be expressed in and secreted from E. coli, thus allowing the facile production of large amounts of these fragments. Antibody fragments can be isolated from the antibody phage libraries discussed above. Alternatively. Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ with increase in vivo half-life is described in U.S. Pat. No. 5,869,046. In other embodiments, the antibody of choice is a single chain Fv fragment (scFv). See WO 93/16185; U.S. Pat. Nos. 5,571,894 and 5,587,458. The antibody fragment may also be a “linear antibody”. e.g., as described in U.S. Pat. No. 5,641,870. Such linear antibody fragments may be monospecific or bispecific.

### 5) Multispecific Antibodies

The first antigen binding portion may comprise a multispecific antibody, such as a bispecific antibody. Bispecific antibodies (BsAbs) are antibodies that have binding specificities for at least two different epitopes, including those on the same or another protein. Alternatively, one arm can bind the target antigen, and another arm can be combined with an arm that binds a triggering molecule on a leukocyte such as a T-cell receptor molecule (e.g., CD3), or Fc receptors for IgG (FcγR) such as FcγR1 (CD64), FcγRII (CD32) and FcγRIII (CD16), so as to focus and localize cellular defense mechanisms to the target antigen-expressing cell. Such antibodies can be derived from full length antibodies or antibody fragments (e.g. F(ab′)₂ bispecific antibodies).

Bispecific antibodies may also be used to localize cytotoxic agents to cells which express the target antigen. Such antibodies possess one arm that binds the desired antigen and another arm that binds the cytotoxic agent (e.g., saporin, anti-interferon-α, vinca alkoloid, ricin A chain, methotrexate or radioactive isotope hapten). Examples of known bispecific antibodies include anti-ErbB2/anti-FcgRIII (WO 96/16673), anti-ErbB2/anti-FcgRI (U.S. Pat. No. 5,837,234), anti-ErbB2/anti-CD3 (U.S. Pat. No. 5,821,337).

Methods for making bispecific antibodies are known in the art. Traditional production of full length bispecific antibodies is based on the coexpression of two immunoglobulin heavy-chain/light chain pairs, where the two chains have different specificities. Millstein et al., Nature, 305:537-539 (1983). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829 and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, C_{H}2, and C_{H}3 regions. It is preferred to have the first heavy-chain constant region (C_{H}1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecules provides for an easy way of separation. This approach is disclosed in WO 94/04690. For further details of generating bispecific antibodies, see, for example, Suresh et al., Methods in Enzymology 121: 210 (1986).

According to another approach described in WO 96/27011 or U.S. Pat. No. 5,731,168, the interface between a pair of antibody molecules can be engineered to maximize the percentage of heterodimers which are recovered from recombinant cell culture. The preferred interface comprises at least a part of the C_{H}3 region of an antibody constant domain. In this method, one or more small amino acid side chains from the interface of the first antibody molecule are replaced with larger side chains (e.g., tyrosine or trptophan). Compensatory “cavities” of identical or similar size to the large side chains(s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. Brennan et al., Science 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175: 217-225 (1992) describes the production of fully humanized bispecific antibody F(ab′)₂ molecules. Each Fab′ fragment was separately secreted from E. coli and subjected to directed chemical coupling in vitro to form the bispecific antibody. The bispecific antibody thus formed was able to bind cells overexpressing the ErbB2 receptor and normal human T cells, as well as trigger the lytic activity of human cytotoxic lymphocytes against human breast tumor targets.

Various techniques for making and isolating bivalent antibody fragments directly from recombinant cell culture have also been described. For example, bivalent heterodimers have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. The “diabody” technology described by Hollinger et al., Proc. Nat. Acad. Sci. USA, 90: 6444-6448 (1993) has provided an alternative mechanism for making bispecific/bivalent antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific/bivalent antibody fragments by the use of single-chain Fv (scFv) dimers has also been reported. See Gruber et al., J. Immunol., 152:5368 (1994).

Antibodies with more than two valencies are contemplated. For example, trispecific antibodies can be prepared. Tutt et al., J. Immunol. 147: 60 (1991).

Exemplary bispecific antibodies may bind two different epitopes on a given molecule. Alternatively, an anti-protein arm may be combined with an arm which binds a triggering molecule on a leukocyte such as a T-cell receptor molecule (e.g., CD2, CD3, CD28 or B7), or Fc receptors for IgG (FcγR), such as FcγRI (CD64), FcγRII (CD32) and FcγRII(CD16) so as to focus cellular defense mechanisms to the cell expressing the particular protein. Bispecific antibodies may also be used to localize cytotoxic agents to cells which express a particular protein. Such antibodies possess a protein-binding arm and an arm which binds a cytotoxic agent or a radionuclide chelator, such as EOTUBE, DPTA, DOTA or TETA. Another bispecific antibody of interest binds the protein of interest and further binds tissue factor (TF).

### 6) Multivalent Antibodies

The first antigen binding portion may comprise a multivalent antibody. A multivalent antibody may be internalized (and/or catabolized) faster than a bivalent antibody by a cell expressing an antigen to which the antibodies bind. The antibodies used as the first antigen binding portion in the MABPs of the present application can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X₂)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: V_{H}-C_{H}1-flexible linker-V_{H}-C_{H}1-Fc region chain; or V_{H}-C_{H}1-V_{H}-C_{H}1A-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a C_{L} domain.

### 7) Heteroconjugate Antibodies

Heteroconjugate antibodies can also be used as the first antigen binding portion of the MABPs of the present application. Heteroconjugate antibodies are composed of two covalently joined antibodies. For example, one of the antibodies in the heteroconjugate can be coupled to avidin, the other to biotin. Such antibodies have, for example, been proposed to target immune system cells to unwanted cells, U.S. Pat. No. 4,676,980, and for treatment of HIV infection. WO 91/00360. WO 92/200373 and EP 0308936. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980. Heteroconjugate antibodies may be made using any convenient cross-linking methods. Suitable cross-linking agents are well known in the art, and are disclosed in U.S. Pat. No. 4,676,980, along with a number of cross-linking techniques.

### 8) Effector Function Engineering

It may be desirable to modify the MABPs of the present application with respect to Fc effector function. e.g., so as to modify (e.g., enhance or eliminate) antigen-dependent cell-mediated cytotoxicity (ADCC) and/or complement dependent cytotoxicity (CDC) of the antibody. In a preferred embodiment. Fc effector function of the MABP is reduced or eliminated. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric MABP thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989).

To increase the serum half-life of the antibody, one may incorporate a salvage receptor binding epitope into the MABP as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

### 9) Other Amino Acid Sequence Modifications

Amino acid sequence modification(s) of the antibodies, such as single chain antibodies or antibody components of the MABPs, described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

A useful method for identification of certain residues or regions of the antibody that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in the Table 2 below under the heading of “preferred substitutions”. If such substitutions result in a change in biological activity, then more substantial changes, denominated “exemplary substitutions” in Table 2, or as further described below in reference to amino acid classes, may be introduced and the products screened.

TABLE 2
Amino Acid Substitutions

| Origlnal Residue | Exemplary Substitutions | Preferred |
|---|---|---|
| | | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; gln | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | Ala | ala |
| Ser (S) | Thr | thr |
| Thr (T) | Ser | ser |
| Trp (W) | tyr; phe | tyr |
| Tyr (Y) | trp; phe; thr; ser | phe |
| Val (V) | ile; leu; met; phe; ala; norleucine | leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;
(2) neutral hydrophilic: cys, ser, thr;
(3) acidic: asp, glu;
(4) basic: asn, gin, his, lys, arg;
(5) residues that influence chain orientation: gly, pro; and
(6) aromatic: trp, tyr, phe.

Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and its target (e.g., PD-L1, B7.1). Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants to the MABPs of the present application are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version.

### 10) Other Modifications

The MABPs of the present application can be further modified to contain additional nonproteinaceous moieties that are known in the art and readily available. Preferably, the moieties suitable for derivatization of the antibody are water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc. Such techniques and other suitable formulations are disclosed in Remington: The Science and Practice of Pharmacy. 20th Ed., Alfonso Gennaro, Ed., Philadelphia College of Pharmacy and Science (2000).

### VI. Kits and Articles of Manufacture

Further provided are kits, unit dosages, and articles of manufacture comprising any of the MABPs described herein. In some embodiments, a kit is provided comprising any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

### EXAMPLES

The examples below are intended to be purely exemplary of the invention and should therefore not be considered to limit the invention in any way. The following examples and detailed description are offered by way of illustration and not by way of limitation.

### Example 1: Construction and Expression of PD-1/TIGIT/LAG-3 Trispecific Antigen Binding Proteins

This example describes the construction and expression of exemplary PD-1/TIGIT/LAG-3 trispecific antigen binding proteins (TABPs). 10 constructs (TPTL11-TPTL20) were designed and expressed, each comprising two polypeptides as follows. The anti-PD-1 antibody is derived from pembrolizumab. The anti-TIGIT V_{H}H is derived from AS19584VH28 (SEQ ID NO: 31). The anti-LAG-3 V_{H}H is derived from VHH2 (SEQ ID NO: 32).

TPTL11: The first polypeptide comprises from the N-terminus to the C terminus: the anti-TIGIT V_{H}H, a peptide linker and a heavy chain of the anti-PD-1 antibody; and the second polypeptide comprises from the N-terminus to the C terminus: the anti-LAG-3 V_{H}H, a peptide linker and a light chain of the anti-PD-1 antibody. See, FIG. 1.

TPTL12: The first polypeptide comprises from the N-terminus to the C terminus: the anti-TIGIT V_{H}H, a peptide linker, the anti-LAG-3 V_{H}H, a peptide linker, and a heavy chain of the anti-PD-1 antibody; and the second polypeptide comprises a light chain of the anti-PD-1 antibody. See, FIG. 2.

TPTL13: The first polypeptide comprises a heavy chain of the anti-PD-1 antibody; and the second polypeptide comprises from the N-terminus to the C terminus: the anti-TIGIT V_{H}H, a peptide linker, the anti-LAG-3 V_{H}H, a peptide linker, and a light chain of the anti-PD-1 antibody. See, FIG. 3.

TPTL14: The first polypeptide comprises a heavy chain of the anti-PD-1 antibody; and the second polypeptide comprises from the N-terminus to the C terminus: the anti-TIGIT V_{H}H, a peptide linker, a light chain of the anti-PD-1 antibody, a peptide linker, and the anti-LAG-3 V_{H}H. See, FIG. 4.

TPTL15: The first polypeptide comprises from the N-terminus to the C terminus: the anti-TIGIT V_{H}H, a peptide linker, and a heavy chain of the anti-PD-1 antibody; and the second polypeptide comprises from the N-terminus to the C terminus: a light chain of the anti-PD-1 antibody, a peptide linker, and the anti-LAG-3 V_{H}H. See, FIG. 5.

TPTL16: The first polypeptide comprises from the N-terminus to the C terminus: the anti-TIGIT V_{H}H, a peptide linker, a heavy chain of the anti-PD-1 antibody, a peptide linker, and the anti-LAG-3 V_{H}H; and the second polypeptide comprises a light chain of the anti-PD-1 antibody. See, FIG. 6.

TPTL17: The first polypeptide comprises from the N-terminus to the C terminus: a heavy chain of the anti-PD-1 antibody, a peptide linker, and the anti-LAG-3 V_{H}H; and the second polypeptide comprises from the N-terminus to the C terminus: the anti-TIGIT V_{H}H, a peptide linker, and a light chain of the anti-PD-1 antibody. See, FIG. 7.

TPTL18: The first polypeptide comprises from the N-terminus to the C terminus: a heavy chain of the anti-PD-1 antibody, a peptide linker, and the anti-TIGIT V_{H}H; and the second polypeptide comprises from the N-terminus to the C terminus: a light chain of the anti-PD-1 antibody, a peptide linker and the anti-LAG-3 V_{H}H. See, FIG. 8.

TPTL19: The first polypeptide comprises a heavy chain of the anti-PD-1 antibody; and the second polypeptide comprises from the N-terminus to the C terminus: a light chain of the anti-PD-1 antibody, a peptide linker, the anti-LAG-3 V_{H}H, a peptide linker and the anti-TIGIT V_{H}H. See, FIG. 9.

TPTL20: The first polypeptide comprises from the N-terminus to the C terminus: a heavy chain of the anti-PD-1 antibody, a peptide linker, the anti-LAG-3 V_{H}H, a peptide linker and the anti-TIGIT V_{H}H; and the second polypeptide comprises a light chain of the anti-PD-1 antibody. See, FIG. 10.

Each TABP consists of two chains of the first polypeptide and two chains of the second polypeptide. An S228P mutation was introduced to the IgG4 Fc region to inhibit Fab arm exchange. Furthermore, the Fc region of the TABP may be swapped with IgG Fc of a different isotype, for example, the IgG1 isotype. The Fc region of IgG4 isotype has low binding affinity to FcγRs, and thus is preferable over IgG1 isotype in some embodiments for avoiding ADCC-mediated depletion of PD-1, TIGIT or LAG-3 positive cells.

CHO-K1 cells expressing each of the 10 PD-1/TIGIT/LAG-3 TABP constructs were generated. CHO-K1 cells were used to express the TABPs, which were purified by chromatography through a column containing Protein A agarose resin followed by a size exclusion column. Data related to the production of the 10 TABPs (TPTL11-TPTL20) is summarized in FIG. 11. The amino acid sequences of exemplary TABPs are provided in Table 3.

### Example 2: Binding Affinity of PD-1/TIGIT/LAG-3 TABPs

After purification, the binding affinity parameters of the TABPs were measured and compared with the corresponding monospecific antibodies (e.g., anti-PD-1 antibody pembrolizumab (KEYTRUDA®), anti-TIGIT AS19584VH28 HCAb (SEQ ID NO: 29), or anti-LAG-3 VHH2 HCAb (SEQ ID NO:30)).

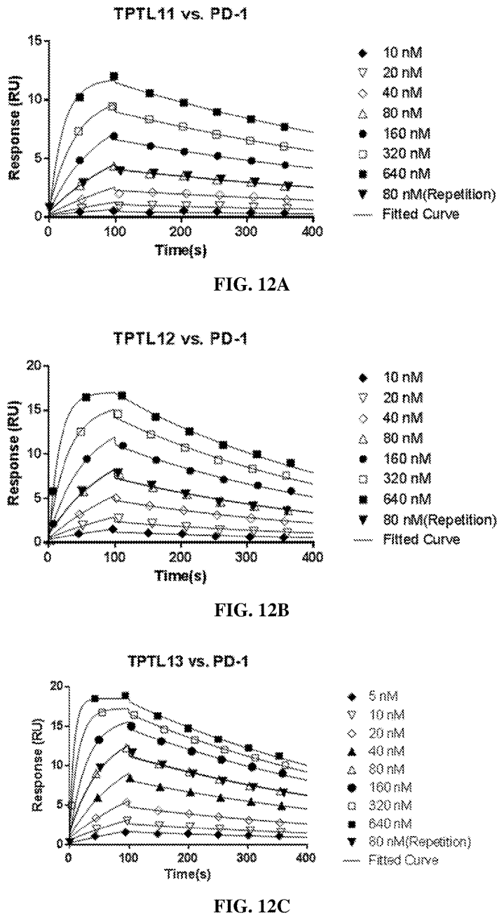

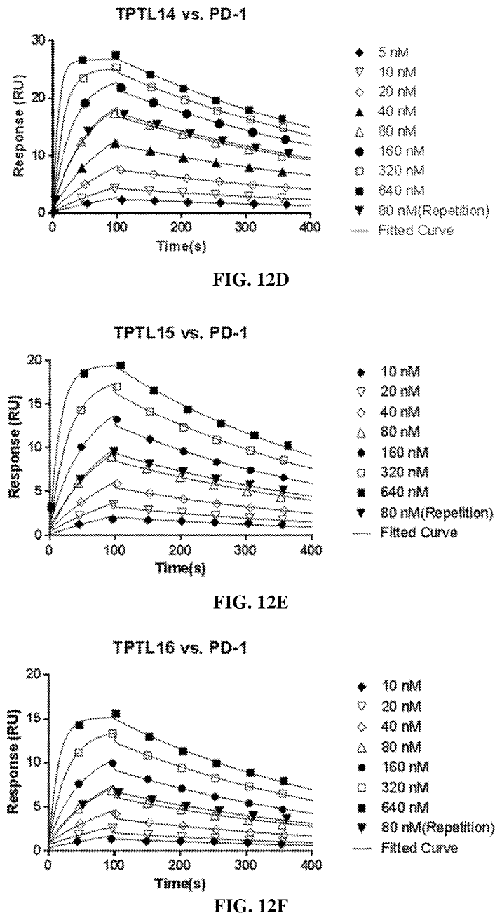

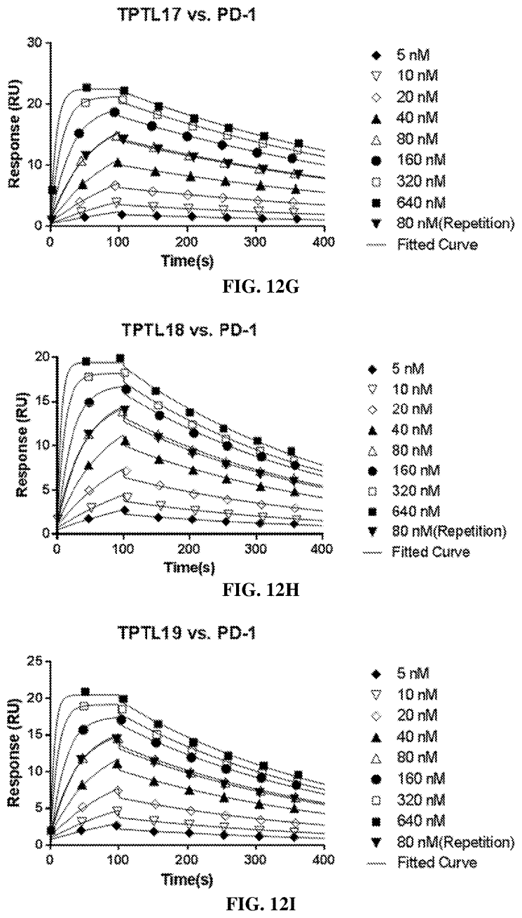

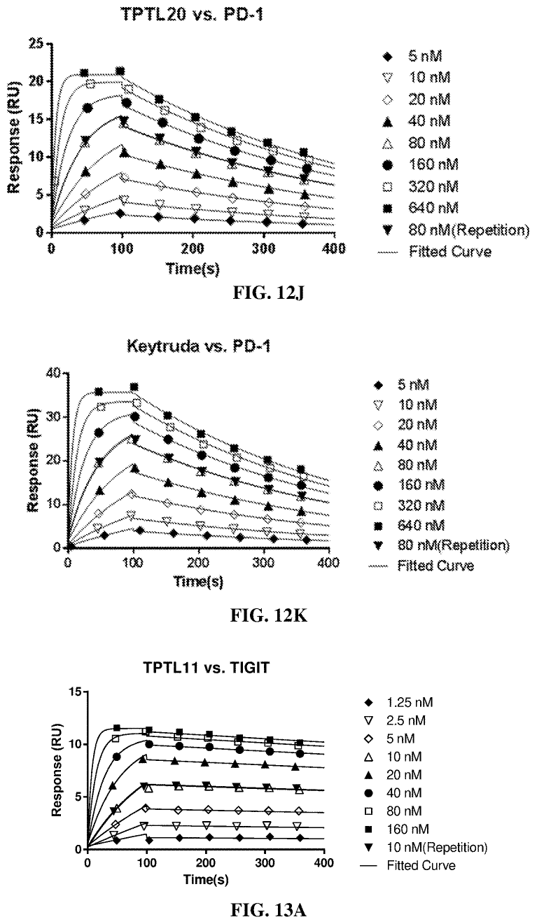

Briefly, to determine binding affinity to PD-1, each TABP or pembrolizumab was captured onto a BIACORE® chip through an anti-human Fc antibody and a His-tagged PD-1 protein was flown over the chip as the analyte at concentrations of 5, 10, 20, 40, 80, 160, 320 and 640 nM respectively. Binding curves at different analyte concentrations were used to calculate the kinetic parameters kₒₙ, k_{off} and K_{d} (FIGS. 12A-12K).

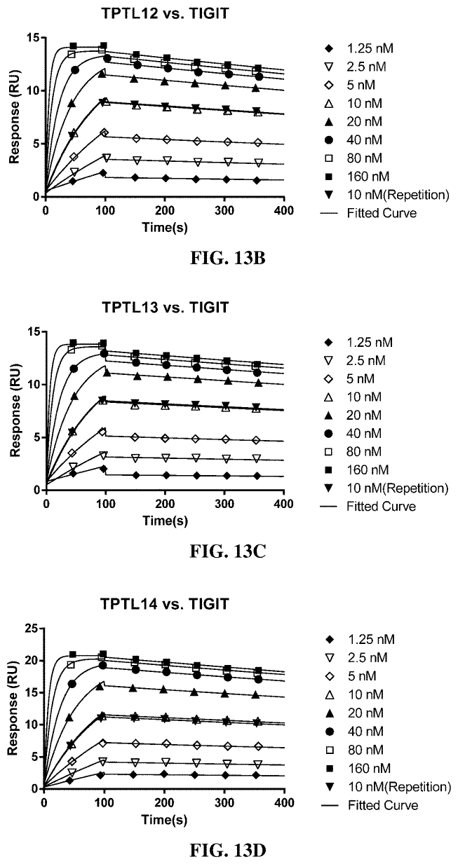

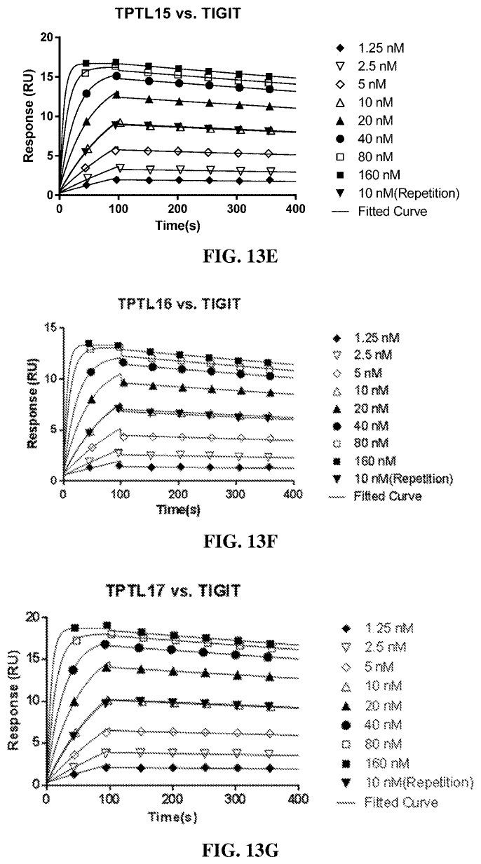

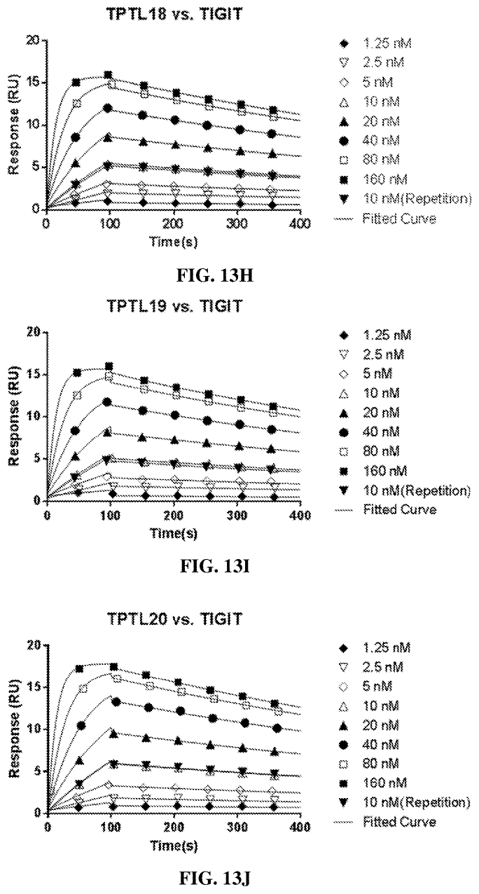

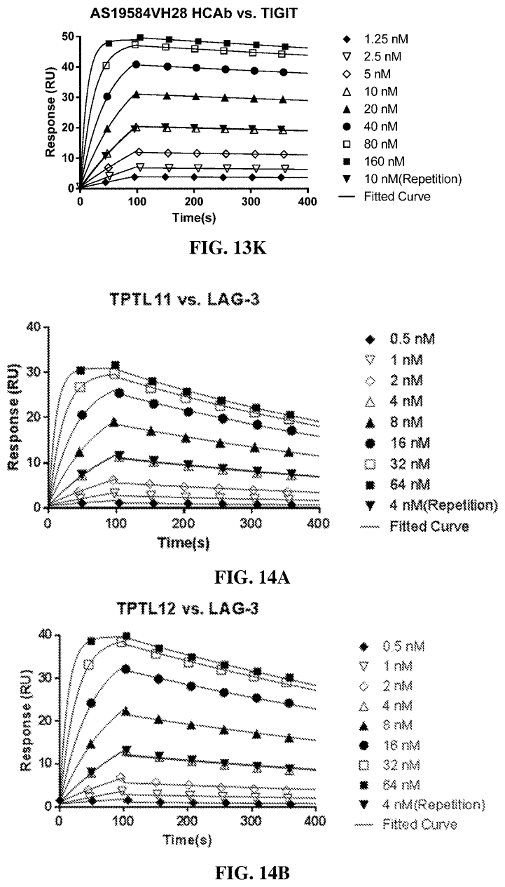

To determine binding affinity to TIGIT, each TABP or AS19584VH28 HCAb was captured onto a BIACORE® chip through an anti-human Fc antibody, and a His-tagged TIGIT protein was flown over the chip as the analyte at concentrations of 1.25, 2.5, 5, 10, 20, 40, 80 and 160 nM respectively. Binding curves at different analyte concentrations were used to calculate the kinetic parameters kₒₙ, k_{off} and K_{d} (FIGS. 13A-13K).

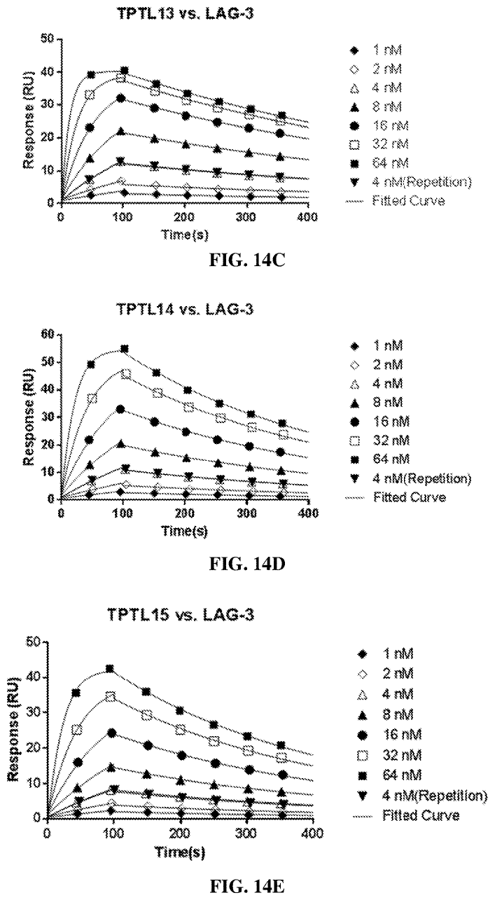

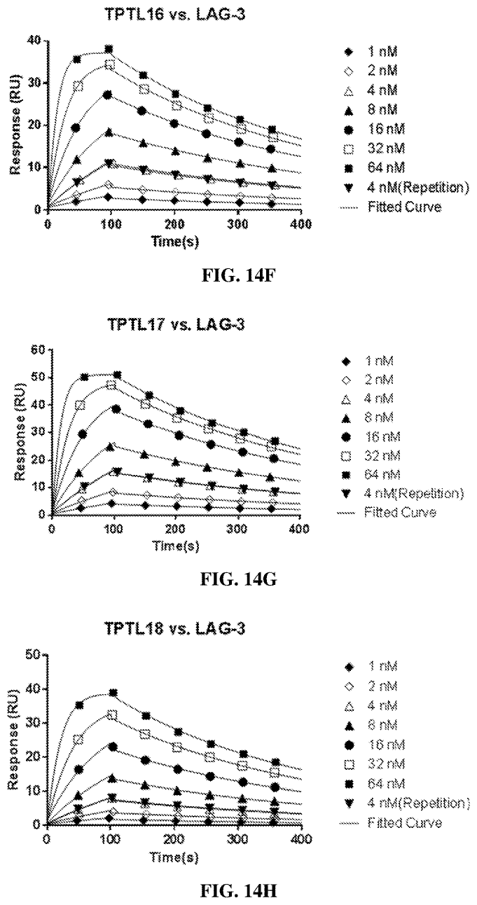

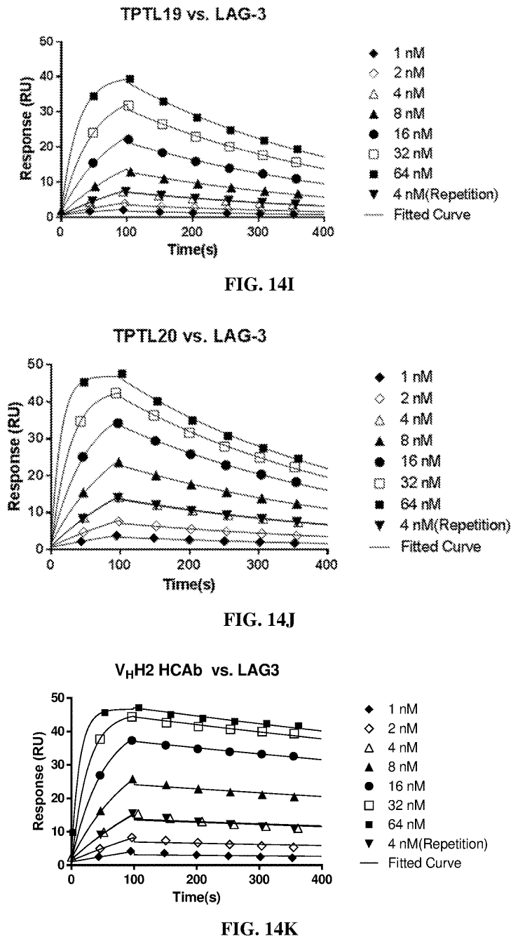

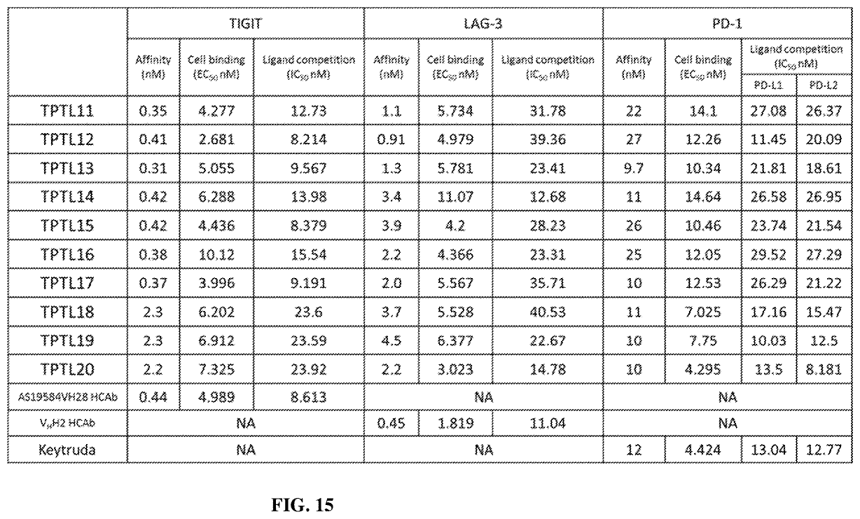

To determine binding affinity to LAG-3, each TABP or VHH2 HCAb was immobilized onto a BIACORE® chip, and a His-tagged LAG-3 protein was flown over the chip as analyte at concentrations of 1.56, 3.125, 6.25, 12.5, 25 and 50 nM respectively. Binding curves at different analyte concentrations were used to calculate the kinetic parameters kₒₙ, k_{off} and K_{d} (FIGS. 14A-14K). The calculated K_{d} shown as affinity was listed in the FIG. 15. Comparing with parent antibody, TPTL-11 to TPTL-17 has comparable affinities to Keytruda. While the affinities of most of the TABPs for TIGIT and LAG-3 are within 4 fold.

### Example 3: FACS-Based Characterization of PD-1/TIGIT/LAG-3 TABPs

The PD-1/TIGIT/LAG-3 TABPs prepared in Example 1 were tested in a FACS-based assay described below to assess their target binding ability against PD-1, TIGIT and LAG-3.

### Target Binding

Binding of PD-1/TIGIT/LAG-3 TABPs (TPTL11-TPTL20) to human PD-1 expressed on CHO cells was determined using a FACS-based assay. CHO cells expressing human PD-1 were dissociated from adherent culture flasks and mixed with varying concentrations of each TABP, pembrolizumab (KEYTUDA® as positive control), or human IgG (as negative control) in 96-well plates. The mixture was equilibrated for 30 minutes at room temperature, and washed three times with FACS buffer (PBS containing 1% BSA). FITC-conjugated anti-human Fc antibody (Jackson ImmunoResearch) used as secondary antibody was then added and the mixture was incubated for 15 minutes at room temperature. Cells were washed again with FACS buffer and analyzed by flow cytometry. Data were analyzed with PRISM™ (GraphPad Software, San Diego, Calif.) using non-linear regression, and EC₅₀ values were calculated. As shown in FIG. 15, the FACS binding assay demonstrated that TPTL11-TPTL20 exhibited comparable PD-1 binding ability as pembrolizumab (KEYTUDA®), wherein the EC₅₀ values were within 4 fold.

Binding of PD-1/TIGIT/LAG-3 TABPs (TPTL11-TPTL20) to human TIGIT expressed on CHO cells was determined using a FACS-based assay. CHO cells expressing human TIGIT were dissociated from adherent culture flasks and mixed with varying concentrations of each TABP, AS19584VH28 HCAb (as positive control), or human IgG (as negative control) in 96-well plates. The mixture was equilibrated for 30 minutes at room temperature, and washed three times with FACS buffer (PBS containing 1% BSA). FITC-conjugated anti-human Fc antibody (Jackson ImmunoResearch) used as secondary antibody was then added and the mixture was incubated for 15 minutes at room temperature. Cells were washed again with FACS buffer and analyzed by flow cytometry. Data were analyzed with PRISM™ (GraphPad Software, San Diego, Calif.) using non-linear regression, and EC₅₀ values were calculated. As shown in FIG. 15, the FACS binding assay demonstrated that TPTL11-TPTL20 exhibited comparable TIGIT binding ability as AS19584VH28 HCAb, wherein the EC₅₀ values were within 2 fold.

Binding of PD-1/TIGIT/LAG-3 TABPs (TPTL11-TPTL20) to human LAG-3 expressed on CHO cells was determined using a FACS-based assay. CHO cells expressing human LAG-3 were dissociated from adherent culture flasks and mixed with varying concentrations of each TABP, VHH2 HCAb (as positive control), or human IgG (as negative control) in 96-well plates. The mixture was equilibrated for 30 minutes at room temperature, and washed three times with FACS buffer (PBS containing 1% BSA). FITC-conjugated anti-human Fc antibody (Jackson ImmunoResearch) used as secondary antibody was then added and incubated for 15 minutes at room temperature. Cells were washed again with FACS buffer and analyzed by flow cytometry. Data was analyzed with PRISM™ (GraphPad Software, San Diego, Calif.) using non-linear regression, and EC₅₀ values were calculated. As shown in FIG. 15, the FACS binding assay demonstrated that most TABPs (TPTL11-TPTL13, TPTL15-TPTL18 and TPTL20) exhibited comparable LAG-3 binding ability as VHH2 HCAb, wherein the EC₅₀ values were within 4 fold. The binding affinities of TPTL14 and TPTL19 to LAG-3 were 5-fold weaker than VHH2 HCAb.

### Inhibition of Ligand Binding

Inhibition of ligand binding by the TABPs was also assessed by a FACS assay. To assess inhibition of PD-L1 and PD-L2 binding to PD-1 by the TABPs (TPTL11-TPTL20), CHO cells expressing human PD-1 were dissociated from adherent culture flasks and mixed with varying concentrations of each TABP and a constant concentration of hPD-L1 Fc or PD-L2 Fc fusion protein having a biotin label. Each mixture was equilibrated for 30 minutes at room temperature, and washed three times with FACS buffer (PBS containing 1% BSA). PE/Cy5 Streptavidin secondary antibody was then added to each mixture and incubated for 15 minutes at room temperature. Subsequently, the cells were washed with FACS buffer and analyzed by flow cytometry. Data was analyzed with PRISM™ (GraphPad Software, San Diego, Calif.) using non-linear regression, and IC₅₀ values were calculated. As shown in FIG. 15, the competition assay demonstrated the ability of the TABPs to efficiently inhibit PD-1/PD-L1 and PD-1/PD-L2 interaction at low concentrations (1-10 μg/ml). All 10 TABPs had comparable ligand blocking activities with IC₅₀ values within 3 fold.

To assess inhibition of CD155 binding to TIGIT by the TABPs (TPTL11-TPTL20), CHO cells expressing human TIGIT cells were dissociated from adherent culture flasks and mixed with varying concentrations of each TABP and a constant concentration of hCD155 Fc fusion protein having a biotin-label. Each mixture was equilibrated for 30 minutes at room temperature, and washed three times with FACS buffer (PBS containing 1% BSA). PE/Cy5 Streptavidin secondary antibody was then added to each mixture and incubated for 15 minutes at room temperature. Subsequently, the cells were washed again with FACS buffer and analyzed by flow cytometry. Data was analyzed with PRISM™ (GraphPad Software, San Diego, Calif.) using non-linear regression, and IC₅₀ values were calculated. As shown in FIG. 15, the competition assay demonstrated the ability of the TABPs to efficiently inhibit TIGIT-CD155 interaction at low concentrations (1-10 μg/ml). All 10 TABPs had comparable ligand blocking activities with IC₅₀ values within 3 fold. TPTL11-TPTL17 exhibited stronger inhibitory activities than TPTL18-TPTL20, which suggests that fusion to the C-terminus of the heavy chain of the anti-PD-1 antibody may inhibit binding of the anti-TIGIT sdAb to its target.

To assess inhibition of LAG-3 ligand binding by the TABPs (TPTL11-TPTL20), A375 cells expressing human TIGIT ligand (MHC class II) were dissociated from adherent culture flasks and mixed with varying concentrations of each TABP and a constant concentration of LAG-3 Fc fusion protein having a biotin-label. Each mixture was equilibrated for 30 minutes at room temperature, and washed three times with FACS buffer (PBS containing 1% BSA). PE/Cy5 Streptavidin secondary antibody was then added to each mixture and incubated for 15 minutes at room temperature. Subsequently, the cells were washed again with FACS buffer and analyzed by flow cytometry. Data was analyzed with PRISM™ (GraphPad Software, San Diego, Calif.) using non-linear regression, and IC₅₀ values were calculated. As shown in FIG. 15, the competition assay demonstrated the ability of the TABPs to efficiently inhibit LAG-3-MHC II interaction at low concentrations (1-10 μg/ml). TPTL12, TPTL17 and TPTL18 showed reduced inhibitory activity against LAG-3 ligand binding.

### Example 4: Developability of PD-1/TIGIT/LAG-3 TABPs

The PD-1/TIGIT/LAG-3 TABPs prepared in Example 1 were tested in a developability assay described below to assess their stability.

### The Onset Aggregation Temperature (Tagg)

A temperature ramp from 25° C. to 80° C. with temperature interval at about 0.75° C. was performed for samples at 5 mg/ml using the DynaPro NanoStar (Wyatt, Santa Barbara, Calif.). 20 μl of each protein samples was added to Wyatt Disposable Cuvette followed by covering the sample with 10 μl of mineral oil (Sigma 8410) to prevent evaporation. Triplicate measurements (5 acquisitions/each measurement) were averaged for each protein sample.

In the duration of an experiment with the chosen temperature interval, the thermal scan rate was calculated to be 1.5° C./min. Each sample was measured while the temperature was continuously heated until the target temperature reached 80° C. (˜40 min). The aggregation temperature (Tagg) was analyzed with onset analysis method in the DYNAMICS 7.6.0.48 software (Wyatt, Santa Barbara, Calif.).

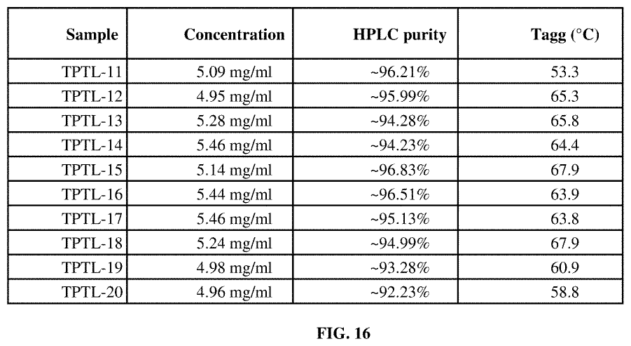

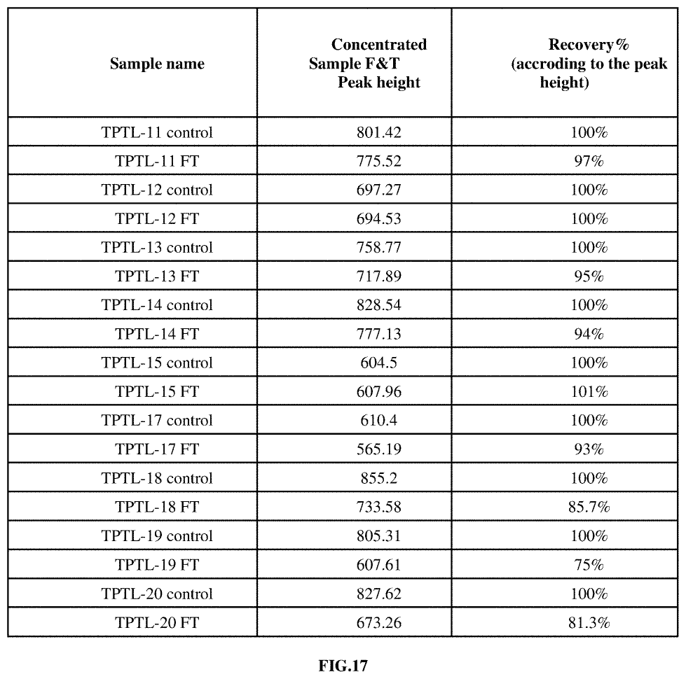

As shown in FIG. 16, Tagg of the TPTL11, TPTL19 and TPTL20 were relatively lower than the rest TABPs, which indicates these three TABPs were less stable than others.

### Freeze-Thaw Processes (5 Freeze-Thaw Cycles)

The PD-1/TIGIT/LAG-3 TABPs (TPTL11-TPTL20) were test for 5 repeated freeze-thaw cycles. Each sample was concentrated to 50 mg/ml at the special buffer (pH 6.0, 4% sucrose, 50 mM histidine, 50 mM arginine), then the sample was prepared in two parts, one froze at −80° C. as the control, the other tested with 5 Freeze-Thaw cycles; For each round of Freeze-Thaw cycle, freezing was carried out at −80° C. for at least 3 hours, then thawing at R.T. for at least 2 hours before freezing back to −80° C. again;

After 5 freeze-thaw cycles, sample was assayed with SEC-HPLC. The main peak height of the sample suffered from freeze-thaw cycles was calculated against the control one, the ratio obtained was the recovery rate. The recovery rate over 90% was thought to pass the freeze-thaw cycle criteria. As shown in FIG. 17, the recovery rate of TPTL18, TPTL19 and TPTL20 was lower than 90%. The recovery rate of TPTL11-TPTL17 was above 90%.

### Human Serum Stability

TPTL11-TPTL17 TABPs were selected for human serum stability evaluation. The antibody to be tested was prepared to a concentration of 0.5 mg/ml in 50% human serum. The solution was then aliquoted to incubate at 37° C. for 0 day, 1 Day, 7 Day and 14 Day respectively, each aliquot was stored at −20° C. when the incubation completed.

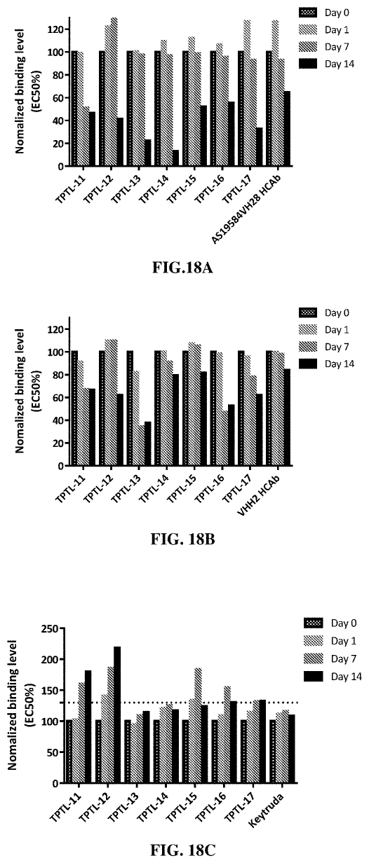

Binding activity (EC₅₀ value) of each sample was measured by Elisa when all samples were ready with 0 day sample as the 100% activity control. In this experiment, the binding activity of TABP11-TABP17 TABPs for human TIGIT, human LAG-3 and human PD-1 was determined. Antigen proteins of human TIGIT, human LAG-3 and human PD-1 were coated on the 96-well plate at 2 μg/ml overnight. After blocking, the serial concentration of TPTL11-TPTL17 TABPs was added to the coated wells. The concentration started from 5 μg/ml with 3-fold dilution. Data were analyzed with PRISM (GraphPad Software, San Diego, Calif.) using non-linear regression, and EC₅₀ values were calculated. The EC₅₀ values of all samples at Day 0 were set as 100%. The change of EC₅₀ values of Day 1, Day 7 and Day 14 were determined by (EC₅₀ day1/7/14−EC₅₀ day 0)/EC₅₀ day 0. FIG. 18A shows the binding activity of TABP11-TABP17 TABPs for human TIGIT. FIG. 18B shows the binding activity of TABP11-TABP17 TABPs for human LAG-3. FIG. 18C shows the binding activity of TABP11-TABP17 TABPs for human PD-1. As shown in the figure, TPTL-12, TPTL-15 and TPTL-16 have comparable binding for human TIGIT to positive control antibody AS19584VH28 HCAb. TPTL-14, TPTL-15 and TPTL-17 have comparable binding for human LAG-3 to positive control antibody VHH2 HCAb. TPTL-13, TPTL-14, TPTL-15, TPTL-16 and TPTL-17 have comparable binding for human PD-1 to positive control antibody Keytruda biosimilar.

All citations throughout the disclosure are hereby expressly incorporated by reference.

SEQUENCE LISTING
SEQ ID NO: 1
GGGGSGGGS
SEQ ID NO: 2
GGGGSGGGGSGGGGS
SEQ ID NO: 3
EPKSSDKTHTSPPSP
SEQ ID NO: 4
(GS)ₙ
SEQ ID NO: 5
(GSGGS)ₙ
SEQ ID NO: 6
(GGGS)ₙ
SEQ ID NO: 7
EPKSCDKTHTCPPCP
Keytruda heavy chain
SEQ ID NO: 8
QVQLVQSGVEVKKPGASVKVSCKASGYTFTNYYMYWVRQAPGQ
GLEWMGGINPSNGGTNFNEKFKNRVTLTTDSSTTTAYMELKSL
QFDDTAVYYCARRDYRFDMGFDYWGQGTTVTVSSASTKGPSVF
PLAPCSRSTSESTAALGCLVKDYFPEPVTVSWNSGALTSGVHT
FPAVLQSSGLYSLSSVVTVPSSSLGTKTYTCNVDHKPSNTKVD
KRVESKYGPPCPPCPAPEFLGGPSVFLFPPKPKDTLMISRTPE
VTCVVVDVSQEDPEVQFNWYVDGVEVHNAKTKPREEQFNSTYR
VVSVLTVLHQDWLNGKEYKCKVSNKGLPSSIEKTISKAKGQPR
EPQVYTLPPSQEEMTKNQVSLTCLVKGFYPSDIAVEWESNGQP
ENNYKTTPPVLDSDGSFFLYSRLTVDKSRWQEGNVFSCSVMHE
ALHNHYTQKSLSLSLGK
Keytruda Light chain
SEQ ID NO: 9
EIVLTQSPATLSLSPGERATLSCRASKGVSTSGYSYLHWYQQK
PGQAPRLLIYLASYLESGVPARFSGSGSGTDFTLTISSLEPED
FAVYYCQHSRDLPLTFGGGTKVEIKRTVAAPSVFIFPPSDEQL
KSGTASVVCLLNNFYPREAKVQWKVDNALQSGNSQESVTEQDS
KDSTYSLSSTLTLSKADYEKHKVYACEVTHQGLSSPVTKSFNR
GEC
Keytruda VH domain
SEQ ID NO: 10
QVQLVQSGVEVKKPGASVKVSCKASGYTFTNYYMYWVRQAPGQ
GLEWMGGINPSNGGTNFNEKFKNRVTLTTDSSTTTAYMELKSL
QFDDTAVYYCARRDYRFDMGFDYWGQGTTVTVSS
Keytruda VL domain
SEQ ID NO: 11
EIVLTQSPATLSLSPGERATLSCRASKGVSTSGYSYLHWYQQK
PGQAPRLLIYLASYLESGVPARFSGSGSGTDFTLTISSLEPED
FAVYYCQHSRDLPLTFGGGTKVEIKR
human PD-1
SEQ ID NO: 12
PGWFLDSPDRPWNPPTFSPALLVVTEGDNATFTCSFSNTSESF
VLNWYRMSPSNQTDKLAAFPEDRSQPGQDCRFRVTQLPNGRDF
HMSVVRARRNDSGTYLCGAISLAPKAQIEKESLRAELRVTERR
AEVPTAHPSPSPRPAGQFQTLVVGVVGGLLGSLVLLVWVLAVI
CSRAARGTIGARRTGQPLKEDPSAVPVFSVDYGELDFQWREKT
PEPPVPCVPEQTEYATIVFPSGMGTSSPARRGSADGPRSAQPL
RPEDGHCSWPL
human TIGIT
SEQ ID NO: 13
MMTGTIETTGNISAEKGGSIILQCHLSSTTAQVTQVNWEQQDQ
LLAICNADLGWHISPSFKDRVAPGPGLGLTLQSLTVNDTGEYF
CIYHTYPDGTYTGRIFLEVLESSVAEHGARFQIPLLGAMAATL
VVICTAVIVVVALTRKKKALRIHSVEGDLRRKSAGQEEWSPSA
PSPPGSCVQAEAAPAGLCGEQRGEDCAELHDYFNVLSYRSLGN
CSFFTETG
human LAG-3
SEQ ID NO: 14
VPVVWAQEGAPAQLPCSPTIPLQDLSLLRRAGVTWQHQPDSGP
PAAAPGHPLAPGPHPAAPSSWGPRPRRYTVLSVGPGGLRSGRL
PLQPRVQLDERGRQRGDFSLWLRPARRADAGEYRAAVHLRDRA
LSCRLRLRLGQASMTASPPGSLRASDWVILNCSFSRPDRPASV
HWFRNRGQGRVPVRESPHHHLAESFLFLPQVSPMDSGPWGCIL
TYRDGFNVSIMYNLTVLGLEPPTPLTVYAGAGSRVGLPCRLPA
GVGTRSFLTAKWTPPGGGPDLLVTGDNGDFTLRLEDVSQAQAG
TYTCHIHLQEQQLNATVTLAIITVTPKSFGSPGSLGKLLCEVT
PVSGQERFVWSSLDTPSQRSFSGPWLEAQEAQLLSQPWQCQLY
QGERLLGAAVYFTELSSPGAQRSGRAPGALPAGHLLLFLILGV
LSLLLLVTGAFGFHLWRRQWRPRRFSALEQGIHPPQAQSKIEE
LEQEPEPEPEPEPEPEPEPEPEQL

TABLE 3
Exemplary of PD-1/TIGIT/LAG-3 trispecific antigen binding proteins (TABPs) amino
acid sequences (linker sequence is bolded)

| | |
|---|---|
| SEQ ID NO: 15 | EVQLVESGGGLVQPGGSLRLSCAASGYTVSSYCMGWFRQAPGKGREGVSAIDSDGSVSYADSVKGRFTISKDNSKNTLYL |
| TPTL-11 Light | QMNSLRAEDTAVYFCAADLCWVDQDQGEYNTWGQGTLVTVSSEPKSSDKTHTSPPSPEIVLTQSPATLSLSPGERATLSC |
| chain | RASKGVSTSGYSYLHWYQQKPGQAPRLLIYLASYLESGVPARFSGSGSGTDFTLTISSLEPEDFAVYYCQHSRDLPLTFGGGT |
| | KVEIKRTVAAPSVFIFPPSDEQLKSGTASVVCLLNNFYPREAKVQWKVDNALQSGNSQESVTEQDSKDSTYSLSSTLTLSKA |
| | DYEKHKVYACEVTHQGLSSPVTKSFNRGEC |
| SEQ ID NO: 16 | EVQLVESGGGLVQPGGSLRLSCAASGYKYGVYSMGWFRQAPGKGLEGVSAICSGGRTTYSDSVKGRFTISRDNSNQILYLQ |
| TPTL-11 Heavy | MNSLRAEDTAVYYCAARPLWTGDCDLSSSWYKTWGQGTLVTVSSEPKSSDKTHTSPPSPQVQLVQSGVEVKKPGASVKV |
| chain | SCKASGYTFTNYYMYWVRQAPGQGLEWMGGINPSNGGTNFNEKFKNRVTLTTDSSTTTAYMELKSLQFDDTAVYYCAR |
| | RDYRFDMGFDYWGQGTTVTVSSASTKGPSVFPLAPCSRSTSESTAALGCLVKDYFPEPVTVSWNSGALTSGVHTFPAVLQS |
| | SGLYSLSSVVTVPSSSLGTKTYTCNVDHKPSNTKVDKRVESKYGPPCPPCPAPEFLGGPSVFLFPPKPKDTLMISRTPEVTCV |
| | VVDVSQEDPEVQFNWYVDGVEVHNAKTKPREEQFNSTYRVVSVLTVLHQDWLNGKEYKCKVSNKGLPSSIEKTISKAKG |
| | QPREPQVYTLPPSQEEMTKNQVSLTCLVKGFYPSDIAVEWESNGQPENNYKTTPPVLDSDGSFFLYSRLTVDKSRWQEGN |
| | VFSCSVMHEALHNHYTQKSLSLSLGK |
| SEQ ID NO: 17 | EIVLTQSPATLSLSPGERATLSCRASKGVSTSGYSYLHWYQQKPGQAPRLLIYLASYLESGVPARFSGSGSGTDFTLTISSLEPE |
| TPTL-12 Light | DFAVYYCQHSRDLPLTFGGGTKVEIKRTVAAPSVFIFPPSDEQLKSGTASVVCLLNNFYPREAKVQWKVDNALQSGNSQES |
| chain | VTEQDSKDSTYSLSSTLTLSKADYEKHKVYACEVTHQGLSSPVTKSFNRGEC |
| SEQ ID NO: 18 | EVQLVESGGGLVQPGGSLRLSCAASGYKYGVYSMGWFRQAPGKGLEGVSAICSGGRTTYSDSVKGRFTISRDNSNQILYLQ |
| TPTL-12 Heavy | MNSLRAEDTAVYYCAARPLWTGDCDLSSSWYKTWGQGTLVTVSSEPKSSDKTHTSPPSPEVQLVESGGGLVQPGGSLRL |
| chain | SCAASGYTVSSYCMGWFRQAPGKGREGVSAIDSDGSVSYADSVKGRFTISKDNSKNTLYLQMNSLRAEDTAVYFCAADLC |
| | WVDQDQGEYNTWGQGTLVTVSSEPKSSDKTHTSPPSPQVQLVQSGVEVKKPGASVKVSCKASGYTFTNYYMYWVRQA |
| | PGQGLEWMGGINPSNGGTNFNEKFKNRVTLTTDSSTTTAYMELKSLQFDDTAVYYCARRDYRFDMGFDYWGQGTTVTV |
| | SSASTKGPSVFPLAPCSRSTSESTAALGCLVKDYFPEPVTVSWNSGALTSGVHTFPAVLQSSGLYSLSSVVTVPSSSLGTKTYT |
| | CNVDHKPSNTKVDKRVESKYGPPCPPCPAPEFLGGPSVFLFPPKPKDTLMISRTPEVTCVVVDVSQEDPEVQFNWYVDGV |
| | EVHNAKTKPREEQFNSTYRVVSVLTVLHQDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKSRWQEGNVFSCSVMHEALHNHYTQKSLSL |
| | SLGK |
| SEQ ID NO: 19 | EVQLVESGGGLVQPGGSLRLSCAASGYKYGVYSMGWFRQAPGKGLEGVSAICSGGRTTYSDSVKGRFTISRDNSNQILYLQ |
| TPTL-13 Light | MNSLRAEDTAVYYCAARPLWTGDCDLSSSWYKTWGQGTLVTVSSEPKSSDKTHTSPPSPEVQLVESGGGLVQPGGSLRL |
| chain | SCAASGYTVSSYCMGWFRQAPGKGREGVSAIDSDGSVSYADSVKGRFTISKDNSKNTLYLQMNSLRAEDTAVYFCAADLC |
| | WVDQDQGEYNTWGQGTLVTVSSEPKSSDKTHTSPPSPEIVLTQSPATLSLSPGERATLSCRASKGVSTSGYSYLHWYQQK |
| | PGQAPRLLIYLASYLESGVPARFSGSGSGTDFTLTISSLEPEDFAVYYCQHSRDLPLTFGGGTKVEIKRTVAAPSVFIFPPSDEQ |
| | LKSGTASVVCLLNNFYPREAKVQWKVDNALQSGNSQESVTEQDSKDSTYSLSSTLTLSKADYEKHKVYACEVTHQGLSSPV |
| | TKSFNRGEC |
| SEQ ID NO: 20 | QVQLVQSGVEVKKPGASVKVSCKASGYTFTNYYMYWVRQAPGQGLEWMGGINPSNGGTNFNEKFKNRVTLTTDSSTT |
| TPTL-13 Heavy | TAYMELKSLQFDDTAVYYCARRDYRFDMGFDYWGQGTTVTVSSASTKGPSVFPLAPCSRSTSESTAALGCLVKDYFPEPVT |
| chain | VSWNSGALTSGVHTFPAVLQSSGLYSLSSVVTVPSSSLGTKTYTCNVDHKPSNTKVDKRVESKYGPPCPPCPAPEFLGGPSV |
| | FLFPPKPKDTLMISRTPEVTCVVVDVSQEDPEVQFNWYVDGVEVHNAKTKPREEQFNSTYRVVSVLTVLHQDWLNGKEY |
| | KCKVSNKGLPSSIEKTISKAKGQPREPQVYTLPPSQEEMTKNQVSLTCLVKGFYPSDIAVEWESNGQPENNYKTTPPVLDSD |
| | GSFFLYSRLTVDKSRWQEGNVFSCSVMHEALHNHYTQKSLSLSLGK |
| SEQ ID NO: 21 | EVQLVESGGGLVQPGGSLRLSCAASGYKYGVYSMGWFRQAPGKGLEGVSAICSGGRTTYSDSVKGRFTISRDNSNQILYLQ |
| TPTL-14 Light | MNSLRAEDTAVYYCAARPLWTGDCDLSSSWYKTWGQGTLVTVSSEPKSSDKTHTSPPSPEIVLTQSPATLSLSPGERATLSC |
| chain | RASKGVSTSGYSYLHWYQQKPGQAPRLLIYLASYLESGVPARFSGSGSGTDFTLTISSLEPEDFAVYYCQHSRDLPLTFGGGT |
| | KVEIKRTVAAPSVFIFPPSDEQLKSGTASVVCLLNNFYPREAKVQWKVDNALQSGNSQESVTEQDSKDSTYSLSSTLTLSKA |
| | DYEKHKVYACEVTHQGLSSPVTKSFNRGECEPKSSDKTHTSPPSPEVQLVESGGGLVQPGGSLRLSCAASGYTVSSYCMG |
| | WFRQAPGKGREGVSAIDSDGSVSYADSVKGRFTISKDNSKNTLYLQMNSLRAEDTAVYFCAADLCWVDQDQGEYNTWG |
| | QGTLVTVSS |
| SEQ ID NO: 20 | The sequence is the same as TPTL-13 Heavy chain. |
| TPTL-14 Heavy | |
| chain | |
| SEQ ID NO: 22 | EIVLTQSPATLSLSPGERATLSCRASKGVSTSGYSYLHWYQQKPGQAPRLLIYLASYLESGVPARFSGSGSGTDFTLTISSLEPE |
| TPTL-15 Light | DFAVYYCQHSRDLPLTFGGGTKVEIKRTVAAPSVFIFPPSDEQLKSGTASVVCLLNNFYPREAKVQWKVDNALQSGNSQES |
| chain | VTEQDSKDSTYSLSSTLTLSKADYEKHKVYACEVTHQGLSSPVTKSFNRGECEPKSSDKTHTSPPSPEVQLVESGGGLVQPG |
| | GSLRLSCAASGYTVSSYCMGWFRQAPGKGREGVSAIDSDGSVSYADSVKGRFTISKDNSKNTLYLQMNSLRAEDTAVYFCA |
| | ADLCWVDQDQGEYNTWGQGTLVTVSS |
| SEQ ID NO: 16 | The sequence is the same as TPTL-11 Heavy chain. |
| TPTL-15 Heavy | |
| chain | |
| SEQ ID NO: 17 | The sequence is the same as TPTL-12 Light chain. |
| TPTL-16 Light | |
| chain | |
| SEQ ID NO: 23 | EVQLVESGGGLVQPGGSLRLSCAASGYKYGVYSMGWFRQAPGKGLEGVSAICSGGRTTYSDSVKGRFTISRDNSNQILYLQ |
| TPTL-16 Heavy | MNSLRAEDTAVYYCAARPLWTGDCDLSSSWYKTWGQGTLVTVSSEPKSSDKTHTSPPSPQVQLVQSGVEVKKPGASVKV |
| chain | SCKASGYTFTNYYMYWVRQAPGQGLEWMGGINPSNGGTNFNEKFKNRVTLTTDSSTTTAYMELKSLQFDDTAVYYCAR |
| | RDYRFDMGFDYWGQGTTVTVSSASTKGPSVFPLAPCSRSTSESTAALGCLVKDYFPEPVTVSWNSGALTSGVHTFPAVLQS |
| | SGLYSLSSVVTVPSSSLGTKTYTCNVDHKPSNTKVDKRVESKYGPPCPPCPAPEFLGGPSVFLFPPKPKDTLMISRTPEVTCV |
| | VVDVSQEDPEVQFNWYVDGVEVHNAKTKPREEQFNSTYRVVSVLTVLHQDWLNGKEYKCKVSNKGLPSSIEKTISKAKG |
| | QPREPQVYTLPPSQEEMTKNQVSLTCLVKGFYPSDIAVEWESNGQPENNYKTTPPVLDSDGSFFLYSRLTVDKSRWQEGN |
| | VFSCSVMHEALHNHYTQKSLSLSLGKEPKSSDKTHTSPPSPEVQLVESGGGLVQPGGSLRLSCAASGYTVSSYCMGWFRQ |
| | APGKGREGVSAIDSDGSVSYADSVKGRFTISKDNSKNTLYLQMNSLRAEDTAVYFCAADLCWVDQDQGEYNTWGQGTLV |
| | TVSS |
| SEQ ID NO: 24 | EVQLVESGGGLVQPGGSLRLSCAASGYKYGVYSMGWFRQAPGKGLEGVSAICSGGRTTYSDSVKGRFTISRDNSNQILYLQ |
| TPTL-17 Light | MNSLRAEDTAVYYCAARPLWTGDCDLSSSWYKTWGQGTLVTVSSEPKSSDKTHTSPPSPEIVLTQSPATLSLSPGERATLSC |
| chain | RASKGVSTSGYSYLHWYQQKPGQAPRLLIYLASYLESGVPARFSGSGSGTDFTLTISSLEPEDFAVYYCQHSRDLPLTFGGGT |
| | KVEIKRTVAAPSVFIFPPSDEQLKSGTASVVCLLNNFYPREAKVQWKVDNALQSGNSQESVTEQDSKDSTYSLSSTLTLSKA |
| | DYEKHKVYACEVTHQGLSSPVTKSFNRGEC |
| SEQ ID NO: 25 | QVQLVQSGVEVKKPGASVKVSCKASGYTFTNYYMYWVRQAPGQGLEWMGGINPSNGGTNFNEKFKNRVTLTTDSSTT |
| TPTL-17 Heavy | TAYMELKSLQFDDTAVYYCARRDYRFDMGFDYWGQGTTVTVSSASTKGPSVFPLAPCSRSTSESTAALGCLVKDYFPEPVT |
| chain | VSWNSGALTSGVHTFPAVLQSSGLYSLSSVVTVPSSSLGTKTYTCNVDHKPSNTKVDKRVESKYGPPCPPCPAPEFLGGPSV |
| | FLFPPKPKDTLMISRTPEVTCVVVDVSQEDPEVQFNWYVDGVEVHNAKTKPREEQFNSTYRVVSVLTVLHQDWLNGKEY |
| | KCKVSNKGLPSSIEKTISKAKGQPREPQVYTLPPSQEEMTKNQVSLTCLVKGFYPSDIAVEWESNGQPENNYKTTPPVLDSD |
| | GSFFLYSRLTVDKSRWQEGNVFSCSVMHEALHNHYTQKSLSLSLGKEPKSSDKTHTSPPSPEVQLVESGGGLVQPGGSLRL |
| | SCAASGYTVSSYCMGWFRQAPGKGREGVSAIDSDGSVSYADSVKGRFTISKDNSKNTLYLQMNSLRAEDTAVYFCAADLC |
| | WVDQDQGEYNTWGQGTLVTVSS |
| SEQ ID NO: 22 | The sequence is the same as TPTL-15 Light chain. |
| TPTL-18 Light | |
| chain | |
| SEQ ID NO: 26 | QVQLVQSGVEVKKPGASVKVSCKASGYTFTNYYMYWVRQAPGQGLEWMGGINPSNGGTNFNEKFKNRVTLTTDSSTT |
| TPTL-18 Heavy | TAYMELKSLQFDDTAVYYCARRDYRFDMGFDYWGQGTTVTVSSASTKGPSVFPLAPCSRSTSESTAALGCLVKDYFPEPVT |
| chain | VSWNSGALTSGVHTFPAVLQSSGLYSLSSVVTVPSSSLGTKTYTCNVDHKPSNTKVDKRVESKYGPPCPPCPAPEFLGGPSV |
| | FLFPPKPKDTLMISRTPEVTCVVVDVSQEDPEVQFNWYVDGVEVHNAKTKPREEQFNSTYRVVSVLTVLHQDWLNGKEY |
| | KCKVSNKGLPSSIEKTISKAKGQPREPQVYTLPPSQEEMTKNQVSLTCLVKGFYPSDIAVEWESNGQPENNYKTTPPVLDSD |
| | GSFFLYSRLTVDKSRWQEGNVFSCSVMHEALHNHYTQKSLSLSLGKEPKSSDKTHTSPPSPEVQLVESGGGLVQPGGSLRL |
| | SCAASGYKYGVYSMGWFRQAPGKGLEGVSAICSGGRTTYSDSVKGRFTISRDNSNQILYLQMNSLRAEDTAVYYCAARPL |
| | WTGDCDLSSSWYKTWGQGTLVTVSS |
| SEQ ID NO: 27 | EIVLTQSPATLSLSPGERATLSCRASKGVSTSGYSYLHWYQQKPGQAPRLLIYLASYLESGVPARFSGSGSGTDFTLTISSLEPE |
| TPTL-19 Light | DFAVYYCQHSRDLPLTFGGGTKVEIKRTVAAPSVFIFPPSDEQLKSGTASVVCLLNNFYPREAKVQWKVDNALQSGNSQES |
| chain | VTEQDSKDSTYSLSSTLTLSKADYEKHKVYACEVTHQGLSSPVTKSFNRGECEPKSSDKTHTSPPSPEVQLVESGGGLVQPG |
| | GSLRLSCAASGYTVSSYCMGWFRQAPGKGREGVSAIDSDGSVSYADSVKGRFTISKDNSKNTLYLQMNSLRAEDTAVYFCA |
| | ADLCWVDQDQGEYNTWGQGTLVTVSSEPKSSDKTHTSPPSPEVQLVESGGGLVQPGGSLRLSCAASGYKYGVYSMGW |
| | FRQAPGKGLEGVSAICSGGRTTYSDSVKGRFTISRDNSNQILYLQMNSLRAEDTAVYYCAARPLWTGDCDLSSSWYKTWG |
| | QGTLVTVSS |
| SEQ ID NO: 20 | The sequence is the same as TPTL-13 Heavy chain. |
| TPTL-19 Heavy | |
| chain | |
| SEQ ID NO: 17 | The sequence is the same as TPTL-12 Light chain. |
| TPTL-20 Light | |
| chain | |
| SEQ ID NO: 28 | QVQLVQSGVEVKKPGASVKVSCKASGYTFTNYYMYWVRQAPGQGLEWMGGINPSNGGTNFNEKFKNRVTLTTDSSTT |
| TPTL-20 Heavy | TAYMELKSLQFDDTAVYYCARRDYRFDMGFDYWGQGTTVTVSSASTKGPSVFPLAPCSRSTSESTAALGCLVKDYFPEPVT |
| chain | VSWNSGALTSGVHTFPAVLQSSGLYSLSSVVTVPSSSLGTKTYTCNVDHKPSNTKVDKRVESKYGPPCPPCPAPEFLGGPSV |
| | FLFPPKPKDTLMISRTPEVTCVVVDVSQEDPEVQFNWYVDGVEVHNAKTKPREEQFNSTYRVVSVLTVLHQDWLNGKEY |
| | KCKVSNKGLPSSIEKTISKAKGQPREPQVYTLPPSQEEMTKNQVSLTCLVKGFYPSDIAVEWESNGQPENNYKTTPPVLDSD |
| | GSFFLYSRLTVDKSRWQEGNVFSCSVMHEALHNHYTQKSLSLSLGKEPKSSDKTHTSPPSPEVQLVESGGGLVQPGGSLRL |
| | SCAASGYTVSSYCMGWFRQAPGKGREGVSAIDSDGSVSYADSVKGRFTISKDNSKNTLYLQMNSLRAEDTAVYFCAADLC |
| | WVDQDQGEYNTWGQGTLVTVSSEPKSSDKTHTSPPSPEVQLVESGGGLVQPGGSLRLSCAASGYKYGVYSMGWFRQA |
| | PGKGLEGVSAICSGGRTTYSDSVKGRFTISRDNSNQILYLQMNSLRAEDTAVYYCAARPLWTGDCDLSSSWYKTWGQGTL |
| | VTVSS |

AS19584VH28 HCAb
SEQ ID NO: 29
EVQLVESGGGLVQPGGSLRLSCAASGYKYGVYSMGWFRQAPGKGLEGVSA
ICSGGRTTYSDSVKGRFTISRDNSNQILYLQMNSLRAEDTAVYYCAARPL
WTGDCDLSSSWYKTWGQGTLVTVSSESKYGPPCPPCPAPEFLGGPSVFLF
PPKPKDTLMISRTPEVTCVVVDVSQEDPEVQFNVVYVDGVEVHNAKTKPR
EEQFNSTYRVVSVLTVLHQDWLNGKEYKCKVSNKGLPSSIEKTISKAKGQ
PREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLS
LSLGK
VHH2 HCAb
SEQ ID NO: 30
EVQLVESGGGLVQPGGSLRLSCAASGYTVSSYCMGWFRQAPGKGREGVSA
EDSDGSVSYADSVKGRFTISKDNSKNTLYLQMNSLRAEDTAVYFCAADLC
WVDQDQGEYNTWGQGTLVTVSSESKYGPPCPPCPAPEFLGGPSVFLFPPK
PKDTLMISRTPEVTCVVVDVSQEDPEVQFNVVYVDGVEVHNAKTKPREEQ
FNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSL
GK
AS19584VH28 sdAb
SEQ ID NO: 31
EVQLVESGGGLVQPGGSLRLSCAASGYKYGVYSMGWFRQAPGKGLEGVSA
ICSGGRTTYSDSVKGRFTISRDNSNQILYLQMNSLRAEDTAVYYCAARPL
WTGDCDLSSSWYKTWGQGTLVTVSS
VHH2 sdAb
SEQ ID NO: 32
EVQLVESGGGLVQPGGSLRLSCAASGYTVSSYCMGWFRQAPGKGREGVSA
EDSDGSVSYADSVKGRFTISKDNSKNTLYLQMNSLRAEDTAVYFCAADLC
WVDQDQGEYNTWGQGTLVTVSS

1. A multispecific antigen binding protein (MABP) comprising:
(a) a first antigen binding portion comprising a heavy chain variable domain (V_{H}) and a light chain variable domain (V_{L}), wherein the V_{H} and V_{L} together form an antigen-binding site that specifically binds a first epitope,
(b) a second antigen binding portion comprising a first single-domain antibody (sdAb) that specifically binds a second epitope, and
(c) a third antigen binding portion comprising a second single-domain antibody (sdAb) that specifically binds a third epitope,
wherein the first antigen binding portion, the second antigen binding portion, and the third antigen binding portion are fused to each other.

2-4. (canceled)

5. The MABP of claim 1, wherein the first sdAb and/or the second sdAb is a V_{H}H.

6. The MABP of claim 5, wherein the MABP comprises:
(i)
(1) a first polypeptide comprising from the N-terminus to the C-terminus: the first sdAb and a heavy chain of the first antigen binding portion; and
(2) a second polypeptide comprising from the N-terminus to the C-terminus: the second sdAb and a light chain of the first antigen binding portion;
(ii)
(1) a first polypeptide comprising from the N-terminus to the C-terminus: a heavy chain of the first antigen binding portion and the first sdAb; and
(2) a second polypeptide comprising from the N-terminus to the C-terminus: the second sdAb and a light chain of the first antigen binding portion;
(iii)
(1) a first polypeptide comprising from the N-terminus to the C-terminus: the first sdAb and a heavy chain of the first antigen binding portion; and
(2) a second polypeptide comprising from the N-terminus to the C-terminus: a light chain of the first antigen binding portion and the second sdAb;
(iv)
(1) a first polypeptide comprising from the N-terminus to the C-terminus: a heavy chain of the first antigen binding portion and the first sdAb; and
(2) a second polypeptide comprising from the N-terminus to the C-terminus: a light chain of the first antigen binding portion and the second sdAb;
(v)
(1) a first polypeptide comprising from the N-terminus to the C-terminus: the first sdAb, a heavy chain of the first antigen binding portion, and the second sdAb; and
(2) a second polypeptide comprising from the N-terminus to the C-terminus: a light chain of the first antigen binding portion; or
(vi)
(1) a first polypeptide comprising from the N-terminus to the C-terminus: a heavy chain of the first antigen binding portion; and
(2) a second polypeptide comprising from the N-terminus to the C-terminus: the first sdAb, a light chain of the first antigen binding portion and the second sdAb;
(vii)
(1) a first polypeptide comprising from the N-terminus to the C-terminus: the first sdAb, the second sdAb, and a heavy chain of the first antigen binding portion; and
(2) a second polypeptide comprising from the N-terminus to the C-terminus: a light chain of the first antigen binding portion;
(viii)
(1) a first polypeptide comprising from the N-terminus to the C-terminus: a heavy chain of the first antigen binding portion, the first sdAb, and the second sdAb; and
(2) a second polypeptide comprising from the N-terminus to the C-terminus: a light chain of the first antigen binding portion;
(ix)
(1) a first polypeptide comprising from the N-terminus to the C-terminus: a heavy chain of the first antigen binding portion; and
(2) a second polypeptide comprising from the N-terminus to the C-terminus: the first sdAb, the second sdAb, and a light chain of the first antigen binding portion; or
(x)
(1) a first polypeptide comprising from the N-terminus to the C-terminus: a heavy chain of the first antigen binding portion; and
(2) a second polypeptide comprising from the N-terminus to the C-terminus: a light chain of the first antigen binding portion, the first sdAb, and the second sdAb.

7-15. (canceled)

16. The MABP of claim 6, wherein the MABP comprises two chains of the first polypeptide and two chains of the second polypeptide.

17. The MABP of claim 1, wherein the first epitope, the second epitope and/or the third epitope is from an immune checkpoint molecule.

18. The MABP of claim 17, wherein the immune checkpoint molecule is selected from the group consisting of PD-1, PD-L1, PD-L2, CTLA-4, B7-H3, TIM-3, LAG-3, TIGIT, VISTA, ICOS, 4-1BB, OX40, GITR, and CD40.

19. The MABP of claim 18, wherein the first antigen binding portion is an anti-PD-1 antibody or antigen binding fragment thereof.

20. The MABP of claim 19, wherein the anti-PD-1 antibody is derived from pembrolizumab.

21. The MABP of claim 17, wherein the second antigen binding portion comprises an anti-TIGIT sdAb.

22. The MABP of claim 21, wherein the anti-TIGIT sdAb comprises the amino acid sequence of SEQ ID NO: 31, or a variant thereof comprising up to about 3 amino acid substitutions.

23. The MABP of claim 17, wherein the third antigen binding portion comprises an anti-LAG-3 sdAb.

24. The MABP of claim 23, wherein the anti-LAG-3 sdAb comprises the amino acid sequence of SEQ ID NO: 32, or a variant thereof comprising up to about 3 amino acid substitutions.

25. The MABP of claim 17, wherein the MABP comprises:
(1) a first polypeptide comprising the amino acid sequence of any one of SEQ ID NOs: 16, 18, 20, 23, 25, 26, 28, or a variant thereof comprising up to about 5 amino acid substitutions; and
(2) a second polypeptide comprising the amino acid sequence of any one of SEQ ID NOs:15, 17, 19, 21, 22, 24, 27, or a variant thereof comprising up to about 5 amino acid substitutions.

26. The MABP of claim 1, wherein the first epitope, the second epitope and/or the third epitope is from a tumor antigen, a cell surface antigen of an immune effector cell, a pro-inflammatory molecule, or an angiogenic factor.

27-43. (canceled)

44. The MABP of claim 1, wherein the first antigen binding portion comprises an Fc region.

45-46. (canceled)

47. The MABP of claim 1, wherein the first antigen binding portion, the second antigen binding portion and/or the third antigen binding portion are fused to each other via a peptide linker.

48. The MABP of claim 47, wherein the peptide linker is no more than about 30 amino acids long.

49-50. (canceled)

51. The MABP of claim 1, wherein:
(i) the multispecific antigen binding protein has an aggregation onset temperature of at least about 55° C.;
(ii) the multispecific antigen binding protein is stable for at least about one week at 25° C. at a concentration of at least about 50 mg/mL; and/or
(iii) the multispecific antigen binding protein is stable after at least about 5 freeze-thaw cycles at a concentration of at least 50 mg/mL.

52-53. (canceled)

54. A pharmaceutical composition comprising the MABP of claim 1 and a pharmaceutically acceptable carrier.

55. A method of treating a disease in an individual, comprising administering to the individual an effective amount of the pharmaceutical composition of claim 54.

56-59. (canceled)


SEQUENCE LISTING (32 sequences)

SEQ ID NO: 1 - protein, 9 aa, Artificial Sequence
  linker
GGGGSGGGS

SEQ ID NO: 2 - protein, 15 aa, Artificial Sequence
  linker
GGGGSGGGGSGGGGS

SEQ ID NO: 3 - protein, 15 aa, Artificial Sequence
  linker
EPKSSDKTHTSPPSP

SEQ ID NO: 4 - protein, 2 aa, Artificial Sequence
  linker
  VARIANT (1)..(2): Can be present in repeats of one or more
GS

SEQ ID NO: 5 - protein, 5 aa, Artificial Sequence
  linker
  VARIANT (1)..(5): Can be present in repeats of one or more
GSGGS

SEQ ID NO: 6 - protein, 4 aa, Artificial Sequence
  linker
  VARIANT (1)..(4): Can be present in repeats of one or more
GGGS

SEQ ID NO: 7 - protein, 15 aa, Artificial Sequence
  linker
EPKSCDKTHTCPPCP

SEQ ID NO: 8 - protein, 447 aa, Artificial Sequence
  Keytruda heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 218 aa, Artificial Sequence
  Keytruda Light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 120 aa, Artificial Sequence [sequence omitted]

SEQ ID NO: 11 - protein, 112 aa, Artificial Sequence
  Keytruda VL domain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 12 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 13 - protein, 223 aa, Homo sapiens [sequence omitted]

SEQ ID NO: 14 - protein, 497 aa, Homo sapiens
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 15 - protein, 355 aa, Artificial Sequence
  TPTL-11 Light chain
EVQLVESGGGLVQPGGSLRLSCAASGYTVSSYCMGWFRQAPGKGREGVSAIDSDGSVSYADSVKGRFTISKDNSKNTLYLQMNSLRAEDTAVYFCAADLCWVDQDQGEYNTWGQGTLVTVSSEPKSSDKTHTSPPSPEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 587 aa, Artificial Sequence
  TPTL-11 Heavy chain
EVQLVESGGGLVQPGGSLRLSCAASGYKYGVYSMGWFRQAPGKGLEGVSAICSGGRTTYSDSVKGRFTISRDNSNQILYLQMNSLRAEDTAVYYCAARPLWTGDCDLSSSWYKTWGQGTLVTVSSEPKSSDKTHTSPPSPQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 218 aa, Artificial Sequence; identical to SEQ ID NO: 9
  TPTL-12 Light chain

SEQ ID NO: 18 - protein, 724 aa, Artificial Sequence
  TPTL-12 Heavy chain
EVQLVESGGGLVQPGGSLRLSCAASGYKYGVYSMGWFRQAPGKGLEGVSAICSGGRTTYSDSVKGRFTISRDNSNQILYLQMNSLRAEDTAVYYCAARPLWTGDCDLSSSWYKTWGQGTLVTVSSEPKSSDKTHTSPPSPEVQLVESGGGLVQPGGSLRLSCAASGYTVSSYCMGWFRQAPGKGREGVSAIDSDGSVSYADSVKGRFTISKDNSKNTLYLQMNSLRAEDTAVYFCAADLCWVDQDQGEYNTWGQGTLVTVSSEPKSSDKTHTSPPSPQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 495 aa, Artificial Sequence
  TPTL-13 Light chain
EVQLVESGGGLVQPGGSLRLSCAASGYKYGVYSMGWFRQAPGKGLEGVSAICSGGRTTYSDSVKGRFTISRDNSNQILYLQMNSLRAEDTAVYYCAARPLWTGDCDLSSSWYKTWGQGTLVTVSSEPKSSDKTHTSPPSPEVQLVESGGGLVQPGGSLRLSCAASGYTVSSYCMGWFRQAPGKGREGVSAIDSDGSVSYADSVKGRFTISKDNSKNTLYLQMNSLRAEDTAVYFCAADLCWVDQDQGEYNTWGQGTLVTVSSEPKSSDKTHTSPPSPEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 447 aa, Artificial Sequence; identical to SEQ ID NO: 8
  TPTL-13 Heavy chain

SEQ ID NO: 21 - protein, 495 aa, Artificial Sequence
  TPTL-14 Light chain
EVQLVESGGGLVQPGGSLRLSCAASGYKYGVYSMGWFRQAPGKGLEGVSAICSGGRTTYSDSVKGRFTISRDNSNQILYLQMNSLRAEDTAVYYCAARPLWTGDCDLSSSWYKTWGQGTLVTVSSEPKSSDKTHTSPPSPEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSSDKTHTSPPSPEVQLVESGGGLVQPGGSLRLSCAASGYTVSSYCMGWFRQAPGKGREGVSAIDSDGSVSYADSVKGRFTISKDNSKNTLYLQMNSLRAEDTAVYFCAADLCWVDQDQGEYNTWGQGTLVTVSS

SEQ ID NO: 22 - protein, 355 aa, Artificial Sequence
  TPTL-15 Light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSSDKTHTSPPSPEVQLVESGGGLVQPGGSLRLSCAASGYTVSSYCMGWFRQAPGKGREGVSAIDSDGSVSYADSVKGRFTISKDNSKNTLYLQMNSLRAEDTAVYFCAADLCWVDQDQGEYNTWGQGTLVTVSS

SEQ ID NO: 23 - protein, 724 aa, Artificial Sequence
  TPTL-16 Heavy chain
EVQLVESGGGLVQPGGSLRLSCAASGYKYGVYSMGWFRQAPGKGLEGVSAICSGGRTTYSDSVKGRFTISRDNSNQILYLQMNSLRAEDTAVYYCAARPLWTGDCDLSSSWYKTWGQGTLVTVSSEPKSSDKTHTSPPSPQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKEPKSSDKTHTSPPSPEVQLVESGGGLVQPGGSLRLSCAASGYTVSSYCMGWFRQAPGKGREGVSAIDSDGSVSYADSVKGRFTISKDNSKNTLYLQMNSLRAEDTAVYFCAADLCWVDQDQGEYNTWGQGTLVTVSS

SEQ ID NO: 24 - protein, 358 aa, Artificial Sequence
  TPTL-17 Light chain
EVQLVESGGGLVQPGGSLRLSCAASGYKYGVYSMGWFRQAPGKGLEGVSAICSGGRTTYSDSVKGRFTISRDNSNQILYLQMNSLRAEDTAVYYCAARPLWTGDCDLSSSWYKTWGQGTLVTVSSEPKSSDKTHTSPPSPEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 584 aa, Artificial Sequence
  TPTL-17 Heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKEPKSSDKTHTSPPSPEVQLVESGGGLVQPGGSLRLSCAASGYTVSSYCMGWFRQAPGKGREGVSAIDSDGSVSYADSVKGRFTISKDNSKNTLYLQMNSLRAEDTAVYFCAADLCWVDQDQGEYNTWGQGTLVTVSS

SEQ ID NO: 26 - protein, 587 aa, Artificial Sequence
  TPTL-18 Heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKEPKSSDKTHTSPPSPEVQLVESGGGLVQPGGSLRLSCAASGYKYGVYSMGWFRQAPGKGLEGVSAICSGGRTTYSDSVKGRFTISRDNSNQILYLQMNSLRAEDTAVYYCAARPLWTGDCDLSSSWYKTWGQGTLVTVSS

SEQ ID NO: 27 - protein, 495 aa, Artificial Sequence
  TPTL-19 Light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSSDKTHTSPPSPEVQLVESGGGLVQPGGSLRLSCAASGYTVSSYCMGWFRQAPGKGREGVSAIDSDGSVSYADSVKGRFTISKDNSKNTLYLQMNSLRAEDTAVYFCAADLCWVDQDQGEYNTWGQGTLVTVSSEPKSSDKTHTSPPSPEVQLVESGGGLVQPGGSLRLSCAASGYKYGVYSMGWFRQAPGKGLEGVSAICSGGRTTYSDSVKGRFTISRDNSNQILYLQMNSLRAEDTAVYYCAARPLWTGDCDLSSSWYKTWGQGTLVTVSS

SEQ ID NO: 28 - protein, 724 aa, Artificial Sequence
  TPTL-20 Heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKEPKSSDKTHTSPPSPEVQLVESGGGLVQPGGSLRLSCAASGYTVSSYCMGWFRQAPGKGREGVSAIDSDGSVSYADSVKGRFTISKDNSKNTLYLQMNSLRAEDTAVYFCAADLCWVDQDQGEYNTWGQGTLVTVSSEPKSSDKTHTSPPSPEVQLVESGGGLVQPGGSLRLSCAASGYKYGVYSMGWFRQAPGKGLEGVSAICSGGRTTYSDSVKGRFTISRDNSNQILYLQMNSLRAEDTAVYYCAARPLWTGDCDLSSSWYKTWGQGTLVTVSS

SEQ ID NO: 29 - protein, 354 aa, Artificial Sequence
  AS19584VH28 HCAb
EVQLVESGGGLVQPGGSLRLSCAASGYKYGVYSMGWFRQAPGKGLEGVSAICSGGRTTYSDSVKGRFTISRDNSNQILYLQMNSLRAEDTAVYYCAARPLWTGDCDLSSSWYKTWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 351 aa, Artificial Sequence
  VHH2 HCAb
EVQLVESGGGLVQPGGSLRLSCAASGYTVSSYCMGWFRQAPGKGREGVSAIDSDGSVSYADSVKGRFTISKDNSKNTLYLQMNSLRAEDTAVYFCAADLCWVDQDQGEYNTWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 125 aa, Artificial Sequence
  AS19584VH28 sdAb
EVQLVESGGGLVQPGGSLRLSCAASGYKYGVYSMGWFRQAPGKGLEGVSAICSGGRTTYSDSVKGRFTISRDNSNQILYLQMNSLRAEDTAVYYCAARPLWTGDCDLSSSWYKTWGQGTLVTVSS

SEQ ID NO: 32 - protein, 122 aa, Artificial Sequence
  VHH2 sdAb
EVQLVESGGGLVQPGGSLRLSCAASGYTVSSYCMGWFRQAPGKGREGVSAIDSDGSVSYADSVKGRFTISKDNSKNTLYLQMNSLRAEDTAVYFCAADLCWVDQDQGEYNTWGQGTLVTVSS